SEQUENCE LISTING (309 sequences)

SEQ ID NO: 1 - protein, 3 aa, Artificial Sequence
  Description of Artificial Sequence Synthetic peptide
SNK

SEQ ID NO: 2 - protein, 5 aa, Artificial Sequence
  Description of Artificial Sequence Synthetic peptide
GSNKG

SEQ ID NO: 3 - protein, 7 aa, Artificial Sequence
  Description of Artificial Sequence Synthetic peptide
CGSNKGG

SEQ ID NO: 4 - protein, 4 aa, Artificial Sequence
  Description of Artificial Sequence Synthetic peptide
GSNK

SEQ ID NO: 5 - protein, 4 aa, Artificial Sequence
  Description of Artificial Sequence Synthetic peptide
SNKG

SEQ ID NO: 6 - protein, 5 aa, Artificial Sequence
  Description of Artificial Sequence Synthetic peptide
CSNKG

SEQ ID NO: 7 - protein, 7 aa, Artificial Sequence
  Description of Artificial Sequence Synthetic peptide
CGSNKGC

SEQ ID NO: 8 - protein, 8 aa, Artificial Sequence
  Description of Artificial Sequence Synthetic peptide
CCGSNKGC

SEQ ID NO: 9 - protein, 7 aa, Artificial Sequence
  Description of Artificial Sequence Synthetic peptide
GGSNKGC

SEQ ID NO: 10 - DNA, 321 bp, Artificial Sequence
  Description of Artificial Sequence Synthetic polynucleotide
gacatccagatgacccagtctccatcctccttatctgcctctctgggagaaagagtcagtctcacttgtcgggcaagtcaggaaattagtggttacttaacctggcttcagcagaaaccagatggaactattaaacgcctgatctacgccgcatccactttagattctggtgtcccaaaaaggttcagtggcagtaggtctgggtcagattattctctcaccatcagcagccttgagtctgaagattttgcagactataactgtctacaatatgctaattatcctcggacgttcggtggaggcaccaagctggaaatcaaa

SEQ ID NO: 11 - protein, 107 aa, Artificial Sequence
  Description of Artificial Sequence Synthetic polypeptide
DIQMTQSPSSLSASLGERVSLTCRASQEISGYLTWLQQKPDGTIKRLIYAASTLDSGVPKRFSGSRSGSDYSLTISSLESEDFADYNCLQYANYPRTFGGGTKLEIK

SEQ ID NO: 12 - protein, 6 aa, Artificial Sequence
  Description of Artificial Sequence Synthetic peptide
QEISGY

SEQ ID NO: 13 - protein, 8 aa, Artificial Sequence
  Description of Artificial Sequence Synthetic peptide
AASTLDSG

SEQ ID NO: 14 - protein, 9 aa, Artificial Sequence
  Description of Artificial Sequence Synthetic peptide
LQYANYPRT

SEQ ID NO: 15 - DNA, 348 bp, Artificial Sequence
  Description of Artificial Sequence Synthetic polynucleotide
caggtccagctgcagcagtctggacctgagctggtgaagcctggggcttcagtgaggatatcctgcaaggcttctggctacatattcacaagctactatatacagtgggtgatacacaggcctggacagggacttgagtggattggatggatttatcctggaaatgttaatactaagtacaatgagaagttcaagggcaaggccacactgactgcagacaaatcctccagcacagcctacatgcagctcagcagattgacctctgaggactctgcggtctatttctgtgcaaggatggattacgaggctcactactggggccaaggcaccactctcacagtctcctca

SEQ ID NO: 16 - protein, 116 aa, Artificial Sequence
  Description of Artificial Sequence Synthetic polypeptide
QVQLQQSGPELVKPGASVRISCKASGYIFTSYYIQWVIHRPGQGLEWIGWIYPGNVNTKYNEKFKGKATLTADKSSSTAYMQLSRLTSEDSAVYFCARMDYEAHYWGQGTTLTVSS

SEQ ID NO: 17 - protein, 8 aa, Artificial Sequence
  Description of Artificial Sequence Synthetic peptide
GYIFTSYY

SEQ ID NO: 18 - protein, 8 aa, Artificial Sequence
  Description of Artificial Sequence Synthetic peptide
IYPGNVNT

SEQ ID NO: 19 - protein, 9 aa, Artificial Sequence
  Description of Artificial Sequence Synthetic peptide
ARMDYEAHY

SEQ ID NO: 20 - DNA, 24 bp, Artificial Sequence
  Description of Artificial Sequence Synthetic oligonucleotide
ggatatacattcacctcttactat

SEQ ID NO: 21 - protein, 8 aa, Artificial Sequence
  Description of Artificial Sequence Synthetic peptide
GYTFTSYY

SEQ ID NO: 22 - protein, 25 aa, Artificial Sequence
  Description of Artificial Sequence Synthetic peptide
QVQLQQSGPEVKKPGASVKISCKAS

SEQ ID NO: 23 - protein, 25 aa, Artificial Sequence [sequence omitted]

SEQ ID NO: 24 - protein, 25 aa, Artificial Sequence
  Description of Artificial Sequence Synthetic peptide
QVQLQQSGPEVVKPGASVKISCKAS

SEQ ID NO: 25 - DNA, 51 bp, Artificial Sequence
  Description of Artificial Sequence Synthetic oligonucleotide
atccagtgggtcatccacgcacctggtcagggactggaatggatcggatgg

SEQ ID NO: 26 - protein, 17 aa, Artificial Sequence
  Description of Artificial Sequence Synthetic peptide
IQWVIHAPGQGLEWIGW

SEQ ID NO: 27 - protein, 17 aa, Artificial Sequence
  Description of Artificial Sequence Synthetic peptide
IQWVRQAPGQRLEWMGW

SEQ ID NO: 28 - DNA, 51 bp, Artificial Sequence
  Description of Artificial Sequence Synthetic oligonucleotide
atccagtgggtcatccacaggcctggtcagggactggaatggatcggatgg

SEQ ID NO: 29 - protein, 17 aa, Artificial Sequence
  Description of Artificial Sequence Synthetic peptide
IQWVIHRPGQGLEWIGW

SEQ ID NO: 30 - DNA, 51 bp, Artificial Sequence
  Description of Artificial Sequence Synthetic oligonucleotide
atccagtgggtcaagcacgcacctggtcagggactggaatggatcggatgg

SEQ ID NO: 31 - protein, 17 aa, Artificial Sequence
  Description of Artificial Sequence Synthetic peptide
IQWVKHAPGQGLEWIGW

SEQ ID NO: 32 - DNA, 51 bp, Artificial Sequence
  Description of Artificial Sequence Synthetic oligonucleotide
atccagtgggtcaagcagaggcctggtcagggactggaatggatcggatgg

SEQ ID NO: 33 - protein, 17 aa, Artificial Sequence
  Description of Artificial Sequence Synthetic peptide
IQWVKQRPGQGLEWIGW

SEQ ID NO: 34 - DNA, 51 bp, Artificial Sequence
  Description of Artificial Sequence Synthetic oligonucleotide
atccagtgggtcatccagaggcctggtcagggactggaatggatcggatgg

SEQ ID NO: 35 - protein, 17 aa, Artificial Sequence
  Description of Artificial Sequence Synthetic peptide
IQWVIQRPGQGLEWIGW

SEQ ID NO: 36 - DNA, 51 bp, Artificial Sequence
  Description of Artificial Sequence Synthetic oligonucleotide
atccagtgggtcaagcaggcacctggtcagggactggaatggatcggatgg

SEQ ID NO: 37 - protein, 17 aa, Artificial Sequence
  Description of Artificial Sequence Synthetic peptide
IQWVKQAPGQGLEWIGW

SEQ ID NO: 38 - protein, 17 aa, Artificial Sequence
  Description of Artificial Sequence Synthetic peptide
IQWVKHAPGQGLEWIGW

SEQ ID NO: 39 - protein, 17 aa, Artificial Sequence
  Description of Artificial Sequence Synthetic peptide
IQWVKQRPGQGLEWIGW

SEQ ID NO: 40 - protein, 17 aa, Artificial Sequence
  Description of Artificial Sequence Synthetic peptide
IQWVIQRPGQGLEWIGW

SEQ ID NO: 41 - protein, 17 aa, Artificial Sequence
  Description of Artificial Sequence Synthetic peptide
IQWVKHRPGQGLEWIGW

SEQ ID NO: 42 - protein, 17 aa, Artificial Sequence
  Description of Artificial Sequence Synthetic peptide
IQWVIHAPGQGLEWIAW

SEQ ID NO: 43 - protein, 17 aa, Artificial Sequence
  Description of Artificial Sequence Synthetic peptide
IQWVIHAPGQGLEWVAY

SEQ ID NO: 44 - protein, 17 aa, Artificial Sequence
  Description of Artificial Sequence Synthetic peptide
IQWVIHAPGQGLEWVAW

SEQ ID NO: 45 - protein, 38 aa, Artificial Sequence
  Description of Artificial Sequence Synthetic polypeptide
KYNEKFKGKATLTADKSSSTAYMELSSLTSEDTAVYFC

SEQ ID NO: 46 - protein, 38 aa, Artificial Sequence
  Description of Artificial Sequence Synthetic polypeptide
KYNEKFKGRVTITRDTSASTAYMELSSLRSEDTAVYYC

SEQ ID NO: 47 - protein, 38 aa, Artificial Sequence
  Description of Artificial Sequence Synthetic polypeptide
KYNEKFKGKATLTADKSSSTAYMELSSLRSEDSAVYFC

SEQ ID NO: 48 - protein, 11 aa, Artificial Sequence
  Description of Artificial Sequence Synthetic peptide
WGQGTTVTVSS

SEQ ID NO: 49 - protein, 11 aa, Artificial Sequence
  Description of Artificial Sequence Synthetic peptide
WGQGTLVTVSS

SEQ ID NO: 50 - protein, 26 aa, Artificial Sequence [sequence omitted]

SEQ ID NO: 51 - protein, 26 aa, Artificial Sequence
  Description of Artificial Sequence Synthetic peptide
DIQMTQSPSSLSASLGDRVTLTCRAS

SEQ ID NO: 52 - protein, 17 aa, Artificial Sequence
  Description of Artificial Sequence Synthetic peptide
LTWLQQKPEGAIKRLIY

SEQ ID NO: 53 - protein, 17 aa, Artificial Sequence
  Description of Artificial Sequence Synthetic peptide
LTWYQQKPEKAPKSLIY

SEQ ID NO: 54 - protein, 17 aa, Artificial Sequence
  Description of Artificial Sequence Synthetic peptide
LTWLQQKPEKAIKRLIY

SEQ ID NO: 55 - protein, 31 aa, Artificial Sequence
  Description of Artificial Sequence Synthetic polypeptide
VPKRFSGSRSGSDYSLTISSLQPEDFATYNC

SEQ ID NO: 56 - protein, 31 aa, Artificial Sequence
  Description of Artificial Sequence Synthetic polypeptide
VPSRFSGSGSGTDFTLTISSLQPEDFATYYC

SEQ ID NO: 57 - protein, 31 aa, Artificial Sequence
  Description of Artificial Sequence Synthetic polypeptide
VPKRFSGSRSGSDYTLTISSLQPEDFADYNC

SEQ ID NO: 58 - protein, 10 aa, Artificial Sequence
  Description of Artificial Sequence Synthetic peptide
FGGGTKLEIK

SEQ ID NO: 59 - protein, 10 aa, Artificial Sequence
  Description of Artificial Sequence Synthetic peptide
FGGGTKLEIK

SEQ ID NO: 60 - DNA, 321 bp, Artificial Sequence
  Description of Artificial Sequence Synthetic polynucleotide
gacattcagatgacccagagccctagttcactgagtgcctcagtcggggatcgagtgactatcacctgtcgtgctagtcaggaaatttcaggttacctgacctggtatcagcagaagccagagaaagcccccaagagcctgatctacgctgcatccaccctggacagcggagtgccatctcgattctccggaagcgggtctggtacagactttacactgactatttccagcctgcagccagaggatttcgcaacttactattgcctgcagtacgccaactatcccagaacctttggcggagggacaaaagtggaaatcaag

SEQ ID NO: 61 - protein, 107 aa, Artificial Sequence
  Description of Artificial Sequence Synthetic polypeptide
DIQMTQSPSSLSASVGDRVTITCRASQEISGYLTWYQQKPEKAPKSLIYAASTLDSGVPSRFSGSGSGTDFTLTISSLQPEDFATYYCLQYANYPRTFGGGTKVEIK

SEQ ID NO: 62 - DNA, 348 bp, Artificial Sequence
  Description of Artificial Sequence Synthetic polynucleotide
caggtgcagctggtccagtcaggcgcagaagtgaaaaaacccggagcatcagtcaaagtctcttgtaaggctagcggatatacattcacctcttactatatccagtgggtgagacaggctccaggacagcgcctggaatggatgggctggatctaccccggaaacgtcaatacaaagtataacgagaagttcaaaggaagggtgactatcacccgggacacatcagcatccactgcctacatggagctgtccagcctgagatccgaagacactgccgtgtactattgcgctcgcatggattacgaagcccactattggggtcagggcactctggtcaccgtgtctagt

SEQ ID NO: 63 - protein, 116 aa, Artificial Sequence
  Description of Artificial Sequence Synthetic polypeptide
QVQLVQSGAEVKKPGASVKVSCKASGYTFTSYYIQWVRQAPGQRLEWMGWIYPGNVNTKYNEKFKGRVTITRDTSASTAYMELSSLRSEDTAVYYCARMDYEAHYWGQGTLVTVSS

SEQ ID NO: 64 - DNA, 321 bp, Artificial Sequence
  Description of Artificial Sequence Synthetic polynucleotide
gacattcagatgactcagtctcccagctctctgtcagcctccgtcggcgatagagtgacaatcacttgtcgcgcttcccaggaaattagcggatacctgacttggctgcagcagaaacccgagggggccatcaagcgactgatctacgctgcatctaccctggacagtggagtgcctaagaggttcagcggttctcggagtggctcagactactctctgactatcagttcactgcagcccgaggatttcgcaacctataactgcctgcagtacgccaattatcctagaacatttggcggagggactaaactggaaatcaag

SEQ ID NO: 65 - protein, 107 aa, Artificial Sequence
  Description of Artificial Sequence Synthetic polypeptide
DIQMTQSPSSLSASVGDRVTITCRASQEISGYLTWLQQKPEGAIKRLIYAASTLDSGVPKRFSGSRSGSDYSLTISSLQPEDFATYNCLQYANYPRTFGGGTKLEIK

SEQ ID NO: 66 - DNA, 348 bp, Artificial Sequence
  Description of Artificial Sequence Synthetic polynucleotide
caggtccagctgcagcagagcggtcccgaggtcaaaaaacccggcgcatccgtgaaaatcagttgtaaagcatccggttatatcttcacctcctactatatccagtgggtcatccacgcacctggtcagggactggaatggatcggatggatctaccctgggaacgtgaatacaaagtataacgagaagttcaaaggcaaggctacactgactgcagacaagtccagctctactgcatacatggagctgagttcactgactagcgaagacaccgccgtgtatttctgcgctagaatggattacgaagcccactattggggacaggggaccacagtcaccgtgtcctcc

SEQ ID NO: 67 - protein, 116 aa, Artificial Sequence
  Description of Artificial Sequence Synthetic polypeptide
QVQLQQSGPEVKKPGASVKISCKASGYIFTSYYIQWVIHAPGQGLEWIGWIYPGNVNTKYNEKFKGKATLTADKSSSTAYMELSSLTSEDTAVYFCARMDYEAHYWGQGTTVTVSS

SEQ ID NO: 68 - DNA, 321 bp, Artificial Sequence
  Description of Artificial Sequence Synthetic polynucleotide
gacattcagatgacacagagcccaagctctctgtcagcctccctgggcgacagagtgactctgacctgtcgcgcttctcaggaaatcagtggctacctgacatggctgcagcagaaacccgagaaggccatcaaaagactgatctacgctgcatcaactctggactccggcgtgcctaagaggttcagcggttctcggagtggctcagattacacactgactattagttcactgcagcccgaggacttcgcagattataactgcctgcagtacgccaattatcctcgaacatttggcggagggactaagctggaaatcaaa

SEQ ID NO: 69 - protein, 107 aa, Artificial Sequence
  Description of Artificial Sequence Synthetic polypeptide
DIQMTQSPSSLSASLGDRVTLTCRASQEISGYLTWLQQKPEKAIKRLIYAASTLDSGVPKRFSGSRSGSDYTLTISSLQPEDFADYNCLQYANYPRTFGGGTKLEIK

SEQ ID NO: 70 - DNA, 348 bp, Artificial Sequence
  Description of Artificial Sequence Synthetic polynucleotide
caggtccagctgcagcagagcggtcccgaggtcgtgaaacccggagcatctgtgaaaatcagttgtaaggccagcggatacatctttacctcttactatatccagtgggtcatccacgcacctggtcagggactggaatggatcggatggatctaccctgggaacgtgaataccaagtataacgagaagttcaaaggcaaggctactctgaccgcagacaagtccagctctacagcatacatggagctgagttcactgaggtccgaagacagcgccgtgtatttctgcgctcggatggattacgaagcccactattggggacaggggactctggtcaccgtgtcctcc

SEQ ID NO: 71 - protein, 116 aa, Artificial Sequence
  Description of Artificial Sequence Synthetic polypeptide
QVQLQQSGPEVVKPGASVKISCKASGYIFTSYYIQWVIHAPGQGLEWIGWIYPGNVNTKYNEKFKGKATLTADKSSSTAYMELSSLRSEDSAVYFCARMDYEAHYWGQGTLVTVSS

SEQ ID NO: 72 - DNA, 322 bp, Artificial Sequence
  Description of Artificial Sequence Synthetic polynucleotide
gacatccagatgacccagtctccatcctccttatctgcctctctgggagaaagagtcagtctcacttgtcgggcaagtcaggaaattagtggttacttaacctggcttcagcagaaaccagatggaactattaaacgcctgatctacgccgcatccactttagattctggtgtcccaaaaaggttcagtggcagtaggtctgggtcagattattctctcaccatcagcagccttgagtctgaagattttgcagactataactgtctacaatatgctaattatcctcggacgttcggtggaggcaccaagctggaaatcaaac

SEQ ID NO: 73 - DNA, 349 bp, Artificial Sequence
  Description of Artificial Sequence Synthetic polynucleotide
caggtccagctgcagcagtctggacctgagctggtgaagcctggggcttcagtgaggatatcctgcaaggcttctggctacatattcacaagctactatatacagtgggtgatacacaggcctggacagggacttgagtggattggatggatttatcctggaaatgttaatactaagtacaatgagaagttcaagggcaaggccacactgactgcagacaaatcctccagcacagcctacatgcagctcagcagattgacctctgaggactctgcggtctatttctgtgcaaggatggattacgaggctcactactggggccaaggcaccactctcacagtctcctcag

SEQ ID NO: 74 - DNA, 322 bp, Artificial Sequence
  Description of Artificial Sequence Synthetic polynucleotide
gacattcagatgacccagagccctagttcactgagtgcctcagtcggggatcgagtgactatcacctgtcgtgctagtcaggaaatttcaggttacctgacctggtatcagcagaagccagagaaagcccccaagagcctgatctacgctgcatccaccctggacagcggagtgccatctcgattctccggaagcgggtctggtacagactttacactgactatttccagcctgcagccagaggatttcgcaacttactattgcctgcagtacgccaactatcccagaacctttggcggagggacaaaagtggaaatcaagc

SEQ ID NO: 75 - DNA, 349 bp, Artificial Sequence
  Description of Artificial Sequence Synthetic polynucleotide
caggtgcagctggtccagtcaggcgcagaagtgaaaaaacccggagcatcagtcaaagtctcttgtaaggctagcggatatacattcacctcttactatatccagtgggtgagacaggctccaggacagcgcctggaatggatgggctggatctaccccggaaacgtcaatacaaagtataacgagaagttcaaaggaagggtgactatcacccgggacacatcagcatccactgcctacatggagctgtccagcctgagatccgaagacactgccgtgtactattgcgctcgcatggattacgaagcccactattggggtcagggcactctggtcaccgtgtctagtg

SEQ ID NO: 76 - DNA, 322 bp, Artificial Sequence
  Description of Artificial Sequence Synthetic polynucleotide
gacattcagatgactcagtctcccagctctctgtcagcctccgtcggcgatagagtgacaatcacttgtcgcgcttcccaggaaattagcggatacctgacttggctgcagcagaaacccgagggggccatcaagcgactgatctacgctgcatctaccctggacagtggagtgcctaagaggttcagcggttctcggagtggctcagactactctctgactatcagttcactgcagcccgaggatttcgcaacctataactgcctgcagtacgccaattatcctagaacatttggcggagggactaaactggaaatcaagc

SEQ ID NO: 77 - DNA, 349 bp, Artificial Sequence
  Description of Artificial Sequence Synthetic polynucleotide
caggtccagctgcagcagagcggtcccgaggtcaaaaaacccggcgcatccgtgaaaatcagttgtaaagcatccggttatatcttcacctcctactatatccagtgggtcatccacgcacctggtcagggactggaatggatcggatggatctaccctgggaacgtgaatacaaagtataacgagaagttcaaaggcaaggctacactgactgcagacaagtccagctctactgcatacatggagctgagttcactgactagcgaagacaccgccgtgtatttctgcgctagaatggattacgaagcccactattggggacaggggaccacagtcaccgtgtcctccg

SEQ ID NO: 78 - DNA, 322 bp, Artificial Sequence
  Description of Artificial Sequence Synthetic polynucleotide
gacattcagatgacacagagcccaagctctctgtcagcctccctgggcgacagagtgactctgacctgtcgcgcttctcaggaaatcagtggctacctgacatggctgcagcagaaacccgagaaggccatcaaaagactgatctacgctgcatcaactctggactccggcgtgcctaagaggttcagcggttctcggagtggctcagattacacactgactattagttcactgcagcccgaggacttcgcagattataactgcctgcagtacgccaattatcctcgaacatttggcggagggactaagctggaaatcaaac

SEQ ID NO: 79 - DNA, 349 bp, Artificial Sequence
  Description of Artificial Sequence Synthetic polynucleotide
caggtccagctgcagcagagcggtcccgaggtcgtgaaacccggagcatctgtgaaaatcagttgtaaggccagcggatacatctttacctcttactatatccagtgggtcatccacgcacctggtcagggactggaatggatcggatggatctaccctgggaacgtgaataccaagtataacgagaagttcaaaggcaaggctactctgaccgcagacaagtccagctctacagcatacatggagctgagttcactgaggtccgaagacagcgccgtgtatttctgcgctcggatggattacgaagcccactattggggacaggggactctggtcaccgtgtcctccg

SEQ ID NO: 80 - DNA, 322 bp, Artificial Sequence
  Description of Artificial Sequence Synthetic polynucleotide
gatattcagatgacccagagcccatcatccctgtctgccagtctgggcgagagagtgtctctgacctgtcgcgcttcccaggaaatcagcggatacctgacctggctgcagcagaaacccgacgggacaatcaagagactgatctacgctgcatctactctggatagtggagtgcctaagaggttctcaggttcccggagcggctctgactacagtctgaccattagctctctggagtccgaagacttcgcagattataactgcctgcagtacgccaattatccaagaacctttggcggagggacaaaactggaaatcaagc

SEQ ID NO: 81 - DNA, 27 bp, Artificial Sequence
  Description of Artificial Sequence Synthetic oligonucleotide
gtgcagtacgccaattatcctagaaca

SEQ ID NO: 82 - protein, 9 aa, Artificial Sequence
  Description of Artificial Sequence Synthetic peptide
VQYANYPRT

SEQ ID NO: 83 - DNA, 27 bp, Artificial Sequence
  Description of Artificial Sequence Synthetic oligonucleotide
atccagtacgccaattatcctagaaca

SEQ ID NO: 84 - protein, 9 aa, Artificial Sequence
  Description of Artificial Sequence Synthetic peptide
IQYANYPRT

SEQ ID NO: 85 - DNA, 27 bp, Artificial Sequence
  Description of Artificial Sequence Synthetic oligonucleotide
ctgaactacgccaattatcctagaaca

SEQ ID NO: 86 - protein, 9 aa, Artificial Sequence
  Description of Artificial Sequence Synthetic peptide
LNYANYPRT

SEQ ID NO: 87 - DNA, 27 bp, Artificial Sequence
  Description of Artificial Sequence Synthetic oligonucleotide
ctggagtacgccaattatcctagaaca

SEQ ID NO: 88 - protein, 9 aa, Artificial Sequence
  Description of Artificial Sequence Synthetic peptide
LEYANYPRT

SEQ ID NO: 89 - DNA, 27 bp, Artificial Sequence
  Description of Artificial Sequence Synthetic oligonucleotide
ctgcagttcgccaattatcctagaaca

SEQ ID NO: 90 - protein, 9 aa, Artificial Sequence
  Description of Artificial Sequence Synthetic peptide
LQFANYPRT

SEQ ID NO: 91 - DNA, 27 bp, Artificial Sequence
  Description of Artificial Sequence Synthetic oligonucleotide
ctgcagtacgcccagtatcctagaaca

SEQ ID NO: 92 - protein, 9 aa, Artificial Sequence
  Description of Artificial Sequence Synthetic peptide
LQYAQYPRT

SEQ ID NO: 93 - DNA, 27 bp, Artificial Sequence
  Description of Artificial Sequence Synthetic oligonucleotide
ctgcagtacgccgactatcctagaaca

SEQ ID NO: 94 - protein, 9 aa, Artificial Sequence
  Description of Artificial Sequence Synthetic peptide
LQYADYPRT

SEQ ID NO: 95 - DNA, 27 bp, Artificial Sequence
  Description of Artificial Sequence Synthetic oligonucleotide
ctgcagtacgccaatttccctagaaca

SEQ ID NO: 96 - protein, 9 aa, Artificial Sequence
  Description of Artificial Sequence Synthetic peptide
LQYANFPRT

SEQ ID NO: 97 - DNA, 27 bp, Artificial Sequence
  Description of Artificial Sequence Synthetic oligonucleotide
gctagaaaggattacgaagcccactat

SEQ ID NO: 98 - protein, 9 aa, Artificial Sequence
  Description of Artificial Sequence Synthetic peptide
ARKDYEAHY

SEQ ID NO: 99 - DNA, 27 bp, Artificial Sequence
  Description of Artificial Sequence Synthetic oligonucleotide
gctagaatcgattacgaagcccactat

SEQ ID NO: 100 - protein, 9 aa, Artificial Sequence
  Description of Artificial Sequence Synthetic peptide
ARIDYEAHY

SEQ ID NO: 101 - DNA, 27 bp, Artificial Sequence
  Description of Artificial Sequence Synthetic oligonucleotide
gctagaatggagtacgaagcccactat

SEQ ID NO: 102 - protein, 9 aa, Artificial Sequence
  Description of Artificial Sequence Synthetic peptide
ARMEYEAHY

SEQ ID NO: 103 - DNA, 27 bp, Artificial Sequence
  Description of Artificial Sequence Synthetic oligonucleotide
gctagaatgtcctacgaagcccactat

SEQ ID NO: 104 - protein, 9 aa, Artificial Sequence
  Description of Artificial Sequence Synthetic peptide
ARMSYEAHY

SEQ ID NO: 105 - DNA, 27 bp, Artificial Sequence
  Description of Artificial Sequence Synthetic oligonucleotide
gctagaatgaactacgaagcccactat

SEQ ID NO: 106 - protein, 9 aa, Artificial Sequence
  Description of Artificial Sequence Synthetic peptide
ARMNYEAHY

SEQ ID NO: 107 - DNA, 27 bp, Artificial Sequence
  Description of Artificial Sequence Synthetic oligonucleotide
gctagaatggatttcgaagcccactat

SEQ ID NO: 108 - protein, 9 aa, Artificial Sequence
  Description of Artificial Sequence Synthetic peptide
ARMDFEAHY

SEQ ID NO: 109 - DNA, 27 bp, Artificial Sequence
  Description of Artificial Sequence Synthetic oligonucleotide
gctagaatggattaccaggcccactat

SEQ ID NO: 110 - protein, 9 aa, Artificial Sequence
  Description of Artificial Sequence Synthetic peptide
ARMDYQAHY

SEQ ID NO: 111 - DNA, 27 bp, Artificial Sequence
  Description of Artificial Sequence Synthetic oligonucleotide
gctagaatggattacgacgcccactat

SEQ ID NO: 112 - protein, 9 aa, Artificial Sequence
  Description of Artificial Sequence Synthetic peptide
ARMDYDAHY

SEQ ID NO: 113 - DNA, 27 bp, Artificial Sequence
  Description of Artificial Sequence Synthetic oligonucleotide
gctagaatggattacgaaggccactat

SEQ ID NO: 114 - protein, 9 aa, Artificial Sequence
  Description of Artificial Sequence Synthetic peptide
ARMDYEGHY

SEQ ID NO: 115 - DNA, 27 bp, Artificial Sequence
  Description of Artificial Sequence Synthetic oligonucleotide
gctagaatggattacgaagtccactat

SEQ ID NO: 116 - protein, 9 aa, Artificial Sequence
  Description of Artificial Sequence Synthetic peptide
ARMDYEVHY

SEQ ID NO: 117 - DNA, 27 bp, Artificial Sequence
  Description of Artificial Sequence Synthetic oligonucleotide
gctagaatggattacgaagccttctat

SEQ ID NO: 118 - protein, 9 aa, Artificial Sequence
  Description of Artificial Sequence Synthetic peptide
ARMDYEAFY

SEQ ID NO: 119 - DNA, 27 bp, Artificial Sequence
  Description of Artificial Sequence Synthetic oligonucleotide
gctagaatggattacgaagccaactat

SEQ ID NO: 120 - protein, 9 aa, Artificial Sequence
  Description of Artificial Sequence Synthetic peptide
ARMDYEANY

SEQ ID NO: 121 - DNA, 27 bp, Artificial Sequence
  Description of Artificial Sequence Synthetic oligonucleotide
gctagaatggattacgaagcccacttt

SEQ ID NO: 122 - protein, 9 aa, Artificial Sequence
  Description of Artificial Sequence Synthetic peptide
ARMDYEAHF

SEQ ID NO: 123 - DNA, 349 bp, Artificial Sequence
  Description of Artificial Sequence Synthetic polynucleotide
caggtccagctgcagcagagcggtcccgaggtcaaaaaacccggcgcatccgtgaaaatcagttgtaaagcatccggttatatcttcacctcctactatatccagtgggtcatccacaggcctggtcagggactggaatggatcggatggatctaccctgggaacgtgaatacaaagtataacgagaagttcaaaggcaaggctacactgactgcagacaagtccagctctactgcatacatggagctgagttcactgactagcgaagacaccgccgtgtatttctgcgctagaatggattacgaagcccactattggggacaggggaccacagtcaccgtgtcctccg

SEQ ID NO: 124 - protein, 116 aa, Artificial Sequence
  Description of Artificial Sequence Synthetic polypeptide
QVQLQQSGPEVKKPGASVKISCKASGYIFTSYYIQWVIHRPGQGLEWIGWIYPGNVNTKYNEKFKGKATLTADKSSSTAYMELSSLTSEDTAVYFCARMDYEAHYWGQGTTVTVSS

SEQ ID NO: 125 - DNA, 349 bp, Artificial Sequence
  Description of Artificial Sequence Synthetic polynucleotide
caggtccagctgcagcagagcggtcccgaggtcaaaaaacccggcgcatccgtgaaaatcagttgtaaagcatccggttatatcttcacctcctactatatccagtgggtcaagcacgcacctggtcagggactggaatggatcggatggatctaccctgggaacgtgaatacaaagtataacgagaagttcaaaggcaaggctacactgactgcagacaagtccagctctactgcatacatggagctgagttcactgactagcgaagacaccgccgtgtatttctgcgctagaatggattacgaagcccactattggggacaggggaccacagtcaccgtgtcctccg

SEQ ID NO: 126 - protein, 116 aa, Artificial Sequence
  Description of Artificial Sequence Synthetic polypeptide
QVQLQQSGPEVKKPGASVKISCKASGYIFTSYYIQWVKHAPGQGLEWIGWIYPGNVNTKYNEKFKGKATLTADKSSSTAYMELSSLTSEDTAVYFCARMDYEAHYWGQGTTVTVSS

SEQ ID NO: 127 - DNA, 349 bp, Artificial Sequence
  Description of Artificial Sequence Synthetic polynucleotide
caggtccagctgcagcagagcggtcccgaggtcaaaaaacccggcgcatccgtgaaaatcagttgtaaagcatccggttatatcttcacctcctactatatccagtgggtcaagcagaggcctggtcagggactggaatggatcggatggatctaccctgggaacgtgaatacaaagtataacgagaagttcaaaggcaaggctacactgactgcagacaagtccagctctactgcatacatggagctgagttcactgactagcgaagacaccgccgtgtatttctgcgctagaatggattacgaagcccactattggggacaggggaccacagtcaccgtgtcctccg

SEQ ID NO: 128 - protein, 116 aa, Artificial Sequence
  Description of Artificial Sequence Synthetic polypeptide
QVQLQQSGPEVKKPGASVKISCKASGYIFTSYYIQWVKQRPGQGLEWIGWIYPGNVNTKYNEKFKGKATLTADKSSSTAYMELSSLTSEDTAVYFCARMDYEAHYWGQGTTVTVSS

SEQ ID NO: 129 - DNA, 349 bp, Artificial Sequence
  Description of Artificial Sequence Synthetic polynucleotide
caggtccagctgcagcagagcggtcccgaggtcaaaaaacccggcgcatccgtgaaaatcagttgtaaagcatccggttatatcttcacctcctactatatccagtgggtcatccagaggcctggtcagggactggaatggatcggatggatctaccctgggaacgtgaatacaaagtataacgagaagttcaaaggcaaggctacactgactgcagacaagtccagctctactgcatacatggagctgagttcactgactagcgaagacaccgccgtgtatttctgcgctagaatggattacgaagcccactattggggacaggggaccacagtcaccgtgtcctccg

SEQ ID NO: 130 - protein, 116 aa, Artificial Sequence
  Description of Artificial Sequence Synthetic polypeptide
QVQLQQSGPEVKKPGASVKISCKASGYIFTSYYIQWVIQRPGQGLEWIGWIYPGNVNTKYNEKFKGKATLTADKSSSTAYMELSSLTSEDTAVYFCARMDYEAHYWGQGTTVTVSS

SEQ ID NO: 131 - DNA, 349 bp, Artificial Sequence
  Description of Artificial Sequence Synthetic polynucleotide
caggtccagctgcagcagagcggtcccgaggtcaaaaaacccggcgcatccgtgaaaatcagttgtaaagcatccggttatatcttcacctcctactatatccagtgggtcaagcaggcacctggtcagggactggaatggatcggatggatctaccctgggaacgtgaatacaaagtataacgagaagttcaaaggcaaggctacactgactgcagacaagtccagctctactgcatacatggagctgagttcactgactagcgaagacaccgccgtgtatttctgcgctagaatggattacgaagcccactattggggacaggggaccacagtcaccgtgtcctccg

SEQ ID NO: 132 - protein, 116 aa, Artificial Sequence
  Description of Artificial Sequence Synthetic polypeptide
QVQLQQSGPEVKKPGASVKISCKASGYIFTSYYIQWVKQAPGQGLEWIGWIYPGNVNTKYNEKFKGKATLTADKSSSTAYMELSSLTSEDTAVYFCARMDYEAHYWGQGTTVTVSS

SEQ ID NO: 133 - DNA, 348 bp, Artificial Sequence
  Description of Artificial Sequence Synthetic polynucleotide
caggtccagctgcagcagtctggacctgagctggtgaagcctggggcttcagtgaggatatcctgcaaggcttctggctacatattcacaagctactatatacagtgggtgatccaccgccctggacagggacttgagtggattggatggatttatcctggaaatgttaatactaagtacaatgagaagttcaagggcaaggccacactgactgcagacaaatcctccagcacagcctacatgcagctcagcagattgacctctgaggactctgcggtctatttctgtgcaaggatggattacgaggctcactactggggccaaggcaccactctcacagtctcctca

SEQ ID NO: 134 - protein, 116 aa, Artificial Sequence; identical to SEQ ID NO: 16
  Description of Artificial Sequence Synthetic polypeptide

SEQ ID NO: 135 - DNA, 348 bp, Artificial Sequence
  Description of Artificial Sequence Synthetic polynucleotide
caggtccagctgcagcagtctggacctgagctggtgaagcctggggcttcagtgaggatatcctgcaaggcttctggctacatattcacaagctactatatacagtgggtgaagcaggcccctggacagggacttgagtggattggatggatttatcctggaaatgttaatactaagtacaatgagaagttcaagggcaaggccacactgactgcagacaaatcctccagcacagcctacatgcagctcagcagattgacctctgaggactctgcggtctatttctgtgcaaggatggattacgaggctcactactggggccaaggcaccactctcacagtctcctca

SEQ ID NO: 136 - protein, 116 aa, Artificial Sequence
  Description of Artificial Sequence Synthetic polypeptide
QVQLQQSGPELVKPGASVRISCKASGYIFTSYYIQWVKQAPGQGLEWIGWIYPGNVNTKYNEKFKGKATLTADKSSSTAYMQLSRLTSEDSAVYFCARMDYEAHYWGQGTTLTVSS

SEQ ID NO: 137 - DNA, 348 bp, Artificial Sequence
  Description of Artificial Sequence Synthetic polynucleotide
caggtccagctgcagcagtctggacctgagctggtgaagcctggggcttcagtgaggatatcctgcaaggcttctggctacatattcacaagctactatatacagtgggtgaagcacgcccctggacagggacttgagtggattggatggatttatcctggaaatgttaatactaagtacaatgagaagttcaagggcaaggccacactgactgcagacaaatcctccagcacagcctacatgcagctcagcagattgacctctgaggactctgcggtctatttctgtgcaaggatggattacgaggctcactactggggccaaggcaccactctcacagtctcctca

SEQ ID NO: 138 - protein, 116 aa, Artificial Sequence
  Description of Artificial Sequence Synthetic polypeptide
QVQLQQSGPELVKPGASVRISCKASGYIFTSYYIQWVKHAPGQGLEWIGWIYPGNVNTKYNEKFKGKATLTADKSSSTAYMQLSRLTSEDSAVYFCARMDYEAHYWGQGTTLTVSS

SEQ ID NO: 139 - DNA, 349 bp, Artificial Sequence
  Description of Artificial Sequence Synthetic polynucleotide
caggtccagctgcagcagtctggacctgagctggtgaagcctggggcttcagtgaggatatcctgcaaggcttctggctacatattcacaagctactatatacagtgggtgaagcagaggcctggacagggacttgagtggattggatggatttatcctggaaatgttaatactaagtacaatgagaagttcaagggcaaggccacactgactgcagacaaatcctccagcacagcctacatgcagctcagcagattgacctctgaggactctgcggtctatttctgtgcaaggatggattacgaggctcactactggggccaaggcaccactctcacagtctcctcag

SEQ ID NO: 140 - protein, 116 aa, Artificial Sequence
  Description of Artificial Sequence Synthetic polypeptide
QVQLQQSGPELVKPGASVRISCKASGYIFTSYYIQWVKQRPGQGLEWIGWIYPGNVNTKYNEKFKGKATLTADKSSSTAYMQLSRLTSEDSAVYFCARMDYEAHYWGQGTTLTVSS

SEQ ID NO: 141 - DNA, 348 bp, Artificial Sequence
  Description of Artificial Sequence Synthetic polynucleotide
caggtccagctgcagcagtctggacctgagctggtgaagcctggggcttcagtgaggatatcctgcaaggcttctggctacatattcacaagctactatatacagtgggtgatccagcgccctggacagggacttgagtggattggatggatttatcctggaaatgttaatactaagtacaatgagaagttcaagggcaaggccacactgactgcagacaaatcctccagcacagcctacatgcagctcagcagattgacctctgaggactctgcggtctatttctgtgcaaggatggattacgaggctcactactggggccaaggcaccactctcacagtctcctca

SEQ ID NO: 142 - protein, 116 aa, Artificial Sequence
  Description of Artificial Sequence Synthetic polypeptide
QVQLQQSGPELVKPGASVRISCKASGYIFTSYYIQWVIQRPGQGLEWIGWIYPGNVNTKYNEKFKGKATLTADKSSSTAYMQLSRLTSEDSAVYFCARMDYEAHYWGQGTTLTVSS

SEQ ID NO: 143 - DNA, 348 bp, Artificial Sequence
  Description of Artificial Sequence Synthetic polynucleotide
caggtgcagctggtccagtcaggcgcagaagtgaaaaaacccggagcatcagtcaaagtctcttgtaaggctagcggatatacattcacctcttactatatccagtgggtgatccaccgcccaggacagcgcctggaatggatgggctggatctaccccggaaacgtcaatacaaagtataacgagaagttcaaaggaagggtgactatcacccgggacacatcagcatccactgcctacatggagctgtccagcctgagatccgaagacactgccgtgtactattgcgctcgcatggattacgaagcccactattggggtcagggcactctggtcaccgtgtctagt

SEQ ID NO: 144 - protein, 116 aa, Artificial Sequence
  Description of Artificial Sequence Synthetic polypeptide
QVQLVQSGAEVKKPGASVKVSCKASGYTFTSYYIQWVIHRPGQRLEWMGWIYPGNVNTKYNEKFKGRVTITRDTSASTAYMELSSLRSEDTAVYYCARMDYEAHYWGQGTLVTVSS

SEQ ID NO: 145 - DNA, 348 bp, Artificial Sequence
  Description of Artificial Sequence Synthetic polynucleotide
caggtgcagctggtccagtcaggcgcagaagtgaaaaaacccggagcatcagtcaaagtctcttgtaaggctagcggatatacattcacctcttactatatccagtgggtgaagcaggccccaggacagcgcctggaatggatgggctggatctaccccggaaacgtcaatacaaagtataacgagaagttcaaaggaagggtgactatcacccgggacacatcagcatccactgcctacatggagctgtccagcctgagatccgaagacactgccgtgtactattgcgctcgcatggattacgaagcccactattggggtcagggcactctggtcaccgtgtctagt

SEQ ID NO: 146 - protein, 116 aa, Artificial Sequence
  Description of Artificial Sequence Synthetic polypeptide
QVQLVQSGAEVKKPGASVKVSCKASGYTFTSYYIQWVKQAPGQRLEWMGWIYPGNVNTKYNEKFKGRVTITRDTSASTAYMELSSLRSEDTAVYYCARMDYEAHYWGQGTLVTVSS

SEQ ID NO: 147 - DNA, 348 bp, Artificial Sequence
  Description of Artificial Sequence Synthetic polynucleotide
caggtgcagctggtccagtcaggcgcagaagtgaaaaaacccggagcatcagtcaaagtctcttgtaaggctagcggatatacattcacctcttactatatccagtgggtgaagcacgccccaggacagcgcctggaatggatgggctggatctaccccggaaacgtcaatacaaagtataacgagaagttcaaaggaagggtgactatcacccgggacacatcagcatccactgcctacatggagctgtccagcctgagatccgaagacactgccgtgtactattgcgctcgcatggattacgaagcccactattggggtcagggcactctggtcaccgtgtctagt

SEQ ID NO: 148 - protein, 116 aa, Artificial Sequence
  Description of Artificial Sequence Synthetic polypeptide
QVQLVQSGAEVKKPGASVKVSCKASGYTFTSYYIQWVKHAPGQRLEWMGWIYPGNVNTKYNEKFKGRVTITRDTSASTAYMELSSLRSEDTAVYYCARMDYEAHYWGQGTLVTVSS

SEQ ID NO: 149 - DNA, 348 bp, Artificial Sequence
  Description of Artificial Sequence Synthetic polynucleotide
caggtgcagctggtccagtcaggcgcagaagtgaaaaaacccggagcatcagtcaaagtctcttgtaaggctagcggatatacattcacctcttactatatccagtgggtgaagcagcgcccaggacagcgcctggaatggatgggctggatctaccccggaaacgtcaatacaaagtataacgagaagttcaaaggaagggtgactatcacccgggacacatcagcatccactgcctacatggagctgtccagcctgagatccgaagacactgccgtgtactattgcgctcgcatggattacgaagcccactattggggtcagggcactctggtcaccgtgtctagt

SEQ ID NO: 150 - protein, 116 aa, Artificial Sequence
  Description of Artificial Sequence Synthetic polypeptide
QVQLVQSGAEVKKPGASVKVSCKASGYTFTSYYIQWVKQRPGQRLEWMGWIYPGNVNTKYNEKFKGRVTITRDTSASTAYMELSSLRSEDTAVYYCARMDYEAHYWGQGTLVTVSS

SEQ ID NO: 151 - DNA, 348 bp, Artificial Sequence
  Description of Artificial Sequence Synthetic polynucleotide
caggtgcagctggtccagtcaggcgcagaagtgaaaaaacccggagcatcagtcaaagtctcttgtaaggctagcggatatacattcacctcttactatatccagtgggtgatccagcgcccaggacagcgcctggaatggatgggctggatctaccccggaaacgtcaatacaaagtataacgagaagttcaaaggaagggtgactatcacccgggacacatcagcatccactgcctacatggagctgtccagcctgagatccgaagacactgccgtgtactattgcgctcgcatggattacgaagcccactattggggtcagggcactctggtcaccgtgtctagt

SEQ ID NO: 152 - protein, 116 aa, Artificial Sequence
  Description of Artificial Sequence Synthetic polypeptide
QVQLVQSGAEVKKPGASVKVSCKASGYTFTSYYIQWVIQRPGQRLEWMGWIYPGNVNTKYNEKFKGRVTITRDTSASTAYMELSSLRSEDTAVYYCARMDYEAHYWGQGTLVTVSS

SEQ ID NO: 153 - DNA, 348 bp, Artificial Sequence
  Description of Artificial Sequence Synthetic polynucleotide
caggtccagctgcagcagagcggtcccgaggtcgtgaaacccggagcatctgtgaaaatcagttgtaaggccagcggatacatctttacctcttactatatccagtgggtcatccaccgccctggtcagggactggaatggatcggatggatctaccctgggaacgtgaataccaagtataacgagaagttcaaaggcaaggctactctgaccgcagacaagtccagctctacagcatacatggagctgagttcactgaggtccgaagacagcgccgtgtatttctgcgctcggatggattacgaagcccactattggggacaggggactctggtcaccgtgtcctcc

SEQ ID NO: 154 - protein, 116 aa, Artificial Sequence
  Description of Artificial Sequence Synthetic polypeptide
QVQLQQSGPEVVKPGASVKISCKASGYIFTSYYIQWVIHRPGQGLEWIGWIYPGNVNTKYNEKFKGKATLTADKSSSTAYMELSSLRSEDSAVYFCARMDYEAHYWGQGTLVTVSS

SEQ ID NO: 155 - DNA, 348 bp, Artificial Sequence
  Description of Artificial Sequence Synthetic polynucleotide
caggtccagctgcagcagagcggtcccgaggtcgtgaaacccggagcatctgtgaaaatcagttgtaaggccagcggatacatctttacctcttactatatccagtgggtcaagcaggcccctggtcagggactggaatggatcggatggatctaccctgggaacgtgaataccaagtataacgagaagttcaaaggcaaggctactctgaccgcagacaagtccagctctacagcatacatggagctgagttcactgaggtccgaagacagcgccgtgtatttctgcgctcggatggattacgaagcccactattggggacaggggactctggtcaccgtgtcctcc

SEQ ID NO: 156 - protein, 116 aa, Artificial Sequence
  Description of Artificial Sequence Synthetic polypeptide
QVQLQQSGPEVVKPGASVKISCKASGYIFTSYYIQWVKQAPGQGLEWIGWIYPGNVNTKYNEKFKGKATLTADKSSSTAYMELSSLRSEDSAVYFCARMDYEAHYWGQGTLVTVSS

SEQ ID NO: 157 - DNA, 348 bp, Artificial Sequence
  Description of Artificial Sequence Synthetic polynucleotide
caggtccagctgcagcagagcggtcccgaggtcgtgaaacccggagcatctgtgaaaatcagttgtaaggccagcggatacatctttacctcttactatatccagtgggtcaagcacgcccctggtcagggactggaatggatcggatggatctaccctgggaacgtgaataccaagtataacgagaagttcaaaggcaaggctactctgaccgcagacaagtccagctctacagcatacatggagctgagttcactgaggtccgaagacagcgccgtgtatttctgcgctcggatggattacgaagcccactattggggacaggggactctggtcaccgtgtcctcc

SEQ ID NO: 158 - protein, 116 aa, Artificial Sequence
  Description of Artificial Sequence Synthetic polypeptide
QVQLQQSGPEVVKPGASVKISCKASGYIFTSYYIQWVKHAPGQGLEWIGWIYPGNVNTKYNEKFKGKATLTADKSSSTAYMELSSLRSEDSAVYFCARMDYEAHYWGQGTLVTVSS

SEQ ID NO: 159 - DNA, 348 bp, Artificial Sequence
  Description of Artificial Sequence Synthetic polynucleotide
caggtccagctgcagcagagcggtcccgaggtcgtgaaacccggagcatctgtgaaaatcagttgtaaggccagcggatacatctttacctcttactatatccagtgggtcaagcagcgccctggtcagggactggaatggatcggatggatctaccctgggaacgtgaataccaagtataacgagaagttcaaaggcaaggctactctgaccgcagacaagtccagctctacagcatacatggagctgagttcactgaggtccgaagacagcgccgtgtatttctgcgctcggatggattacgaagcccactattggggacaggggactctggtcaccgtgtcctcc

SEQ ID NO: 160 - protein, 116 aa, Artificial Sequence
  Description of Artificial Sequence Synthetic polypeptide
QVQLQQSGPEVVKPGASVKISCKASGYIFTSYYIQWVKQRPGQGLEWIGWIYPGNVNTKYNEKFKGKATLTADKSSSTAYMELSSLRSEDSAVYFCARMDYEAHYWGQGTLVTVSS

SEQ ID NO: 161 - DNA, 348 bp, Artificial Sequence
  Description of Artificial Sequence Synthetic polynucleotide
caggtccagctgcagcagagcggtcccgaggtcgtgaaacccggagcatctgtgaaaatcagttgtaaggccagcggatacatctttacctcttactatatccagtgggtcatccagcgccctggtcagggactggaatggatcggatggatctaccctgggaacgtgaataccaagtataacgagaagttcaaaggcaaggctactctgaccgcagacaagtccagctctacagcatacatggagctgagttcactgaggtccgaagacagcgccgtgtatttctgcgctcggatggattacgaagcccactattggggacaggggactctggtcaccgtgtcctcc

SEQ ID NO: 162 - protein, 116 aa, Artificial Sequence
  Description of Artificial Sequence Synthetic polypeptide
QVQLQQSGPEVVKPGASVKISCKASGYIFTSYYIQWVIQRPGQGLEWIGWIYPGNVNTKYNEKFKGKATLTADKSSSTAYMELSSLRSEDSAVYFCARMDYEAHYWGQGTLVTVSS

SEQ ID NO: 163 - DNA, 33 bp, Artificial Sequence
  Description of Artificial Sequence Synthetic oligonucleotide
gacgcttcccaggaaattagcggatacctgact

SEQ ID NO: 164 - protein, 11 aa, Artificial Sequence
  Description of Artificial Sequence Synthetic peptide
DASQEISGYLT

SEQ ID NO: 165 - DNA, 33 bp, Artificial Sequence
  Description of Artificial Sequence Synthetic oligonucleotide
cgcgcgacccaggaaattagcggatacctgact

SEQ ID NO: 166 - protein, 11 aa, Artificial Sequence
  Description of Artificial Sequence Synthetic peptide
RATQEISGYLT

SEQ ID NO: 167 - DNA, 18 bp, Artificial Sequence
  Description of Artificial Sequence Synthetic oligonucleotide
gacgaaattagcggatac

SEQ ID NO: 168 - protein, 6 aa, Artificial Sequence
  Description of Artificial Sequence Synthetic peptide
DEISGY

SEQ ID NO: 169 - DNA, 18 bp, Artificial Sequence
  Description of Artificial Sequence Synthetic oligonucleotide
caggacattagcggatac

SEQ ID NO: 170 - protein, 6 aa, Artificial Sequence
  Description of Artificial Sequence Synthetic peptide
QDISGY

SEQ ID NO: 171 - DNA, 18 bp, Artificial Sequence
  Description of Artificial Sequence Synthetic oligonucleotide
caggaaattgacggatac

SEQ ID NO: 172 - protein, 6 aa, Artificial Sequence
  Description of Artificial Sequence Synthetic peptide
QEIDGY

SEQ ID NO: 173 - DNA, 24 bp, Artificial Sequence
  Description of Artificial Sequence Synthetic oligonucleotide
gacgcatctaccctggacagtgga

SEQ ID NO: 174 - protein, 8 aa, Artificial Sequence
  Description of Artificial Sequence Synthetic peptide
DASTLDSG

SEQ ID NO: 175 - DNA, 24 bp, Artificial Sequence
  Description of Artificial Sequence Synthetic oligonucleotide
gctgcagacaccctggacagtgga

SEQ ID NO: 176 - protein, 8 aa, Artificial Sequence
  Description of Artificial Sequence Synthetic peptide
AADTLDSG

SEQ ID NO: 177 - DNA, 24 bp, Artificial Sequence
  Description of Artificial Sequence Synthetic oligonucleotide
gctgcatctgacctggacagtgga

SEQ ID NO: 178 - protein, 8 aa, Artificial Sequence
  Description of Artificial Sequence Synthetic peptide
AASDLDSG

SEQ ID NO: 179 - DNA, 24 bp, Artificial Sequence
  Description of Artificial Sequence Synthetic oligonucleotide
gctgcatctaccgacgacagtgga

SEQ ID NO: 180 - protein, 8 aa, Artificial Sequence
  Description of Artificial Sequence Synthetic peptide
AASTDDSG

SEQ ID NO: 181 - DNA, 24 bp, Artificial Sequence
  Description of Artificial Sequence Synthetic oligonucleotide
gctgcatctaccctggacgacgga

SEQ ID NO: 182 - protein, 8 aa, Artificial Sequence
  Description of Artificial Sequence Synthetic peptide
AASTLDDG

SEQ ID NO: 183 - DNA, 24 bp, Artificial Sequence
  Description of Artificial Sequence Synthetic oligonucleotide
gctgcatctaccctggacagtgac

SEQ ID NO: 184 - protein, 8 aa, Artificial Sequence
  Description of Artificial Sequence Synthetic peptide
AASTLDSD

SEQ ID NO: 185 - DNA, 27 bp, Artificial Sequence
  Description of Artificial Sequence Synthetic oligonucleotide
ctgcagtacgacaattatcctagaaca

SEQ ID NO: 186 - protein, 8 aa, Artificial Sequence
  Description of Artificial Sequence Synthetic peptide
QYDNYPRT

SEQ ID NO: 187 - DNA, 27 bp, Artificial Sequence
  Description of Artificial Sequence Synthetic oligonucleotide
ctgcagtacgccgactatcctagaaca

SEQ ID NO: 188 - protein, 8 aa, Artificial Sequence
  Description of Artificial Sequence Synthetic peptide
QYADYPRT

SEQ ID NO: 189 - DNA, 24 bp, Artificial Sequence
  Description of Artificial Sequence Synthetic oligonucleotide
ggatatatattcgactcctactat

SEQ ID NO: 190 - protein, 8 aa, Artificial Sequence
  Description of Artificial Sequence Synthetic peptide
GYIFDSYY

SEQ ID NO: 191 - DNA, 24 bp, Artificial Sequence
  Description of Artificial Sequence Synthetic oligonucleotide
ggatatatattcaccgactactat

SEQ ID NO: 192 - protein, 8 aa, Artificial Sequence
  Description of Artificial Sequence Synthetic peptide
GYIFDTYY

SEQ ID NO: 193 - DNA, 24 bp, Artificial Sequence
  Description of Artificial Sequence Synthetic oligonucleotide
atctaccctgacaacgtgaataca

SEQ ID NO: 194 - protein, 8 aa, Artificial Sequence
  Description of Artificial Sequence Synthetic peptide
IYPDNVNT

SEQ ID NO: 195 - DNA, 24 bp, Artificial Sequence
  Description of Artificial Sequence Synthetic oligonucleotide
atctaccctggggacgtgaataca

SEQ ID NO: 196 - protein, 8 aa, Artificial Sequence
  Description of Artificial Sequence Synthetic peptide
IYPGDVNT

SEQ ID NO: 197 - DNA, 24 bp, Artificial Sequence
  Description of Artificial Sequence Synthetic oligonucleotide
atctaccctgggaacgacaataca

SEQ ID NO: 198 - protein, 8 aa, Artificial Sequence
  Description of Artificial Sequence Synthetic peptide
IYPGNDNT

SEQ ID NO: 199 - DNA, 24 bp, Artificial Sequence
  Description of Artificial Sequence Synthetic oligonucleotide
atctaccctgggaacgtggacaca

SEQ ID NO: 200 - protein, 8 aa, Artificial Sequence
  Description of Artificial Sequence Synthetic peptide
IYPGNVDT

SEQ ID NO: 201 - DNA, 24 bp, Artificial Sequence
  Description of Artificial Sequence Synthetic oligonucleotide
atctaccctgggaacgtgaatgac

SEQ ID NO: 202 - protein, 8 aa, Artificial Sequence
  Description of Artificial Sequence Synthetic peptide
IYPGNVND

SEQ ID NO: 203 - DNA, 48 bp, Artificial Sequence
  Description of Artificial Sequence Synthetic oligonucleotide
tggatctaccctgggaacgtgaatacagactataacgagaagttcaaa

SEQ ID NO: 204 - protein, 16 aa, Artificial Sequence
  Description of Artificial Sequence Synthetic peptide
WIYPGNVNTDYNEKFK

SEQ ID NO: 205 - DNA, 27 bp, Artificial Sequence
  Description of Artificial Sequence Synthetic oligonucleotide
gctagaatggattacgacgcccactat

SEQ ID NO: 206 - protein, 9 aa, Artificial Sequence
  Description of Artificial Sequence Synthetic peptide
ARMDYDAHY

SEQ ID NO: 207 - protein, 467 aa, Artificial Sequence
  Description of Artificial Sequence Synthetic polypeptide
MACPGFLWALVISTCLEFSMAQVQLQQSGPEVKKPGASVKISCKASGYIFTSYYIQWVKHAPGQGLEWIGWIYPGNVNTKYNEKFKGKATLTADKSSSTAYMELSSLTSEDTAVYFCARMDYEAHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - DNA, 1407 bp, Artificial Sequence
  Description of Artificial Sequence Synthetic polynucleotide
atggcatgccctggcttcctgtgggcacttgtgatctccacctgtcttgaattttccatggctcaggtccagctgcagcagagcggtcccgaggtcaaaaaacccggcgcatccgtgaaaatcagttgtaaagcatccggttatatcttcacctcctactatatccagtgggtcaagcacgcacctggtcagggactggaatggatcggatggatctaccctgggaacgtgaatacaaagtataacgagaagttcaaaggcaaggctacactgactgcagacaagtccagctctactgcatacatggagctgagttcactgactagcgaagacaccgccgtgtatttctgcgctagaatggattacgaagcccactattggggacaggggaccacagtcaccgtgtcctccgccagcacaaaaggtccttccgtgttccctctggcaccatctagtaagtctacaagtggcggaactgccgctctgggctgtctggtgaaggattacttccctgagccagtcaccgtgtcctggaacagcggtgcactgacttctggcgtccatacctttccagccgtgctgcagtcatccggactgtactccctgagctctgtggtcactgtccccagttcatccctggggacccagacatatatctgcaacgtgaatcacaaaccttctaatacaaaggtcgacaagaaagtggaaccaaaatcctgtgataagactcatacctgcccaccttgtccagctcctgagctgctgggaggtccaagcgtgttcctgtttccacccaaacccaaggacaccctgatgattagccggacccctgaagtcacatgcgtggtcgtggacgtgtctcacgaggatccagaagtcaagttcaactggtacgtggatggcgtcgaggtgcataatgccaaaaccaagccacgagaggaacagtacaacagtacatatcgtgtcgtgtcagtcctgactgtgctgcaccaggactggctgaacggaaaggagtataaatgcaaggtgtccaacaaggccctgccagcccccatcgagaagaccattagcaaagctaaggggcagcccagggaacctcaggtgtacacactgcctccaagtcgggacgagctgactaaaaaccaggtcagcctgacctgtctggtgaagggtttttatccaagcgatatcgcagtggagtgggaatctaatggccagcccgagaacaattacaagactaccccccctgtgctggactctgatggtagtttctttctgtattctaaactgaccgtggataagagtaggtggcagcagggcaacgtcttctcatgctccgtgatgcatgaagctctgcacaatcattacacccagaaaagcctgtctctgagtcctggaaagtgataa

SEQ ID NO: 209 - protein, 467 aa, Artificial Sequence
  Description of Artificial Sequence Synthetic polypeptide
MACPGFLWALVISTCLEFSMAQVQLQQSGPEVKKPGASVKISCKASGYIFTSYYIQWVIHRPGQGLEWIGWIYPGNVNTKYNEKFKGKATLTADKSSSTAYMELSSLTSEDTAVYFCARMDYEAHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - DNA, 1407 bp, Artificial Sequence
  Description of Artificial Sequence Synthetic polynucleotide
atggcatgccctggcttcctgtgggcacttgtgatctccacctgtcttgaattttccatggctcaggtccagctgcagcagagcggtcccgaggtcaaaaaacccggcgcatccgtgaaaatcagttgtaaagcatccggttatatcttcacctcctactatatccagtgggtcatccacaggcctggtcagggactggaatggatcggatggatctaccctgggaacgtgaatacaaagtataacgagaagttcaaaggcaaggctacactgactgcagacaagtccagctctactgcatacatggagctgagttcactgactagcgaagacaccgccgtgtatttctgcgctagaatggattacgaagcccactattggggacaggggaccacagtcaccgtgtcctccgccagcacaaaaggtccttccgtgttccctctggcaccatctagtaagtctacaagtggcggaactgccgctctgggctgtctggtgaaggattacttccctgagccagtcaccgtgtcctggaacagcggtgcactgacttctggcgtccatacctttccagccgtgctgcagtcatccggactgtactccctgagctctgtggtcactgtccccagttcatccctggggacccagacatatatctgcaacgtgaatcacaaaccttctaatacaaaggtcgacaagaaagtggaaccaaaatcctgtgataagactcatacctgcccaccttgtccagctcctgagctgctgggaggtccaagcgtgttcctgtttccacccaaacccaaggacaccctgatgattagccggacccctgaagtcacatgcgtggtcgtggacgtgtctcacgaggatccagaagtcaagttcaactggtacgtggatggcgtcgaggtgcataatgccaaaaccaagccacgagaggaacagtacaacagtacatatcgtgtcgtgtcagtcctgactgtgctgcaccaggactggctgaacggaaaggagtataaatgcaaggtgtccaacaaggccctgccagcccccatcgagaagaccattagcaaagctaaggggcagcccagggaacctcaggtgtacacactgcctccaagtcgggacgagctgactaaaaaccaggtcagcctgacctgtctggtgaagggtttttatccaagcgatatcgcagtggagtgggaatctaatggccagcccgagaacaattacaagactaccccccctgtgctggactctgatggtagtttctttctgtattctaaactgaccgtggataagagtaggtggcagcagggcaacgtcttctcatgctccgtgatgcatgaagctctgcacaatcattacacccagaaaagcctgtctctgagtcctggaaagtgataa

SEQ ID NO: 211 - protein, 467 aa, Artificial Sequence
  Description of Artificial Sequence Synthetic polypeptide
MACPGFLWALVISTCLEFSMAQVQLQQSGPEVKKPGASVKISCKASGYIFTSYYIQWVKQAPGQGLEWIGWIYPGNVNTKYNEKFKGKATLTADKSSSTAYMELSSLTSEDTAVYFCARMDYEAHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - DNA, 1407 bp, Artificial Sequence
  Description of Artificial Sequence Synthetic polynucleotide
atggcatgccctggcttcctgtgggcacttgtgatctccacctgtcttgaattttccatggctcaggtccagctgcagcagagcggtcccgaggtcaaaaaacccggcgcatccgtgaaaatcagttgtaaagcatccggttatatcttcacctcctactatatccagtgggtcaagcaggcacctggtcagggactggaatggatcggatggatctaccctgggaacgtgaatacaaagtataacgagaagttcaaaggcaaggctacactgactgcagacaagtccagctctactgcatacatggagctgagttcactgactagcgaagacaccgccgtgtatttctgcgctagaatggattacgaagcccactattggggacaggggaccacagtcaccgtgtcctccgccagcacaaaaggtccttccgtgttccctctggcaccatctagtaagtctacaagtggcggaactgccgctctgggctgtctggtgaaggattacttccctgagccagtcaccgtgtcctggaacagcggtgcactgacttctggcgtccatacctttccagccgtgctgcagtcatccggactgtactccctgagctctgtggtcactgtccccagttcatccctggggacccagacatatatctgcaacgtgaatcacaaaccttctaatacaaaggtcgacaagaaagtggaaccaaaatcctgtgataagactcatacctgcccaccttgtccagctcctgagctgctgggaggtccaagcgtgttcctgtttccacccaaacccaaggacaccctgatgattagccggacccctgaagtcacatgcgtggtcgtggacgtgtctcacgaggatccagaagtcaagttcaactggtacgtggatggcgtcgaggtgcataatgccaaaaccaagccacgagaggaacagtacaacagtacatatcgtgtcgtgtcagtcctgactgtgctgcaccaggactggctgaacggaaaggagtataaatgcaaggtgtccaacaaggccctgccagcccccatcgagaagaccattagcaaagctaaggggcagcccagggaacctcaggtgtacacactgcctccaagtcgggacgagctgactaaaaaccaggtcagcctgacctgtctggtgaagggtttttatccaagcgatatcgcagtggagtgggaatctaatggccagcccgagaacaattacaagactaccccccctgtgctggactctgatggtagtttctttctgtattctaaactgaccgtggataagagtaggtggcagcagggcaacgtcttctcatgctccgtgatgcatgaagctctgcacaatcattacacccagaaaagcctgtctctgagtcctggaaagtgataa

SEQ ID NO: 213 - protein, 467 aa, Artificial Sequence
  Description of Artificial Sequence Synthetic polypeptide
MACPGFLWALVISTCLEFSMAQVQLQQSGPEVKKPGASVKISCKASGYIFTSYYIQWVIQRPGQGLEWIGWIYPGNVNTKYNEKFKGKATLTADKSSSTAYMELSSLTSEDTAVYFCARMDYEAHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - DNA, 1407 bp, Artificial Sequence
  Description of Artificial Sequence Synthetic polynucleotide
atggcatgccctggcttcctgtgggcacttgtgatctccacctgtcttgaattttccatggctcaggtccagctgcagcagagcggtcccgaggtcaaaaaacccggcgcatccgtgaaaatcagttgtaaagcatccggttatatcttcacctcctactatatccagtgggtcatccagaggcctggtcagggactggaatggatcggatggatctaccctgggaacgtgaatacaaagtataacgagaagttcaaaggcaaggctacactgactgcagacaagtccagctctactgcatacatggagctgagttcactgactagcgaagacaccgccgtgtatttctgcgctagaatggattacgaagcccactattggggacaggggaccacagtcaccgtgtcctccgccagcacaaaaggtccttccgtgttccctctggcaccatctagtaagtctacaagtggcggaactgccgctctgggctgtctggtgaaggattacttccctgagccagtcaccgtgtcctggaacagcggtgcactgacttctggcgtccatacctttccagccgtgctgcagtcatccggactgtactccctgagctctgtggtcactgtccccagttcatccctggggacccagacatatatctgcaacgtgaatcacaaaccttctaatacaaaggtcgacaagaaagtggaaccaaaatcctgtgataagactcatacctgcccaccttgtccagctcctgagctgctgggaggtccaagcgtgttcctgtttccacccaaacccaaggacaccctgatgattagccggacccctgaagtcacatgcgtggtcgtggacgtgtctcacgaggatccagaagtcaagttcaactggtacgtggatggcgtcgaggtgcataatgccaaaaccaagccacgagaggaacagtacaacagtacatatcgtgtcgtgtcagtcctgactgtgctgcaccaggactggctgaacggaaaggagtataaatgcaaggtgtccaacaaggccctgccagcccccatcgagaagaccattagcaaagctaaggggcagcccagggaacctcaggtgtacacactgcctccaagtcgggacgagctgactaaaaaccaggtcagcctgacctgtctggtgaagggtttttatccaagcgatatcgcagtggagtgggaatctaatggccagcccgagaacaattacaagactaccccccctgtgctggactctgatggtagtttctttctgtattctaaactgaccgtggataagagtaggtggcagcagggcaacgtcttctcatgctccgtgatgcatgaagctctgcacaatcattacacccagaaaagcctgtctctgagtcctggaaagtgataa

SEQ ID NO: 215 - protein, 467 aa, Artificial Sequence
  Description of Artificial Sequence Synthetic polypeptide
MACPGFLWALVISTCLEFSMAQVQLQQSGPEVKKPGASVKISCKASGYIFTSYYIQWVKQRPGQGLEWIGWIYPGNVNTKYNEKFKGKATLTADKSSSTAYMELSSLTSEDTAVYFCARMDYEAHYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - DNA, 1407 bp, Artificial Sequence
  Description of Artificial Sequence Synthetic polynucleotide
atggcatgccctggcttcctgtgggcacttgtgatctccacctgtcttgaattttccatggctcaggtccagctgcagcagagcggtcccgaggtcaaaaaacccggcgcatccgtgaaaatcagttgtaaagcatccggttatatcttcacctcctactatatccagtgggtcaagcagaggcctggtcagggactggaatggatcggatggatctaccctgggaacgtgaatacaaagtataacgagaagttcaaaggcaaggctacactgactgcagacaagtccagctctactgcatacatggagctgagttcactgactagcgaagacaccgccgtgtatttctgcgctagaatggattacgaagcccactattggggacaggggaccacagtcaccgtgtcctccgccagcacaaaaggtccttccgtgttccctctggcaccatctagtaagtctacaagtggcggaactgccgctctgggctgtctggtgaaggattacttccctgagccagtcaccgtgtcctggaacagcggtgcactgacttctggcgtccatacctttccagccgtgctgcagtcatccggactgtactccctgagctctgtggtcactgtccccagttcatccctggggacccagacatatatctgcaacgtgaatcacaaaccttctaatacaaaggtcgacaagaaagtggaaccaaaatcctgtgataagactcatacctgcccaccttgtccagctcctgagctgctgggaggtccaagcgtgttcctgtttccacccaaacccaaggacaccctgatgattagccggacccctgaagtcacatgcgtggtcgtggacgtgtctcacgaggatccagaagtcaagttcaactggtacgtggatggcgtcgaggtgcataatgccaaaaccaagccacgagaggaacagtacaacagtacatatcgtgtcgtgtcagtcctgactgtgctgcaccaggactggctgaacggaaaggagtataaatgcaaggtgtccaacaaggccctgccagcccccatcgagaagaccattagcaaagctaaggggcagcccagggaacctcaggtgtacacactgcctccaagtcgggacgagctgactaaaaaccaggtcagcctgacctgtctggtgaagggtttttatccaagcgatatcgcagtggagtgggaatctaatggccagcccgagaacaattacaagactaccccccctgtgctggactctgatggtagtttctttctgtattctaaactgaccgtggataagagtaggtggcagcagggcaacgtcttctcatgctccgtgatgcatgaagctctgcacaatcattacacccagaaaagcctgtctctgagtcctggaaagtgataa

SEQ ID NO: 217 - protein, 116 aa, Artificial Sequence
  Description of Artificial Sequence Synthetic polypeptide
QVQLQQSGPEVKKPGASVKISCKASGYIFTSYYIQWVKHRPGQGLEWIGWIYPGNVNTKYNEKFKGKATLTADKSSSTAYMELSSLTSEDTAVYFCARMDYEAHYWGQGTTVTVSS

SEQ ID NO: 218 - protein, 116 aa, Artificial Sequence
  Description of Artificial Sequence Synthetic polypeptide
QVQLQQSGPEVKKPGASVKISCKASGYIFTSYYIQWVIHAPGQGLEWIAWIYPGNVNTKYNEKFKGKATLTADKSSSTAYMELSSLTSEDTAVYFCARMDYEAHYWGQGTTVTVSS

SEQ ID NO: 219 - protein, 116 aa, Artificial Sequence
  Description of Artificial Sequence Synthetic polypeptide
QVQLQQSGPEVVKPGASVKISCKASGYIFTSYYIQWVIHAPGQGLEWIGWIYPGNVNTKYNEKFKGKATLTADKSSSTAYMELSSLTSEDTAVYFCARMDYEAHYWGQGTTVTVSS

SEQ ID NO: 220 - protein, 116 aa, Artificial Sequence
  Description of Artificial Sequence Synthetic polypeptide
QVQLQQSGPEVKKPGASVKISCKASGYIFTSYYIQWVIHAPGQGLEWVAYIYPGNVNTKYNEKFKGKATLTADKSSSTAYMELSSLTSEDTAVYFCARMDYEAHYWGQGTTVTVSS

SEQ ID NO: 221 - protein, 116 aa, Artificial Sequence
  Description of Artificial Sequence Synthetic polypeptide
QVQLQQSGPEVKKPGASVKISCKASGYIFTSYYIQWVIHAPGQGLEWVAWIYPGNVNTKYNEKFKGKATLTADKSSSTAYMELSSLTSEDTAVYFCARMDYEAHYWGQGTTVTVSS

SEQ ID NO: 222 - DNA, 33 bp, Artificial Sequence
  Description of Artificial Sequence Synthetic primer
gtgacaatcacttgtgacgcttcccaggaaatt

SEQ ID NO: 223 - DNA, 33 bp, Artificial Sequence
  Description of Artificial Sequence Synthetic primer
aatttcctgggaagcgtcacaagtgattgtcac

SEQ ID NO: 224 - DNA, 33 bp, Artificial Sequence
  Description of Artificial Sequence Synthetic primer
atcacttgtcgcgctgaccaggaaattagcgga

SEQ ID NO: 225 - DNA, 33 bp, Artificial Sequence
  Description of Artificial Sequence Synthetic primer
tccgctaatttcctggtcagcgcgacaagtgat

SEQ ID NO: 226 - DNA, 33 bp, Artificial Sequence
  Description of Artificial Sequence Synthetic primer
acttgtcgcgcttccgacgaaattagcggatac

SEQ ID NO: 227 - DNA, 33 bp, Artificial Sequence
  Description of Artificial Sequence Synthetic primer
gtatccgctaatttcgtcggaagcgcgacaagt

SEQ ID NO: 228 - DNA, 33 bp, Artificial Sequence
  Description of Artificial Sequence Synthetic primer
tgtcgcgcttcccaggacattagcggatacctg

SEQ ID NO: 229 - DNA, 33 bp, Artificial Sequence
  Description of Artificial Sequence Synthetic primer
caggtatccgctaatgtcctgggaagcgcgaca

SEQ ID NO: 230 - DNA, 33 bp, Artificial Sequence
  Description of Artificial Sequence Synthetic primer
gcttcccaggaaattgacggatacctgacttgg

SEQ ID NO: 231 - DNA, 33 bp, Artificial Sequence
  Description of Artificial Sequence Synthetic primer
ccaagtcaggtatccgtcaatttcctgggaagc

SEQ ID NO: 232 - DNA, 33 bp, Artificial Sequence
  Description of Artificial Sequence Synthetic primer
aagcgactgatctacgacgcatctaccctggac

SEQ ID NO: 233 - DNA, 33 bp, Artificial Sequence
  Description of Artificial Sequence Synthetic primer
gtccagggtagatgcgtcgtagatcagtcgctt

SEQ ID NO: 234 - DNA, 33 bp, Artificial Sequence
  Description of Artificial Sequence Synthetic primer
ctgatctacgctgcagacaccctggacagtgga

SEQ ID NO: 235 - DNA, 33 bp, Artificial Sequence
  Description of Artificial Sequence Synthetic primer
tccactgtccagggtgtctgcagcgtagatcag

SEQ ID NO: 236 - DNA, 33 bp, Artificial Sequence
  Description of Artificial Sequence Synthetic primer
atctacgctgcatctgacctggacagtggagtg

SEQ ID NO: 237 - DNA, 33 bp, Artificial Sequence
  Description of Artificial Sequence Synthetic primer
cactccactgtccaggtcagatgcagcgtagat

SEQ ID NO: 238 - DNA, 33 bp, Artificial Sequence
  Description of Artificial Sequence Synthetic primer
tacgctgcatctaccgacgacagtggagtgcct

SEQ ID NO: 239 - DNA, 33 bp, Artificial Sequence
  Description of Artificial Sequence Synthetic primer
aggcactccactgtcgtcggtagatgcagcgta

SEQ ID NO: 240 - DNA, 33 bp, Artificial Sequence
  Description of Artificial Sequence Synthetic primer
gcatctaccctggacgacggagtgcctaagagg

SEQ ID NO: 241 - DNA, 33 bp, Artificial Sequence
  Description of Artificial Sequence Synthetic primer
cctcttaggcactccgtcgtccagggtagatgc

SEQ ID NO: 242 - DNA, 33 bp, Artificial Sequence
  Description of Artificial Sequence Synthetic primer
tctaccctggacagtgacgtgcctaagaggttc

SEQ ID NO: 243 - DNA, 33 bp, Artificial Sequence
  Description of Artificial Sequence Synthetic primer
gaacctcttaggcacgtcactgtccagggtaga

SEQ ID NO: 244 - DNA, 33 bp, Artificial Sequence
  Description of Artificial Sequence Synthetic primer
aactgcctgcagtacgacaattatcctagaaca

SEQ ID NO: 245 - DNA, 33 bp, Artificial Sequence
  Description of Artificial Sequence Synthetic primer
tgttctaggataattgtcgtactgcaggcagtt

SEQ ID NO: 246 - DNA, 33 bp, Artificial Sequence
  Description of Artificial Sequence Synthetic primer
tgcctgcagtacgccgactatcctagaacattt

SEQ ID NO: 247 - DNA, 33 bp, Artificial Sequence
  Description of Artificial Sequence Synthetic primer
aaatgttctaggatagtcggcgtactgcaggca

SEQ ID NO: 248 - DNA, 33 bp, Artificial Sequence
  Description of Artificial Sequence Synthetic primer
tccggttatatcttcgactcctactatatccag

SEQ ID NO: 249 - DNA, 33 bp, Artificial Sequence
  Description of Artificial Sequence Synthetic primer
ctggatatagtaggagtcgaagatataaccgga

SEQ ID NO: 250 - DNA, 33 bp, Artificial Sequence
  Description of Artificial Sequence Synthetic primer
ggttatatcttcaccgactactatatccagtgg

SEQ ID NO: 251 - DNA, 33 bp, Artificial Sequence
  Description of Artificial Sequence Synthetic primer
ccactggatatagtagtcggtgaagatataacc

SEQ ID NO: 252 - DNA, 33 bp, Artificial Sequence
  Description of Artificial Sequence Synthetic primer
ggatggatctaccctgacaacgtgaatacaaag

SEQ ID NO: 253 - DNA, 33 bp, Artificial Sequence
  Description of Artificial Sequence Synthetic primer
ctttgtattcacgttgtcagggtagatccatcc

SEQ ID NO: 254 - DNA, 33 bp, Artificial Sequence
  Description of Artificial Sequence Synthetic primer
tggatctaccctggggacgtgaatacaaagtat

SEQ ID NO: 255 - DNA, 33 bp, Artificial Sequence
  Description of Artificial Sequence Synthetic primer
atactttgtattcacgtccccagggtagatcca

SEQ ID NO: 256 - DNA, 33 bp, Artificial Sequence
  Description of Artificial Sequence Synthetic primer
atctaccctgggaacgacaatacaaagtataac

SEQ ID NO: 257 - DNA, 33 bp, Artificial Sequence
  Description of Artificial Sequence Synthetic primer
gttatactttgtattgtcgttcccagggtagat

SEQ ID NO: 258 - DNA, 33 bp, Artificial Sequence
  Description of Artificial Sequence Synthetic primer
taccctgggaacgtggacacaaagtataacgag

SEQ ID NO: 259 - DNA, 33 bp, Artificial Sequence
  Description of Artificial Sequence Synthetic primer
ctcgttatactttgtgtccacgttcccagggta

SEQ ID NO: 260 - DNA, 33 bp, Artificial Sequence
  Description of Artificial Sequence Synthetic primer
cctgggaacgtgaatgacaagtataacgagaag

SEQ ID NO: 261 - DNA, 33 bp, Artificial Sequence
  Description of Artificial Sequence Synthetic primer
cttctcgttatacttgtcattcacgttcccagg

SEQ ID NO: 262 - DNA, 33 bp, Artificial Sequence
  Description of Artificial Sequence Synthetic primer
gggaacgtgaatacagactataacgagaagttc

SEQ ID NO: 263 - DNA, 33 bp, Artificial Sequence
  Description of Artificial Sequence Synthetic primer
gaacttctcgttatagtctgtattcacgttccc

SEQ ID NO: 264 - DNA, 33 bp, Artificial Sequence
  Description of Artificial Sequence Synthetic primer
gctagaatggattacgacgcccactattgggga

SEQ ID NO: 265 - DNA, 33 bp, Artificial Sequence
  Description of Artificial Sequence Synthetic primer
tccccaatagtgggcgtcgtaatccattctagc

SEQ ID NO: 266 - DNA, 33 bp, Artificial Sequence
  Description of Artificial Sequence Synthetic primer
gtgacaatcacttgttacgcttcccaggaaatt

SEQ ID NO: 267 - DNA, 33 bp, Artificial Sequence
  Description of Artificial Sequence Synthetic primer
aatttcctgggaagcgtaacaagtgattgtcac

SEQ ID NO: 268 - DNA, 33 bp, Artificial Sequence
  Description of Artificial Sequence Synthetic primer
atcacttgtcgcgcttaccaggaaattagcgga

SEQ ID NO: 269 - DNA, 33 bp, Artificial Sequence
  Description of Artificial Sequence Synthetic primer
tccgctaatttcctggtaagcgcgacaagtgat

SEQ ID NO: 270 - DNA, 33 bp, Artificial Sequence
  Description of Artificial Sequence Synthetic primer
acttgtcgcgcttcctacgaaattagcggatac

SEQ ID NO: 271 - DNA, 33 bp, Artificial Sequence
  Description of Artificial Sequence Synthetic primer
gtatccgctaatttcgtaggaagcgcgacaagt

SEQ ID NO: 272 - DNA, 33 bp, Artificial Sequence
  Description of Artificial Sequence Synthetic primer
tgtcgcgcttcccagtacattagcggatacctg

SEQ ID NO: 273 - DNA, 33 bp, Artificial Sequence
  Description of Artificial Sequence Synthetic primer
caggtatccgctaatgtactgggaagcgcgaca

SEQ ID NO: 274 - DNA, 33 bp, Artificial Sequence
  Description of Artificial Sequence Synthetic primer
gcttcccaggaaatttacggatacctgacttgg

SEQ ID NO: 275 - DNA, 33 bp, Artificial Sequence
  Description of Artificial Sequence Synthetic primer
ccaagtcaggtatccgtaaatttcctgggaagc

SEQ ID NO: 276 - DNA, 33 bp, Artificial Sequence
  Description of Artificial Sequence Synthetic primer
aagcgactgatctactacgcatctaccctggac

SEQ ID NO: 277 - DNA, 33 bp, Artificial Sequence
  Description of Artificial Sequence Synthetic primer
gtccagggtagatgcgtagtagatcagtcgctt

SEQ ID NO: 278 - DNA, 33 bp, Artificial Sequence
  Description of Artificial Sequence Synthetic primer
ctgatctacgctgcatacaccctggacagtgga

SEQ ID NO: 279 - DNA, 33 bp, Artificial Sequence
  Description of Artificial Sequence Synthetic primer
tccactgtccagggtgtatgcagcgtagatcag

SEQ ID NO: 280 - DNA, 33 bp, Artificial Sequence
  Description of Artificial Sequence Synthetic primer
atctacgctgcatcttacctggacagtggagtg

SEQ ID NO: 281 - DNA, 33 bp, Artificial Sequence
  Description of Artificial Sequence Synthetic primer
cactccactgtccaggtaagatgcagcgtagat

SEQ ID NO: 282 - DNA, 33 bp, Artificial Sequence
  Description of Artificial Sequence Synthetic primer
tacgctgcatctacctacgacagtggagtgcct

SEQ ID NO: 283 - DNA, 33 bp, Artificial Sequence
  Description of Artificial Sequence Synthetic primer
aggcactccactgtcgtaggtagatgcagcgta

SEQ ID NO: 284 - DNA, 33 bp, Artificial Sequence
  Description of Artificial Sequence Synthetic primer
gcatctaccctggactacggagtgcctaagagg

SEQ ID NO: 285 - DNA, 33 bp, Artificial Sequence
  Description of Artificial Sequence Synthetic primer
cctcttaggcactccgtagtccagggtagatgc

SEQ ID NO: 286 - DNA, 33 bp, Artificial Sequence
  Description of Artificial Sequence Synthetic primer
tctaccctggacagttacgtgcctaagaggttc

SEQ ID NO: 287 - DNA, 33 bp, Artificial Sequence
  Description of Artificial Sequence Synthetic primer
gaacctcttaggcacgtaactgtccagggtaga

SEQ ID NO: 288 - DNA, 33 bp, Artificial Sequence
  Description of Artificial Sequence Synthetic primer
aactgcctgcagtactacaattatcctagaaca

SEQ ID NO: 289 - DNA, 33 bp, Artificial Sequence
  Description of Artificial Sequence Synthetic primer
tgttctaggataattgtagtactgcaggcagtt

SEQ ID NO: 290 - DNA, 33 bp, Artificial Sequence
  Description of Artificial Sequence Synthetic primer
tgcctgcagtacgcctactatcctagaacattt

SEQ ID NO: 291 - DNA, 33 bp, Artificial Sequence
  Description of Artificial Sequence Synthetic primer
aaatgttctaggatagtaggcgtactgcaggca

SEQ ID NO: 292 - DNA, 33 bp, Artificial Sequence
  Description of Artificial Sequence Synthetic primer
tccggttatatcttctactcctactatatccag

SEQ ID NO: 293 - DNA, 33 bp, Artificial Sequence
  Description of Artificial Sequence Synthetic primer
ctggatatagtaggagtagaagatataaccgga

SEQ ID NO: 294 - DNA, 33 bp, Artificial Sequence
  Description of Artificial Sequence Synthetic primer
ggttatatcttcacctactactatatccagtgg

SEQ ID NO: 295 - DNA, 33 bp, Artificial Sequence
  Description of Artificial Sequence Synthetic primer
ccactggatatagtagtaggtgaagatataacc

SEQ ID NO: 296 - DNA, 33 bp, Artificial Sequence
  Description of Artificial Sequence Synthetic primer
ggatggatctacccttacaacgtgaatacaaag

SEQ ID NO: 297 - DNA, 33 bp, Artificial Sequence
  Description of Artificial Sequence Synthetic primer
ctttgtattcacgttgtaagggtagatccatcc

SEQ ID NO: 298 - DNA, 33 bp, Artificial Sequence
  Description of Artificial Sequence Synthetic primer
tggatctaccctgggtacgtgaatacaaagtat

SEQ ID NO: 299 - DNA, 33 bp, Artificial Sequence
  Description of Artificial Sequence Synthetic primer
atactttgtattcacgtacccagggtagatcca

SEQ ID NO: 300 - DNA, 33 bp, Artificial Sequence
  Description of Artificial Sequence Synthetic primer
atctaccctgggaactacaatacaaagtataac

SEQ ID NO: 301 - DNA, 33 bp, Artificial Sequence
  Description of Artificial Sequence Synthetic primer
gttatactttgtattgtagttcccagggtagat

SEQ ID NO: 302 - DNA, 33 bp, Artificial Sequence
  Description of Artificial Sequence Synthetic primer
taccctgggaacgtgtacacaaagtataacgag

SEQ ID NO: 303 - DNA, 33 bp, Artificial Sequence
  Description of Artificial Sequence Synthetic primer
ctcgttatactttgtgtacacgttcccagggta

SEQ ID NO: 304 - DNA, 33 bp, Artificial Sequence
  Description of Artificial Sequence Synthetic primer
cctgggaacgtgaattacaagtataacgagaag

SEQ ID NO: 305 - DNA, 33 bp, Artificial Sequence
  Description of Artificial Sequence Synthetic primer
cttctcgttatacttgtaattcacgttcccagg

SEQ ID NO: 306 - DNA, 33 bp, Artificial Sequence
  Description of Artificial Sequence Synthetic primer
gggaacgtgaatacatactataacgagaagttc

SEQ ID NO: 307 - DNA, 33 bp, Artificial Sequence
  Description of Artificial Sequence Synthetic primer
gaacttctcgttatagtatgtattcacgttccc

SEQ ID NO: 308 - DNA, 33 bp, Artificial Sequence
  Description of Artificial Sequence Synthetic primer
gctagaatggattactacgcccactattgggga

SEQ ID NO: 309 - DNA, 33 bp, Artificial Sequence
  Description of Artificial Sequence Synthetic primer
tccccaatagtgggcgtagtaatccattctagc

(19) United States
(12) Patent Application Publication
(10) Pub. No.: US 2017/0008955 A1
(43) Pub. Date: Jan. 12, 2017

(54) HUMANIZED BETA-AMYLOID BINDING MOLECULES AND USES THEREOF

(71) Applicant: CNJ HOLDINGS, INC., WINNIPEG, MANITOBA (CA)

(72) Inventors: Cory Nykiforuk, Winnipeg (CA); Darrell Johnstone, Winnipeg (CA); Neil Cashman, Vancouver (CA)

(21) Appl. No.: 15/115,296

(22) PCT Filed: Feb. 3, 2015
(86) PCT No.: PCT/CA2015/050079

§ 371 Date: Jul. 29, 2016

Related U.S. Application Data
(60) Provisional application No. 61/935,314, filed on Feb. 3, 2014.
(60) Provisional application No. 62/025,465, filed on Jul. 16, 2014.

(51) Int. Cl. C07K 16/18; A61K 39/395

(52) U.S. Cl. CPC C07K 16/18; A61K 39/3955; C07K 2317/24; C07K 2317/565; C07K 2317/55; C07K 2317/54; C07K 2317/624; C07K 2317/622; C07K 2317/92; C07K 2317/34

(57) ABSTRACT

Provided herein are humanized antibodies or antigen-binding fragments thereof, that can bind to a cyclic peptide comprising the amino acid sequence SNK, wherein the K (Lysine) is solvent-accessible and methods of treating and/or preventing amyloid-beta associated diseases such as Alzheimer's disease.

### RELATED APPLICATIONS

This is related to U.S. Provisional Application No. 61/935,314, filed Feb. 3, 2014, and U.S. Provisional Application No. 62/025,465, filed Jul. 16, 2014, both of which are incorporated herein in their entireties.

### BACKGROUND

Alzheimer's disease (AD) is a common dementing (disordered memory and cognition) neurodegenerative disease. It is associated with accumulation in the brain of extracellular plaques composed predominantly of the amyloid beta peptides (also referred to as amyloid β, Abeta or Aβ), including but not limited to Aβ (1-40), Aβ (1-42) and Aβ (1-43) peptides. These Aβ peptides are proteolytic products of amyloid precursor protein (APP). In addition, neurofibrillary tangles, composed principally of abnormally phosphorylated tau protein (a neuronal microtubule-associated protein), accumulate intracellularly in dying neurons. The Aβ (1-42) is the dominant species in the amyloid plaques of Alzheimer's disease patients. Aβ oligomerization has been shown to be a key part of neurotoxicity in Alzheimer's disease (Tu et al., Oligomeric Aβ-induced synaptic dysfunction in Alzheimer's disease, Mol Nerurodegen, 2014, 9(48); Jack et al., Biomarker modeling of Alzheimer's disease, Neuron, 2013, 80(6): 1347-58; Vos et al., Prediction of Alzheimer disease in subjects with amnestic and nonamnestic MCI, Neurology, 2013, 80(12):1124-32; Shankar and Walsh, Alzheimer's disease: synaptic dysfunction and Abeta, Mol Neurodegener, 2009, 4(48); Reed et al., Cognitive effects of cell-derived and synthetically derived Aβ oligomers, Neurobiol Aging, 2011, 32(10): 1784-94; Roher et al., 1993, J Neurochem 61:1916-26; McLean et al., Soluble pool of Abeta amyloid as determinant of severity of neurodegeneration in Alzheimer's disease, Ann Neurol, 1999, 46(6): 860-6; Lue et al., Soluble amyloid beta peptide concentration as predictor of synaptic change in Alzheimer's disease, Am J Pathol, 1999, 155(3):853-62; Naslund et al., Correlation between elevated levels of amyloid beta-peptide in the brain and cognitive decline, JAMA, 2000, 283(12):1574-7; De Felice et al., Alzheimer's disease-type neuronal tau hyperphosphorylation induced by A beta oligomers, Neurobiol Aging, 2008, 29(9): 1334-47; Selkoe D J, Resolving controversies on the path to Alzheimer's therapeutics, Nature Med, 2011, 17(9):1060-65; Vossel et al., Tau reduction prevents Abeta-induced defects in axonal transport, Science, 2010, 330:198-; Beninolva et al., The toxic Aβ oligomer and Alzheimer's disease: an emperor in need of clothes, Nature Neurosci, 2012, 15(3): 349-357).

It has been found that a particular molecular species of Aβ, in which the peptide is oligomerized, mediates the major component of neurotoxicity observed in Alzheimer's disease and mouse models of the disease (Walsh et al., Naturally secreted oligomers of amyloid beta protein potently inhibit hippocampal long-term potentiation in vivo, Nature, 2002, 416(6880): 535-9). Aβ oligomer toxicity can be manifested by dysfunction of neuronal insulin receptors (Zhao et al., Amyloid beta oligomers induce impairment of neuronal insulin receptors, FASEB J. 2008, 22(1):246-60), and by interference with normal synaptic function, particularly in the hippocampus, by ectopic activation of glutamatergic receptors (De Felice et al., 2007. Abeta oligomers induce neuronal oxidative stress through an N-methyl-D-aspartate receptor-dependent mechanism that is blocked by the Alzheimer drug memantine, J. Biol. Chem. 282:11590-11601; Nimmrich et al., Amyloid beta oligomers (Abeta(1-42) globulomer) suppress spontaneous synaptic activity by inhibition of P/Q-type calcium currents, J Neurosci., 2008, 23; 28(4):788-97).

The aberrant cleavage of the amyloid precursor protein (APP) by beta-secretase and then gamma-secretase, results in the formation of peptide Aβ (1-42). The monomeric peptides undergo a conformational change during rapid assembly of soluble, toxic AP oligomers, which eventually further aggregate to form the insoluble amyloid plaques that are one of the pathological hallmarks of Alzheimer's disease. Although a large number of cerebral amyloid plaques are usually associated with Alzheimer's disease, cognitive loss has been found to correlate poorly with the number of amyloid plaques. Instead, cognitive loss has been found to more reliably correlate with other forms of Aβ, for example soluble AP oligomers or aggregates, suggesting that Aβ oligomers might be more directly linked to neuronal and synaptic loss. In healthy individuals, antibodies specific to Aβ(1-42) are naturally present. It was reported that the concentration of antibodies against the oligomeric forms of Aβ(1-42) in particular declined with age and in advanced Alzheimer's disease (Britschgi et al., Neuroprotective natural antibodies to assemblies of amyloidogenic peptides decrease with normal aging and advancing Alzheimer's disease. Proc. Natl. Acad. Sci. USA 2009, 106(29):12145-50).

Many in vitro and in vivo studies have been conducted and the results demonstrate that immune therapy against Aβ can lead to the improvement of both the pathology and behavior of transgenic mice expressing human mutant APP (Hamley I W, 2012 Chem Rev 112:5147-5192). Unfortunately, these positive immunotherapy results in mice have not translated well in humans as there were adverse events associated with the treatment, including autoimmune meningoencephalitis, and appearance of amyloid-related imaging abnormalities (ARIAs; both vasogenic edema, ARIA-e and microhemorrhage, ARIA-H) during clinical trials (Gilman et al., Clinical effects of Abeta immunization (AN1792) in patients with AD in an interrupted trial, Neurology, 2005, 10; 64(9):1553-62). (Liu et al 2012 Nat Rev Neurol 8:465; Schenk et al 2004, Curr Opin Immunol 16)(5):599-606; Schenk 2002, Nat Rev Neurosci 3(10):824-828). To date, there are still numerous immunotherapy treatments, both passive and active (Lannfelt et al., Amyloid-β-directed immunotherapy for Alzheimer's disease,JIntern Med, 2014, 275:284-95; Lannfelt et al., Perspectives on future Alzheimer therapies: amyloid-β protofibrils—a new target for immunotherapy with BAN2401 in Alzheimer's disease, Alzherimer's Research & Therapy, 2014, 6:16; Moreth et al., Passive anti-amyloid immunotherapy in Alzheimer's disease: What are the most promising targets?, Immunity & Ageing, 2013, 10:18; www.alzforum.org), but currently no immunotherapy treatment has proven effective in clinical trials. In previous analysis of the murine 5E3 antibody, in vitro and in vivo studies indicated that neutralization of Aβ oligomer toxicity with murine 5E3 occurs independent of effector activation, including microglia and brain monocytes/macrophages. These results suggest that treatment with 5E3 antibody may mitigate the adverse effects, such as cerebral edema, hemorrhage or adverse inflammatory responses, seen with previous immune therapy against Aβ.

It is desirable to develop biologics that arrest or slow down the progression of Alzheimer's disease without inducing negative and potentially lethal effects on the human body. The need is particularly evident in view of the increasing longevity of the general population and, with this increase, an associated rise in the number of patients annually diagnosed with Alzheimer's disease. It is also desirable to develop diagnostic tools for determining the various stages of disease progression or for clinical stratification. Furthermore, monitoring the levels of Aβ and/or anti-Aβ-oligomer antibodies (or antigen-binding fragment thereof) during treatment can also be considered relevant within the current scope of the invention.

### SUMMARY

This disclosure provides for an isolated binding molecule, e.g., an antibody, or antigen-binding fragment thereof comprising a humanized antibody heavy chain variable domain (VH) and a humanized antibody light chain variable domain (VL). The binding molecule or fragment thereof has a VH less than 100% identical to SEQ ID NO: 16 that comprises the amino acid structure HFW1-HCDR1-HFW2-HCDR2-HFW3-HCDR3-HFW4, wherein HFW1 is SEQ ID NO: 22, or SEQ ID NO: 22 with one, two, three, four, or five single amino acid substitutions; HCDR1 is SEQ ID NO: 17, or SEQ ID NO: 17 with one, two, or three single amino acid substitutions; HFW2 is SEQ ID NO: 26, or SEQ ID NO: 26 with one, two, three, four, or five single amino acid substitutions; HCDR2 is SEQ ID NO: 18, or SEQ ID NO: 18 with one, two, or three, single amino acid substitutions; HFW3 is SEQ ID NO: 45, or SEQ ID NO: 45 with one, two, three, four, or five single amino acid substitutions; HCDR3 is SEQ ID NO: 19, or SEQ ID NO: 19 with one, two, or three single amino acid substitutions; and HFW4 is SEQ ID NO: 48, or SEQ ID NO: 48 with one, two, or three single amino acid substitutions. The binding molecule or fragment thereof also has a VL less than 100% identical to SEQ ID NO: 11 that comprises the amino acid structure LFW1-LCDR1-LFW2-LCDR2-LFW3-LCDR3-LFW4, wherein LFW1 is SEQ ID NO: 50, or SEQ ID NO: 50 with one, two, three, four, or five single amino acid substitutions; LCDR1 is SEQ ID NO: 12, or SEQ ID NO: 12 with one, two, or three single amino acid substitutions; LFW2 is SEQ ID NO: 52, or SEQ ID NO: 52 with one, two, three, four, or five single amino acid substitutions; LCDR2 is SEQ ID NO: 13, or SEQ ID NO: 13 with one single amino acid substitution; LFW3 is SEQ ID NO: 55, or SEQ ID NO: 55 with one, two, three, four, or five single amino acid substitutions; LCDR3 is SEQ ID NO: 14, or SEQ ID NO: 14 with one, two, or three single amino acid substitutions; and LFW4 is SEQ ID NO: 58, or SEQ ID NO: 58 with one, two, or three single amino acid substitutions. Further, the binding molecule or fragment thereof can bind to a cyclic peptide comprising the amino acid sequence SNK, wherein the K (Lysine) is solvent-accessible.

In certain aspects, the disclosure is directed to a binding molecule or fragment thereof as described herein that is an antibody or antigen-binding fragment thereof.

In certain embodiments, the HFW1 region of the binding molecule, e.g., antibody, or fragment thereof is SEQ ID NO: 22, SEQ ID NO: 23, or SEQ ID NO: 24. In certain embodiments, the HCDR1 region of the binding molecule, e.g., antibody, or fragment thereof is SEQ ID NO: 17, SEQ ID NO: 21, SEQ ID NO: 190 or SEQ ID NO: 192. In certain embodiments, the HFW2 region of the binding molecule, e.g., antibody, or fragment thereof is SEQ ID NO: 26, SEQ ID NO: 27, SEQ ID NO: 29, SEQ ID NO: 31, SEQ ID NO: 33, SEQ ID NO: 35, SEQ ID NO: 37, SEQ ID NO: 38, SEQ ID NO: 39, SEQ ID NO: 40, SEQ ID NO: 41, SEQ ID NO: 42, SEQ ID NO: 43, or SEQ ID NO: 44. In certain embodiments, the HCDR2 region of the binding molecule, e.g, antibody, or fragment thereof is SEQ ID NO: 18, SEQ ID NO: 194, SEQ ID NO: 196, SEQ ID NO: 198, SEQ ID NO: 200, SEQ ID NO: 202, or SEQ ID NO: 204. In certain embodiments, the HFW3 region of the binding molecule, e.g., antibody, or fragment thereof is SEQ ID NO: 45, SEQ ID NO: 46, or SEQ ID NO: 47. In certain embodiments, the HCDR3 region of the binding molecule, e.g., antibody, or fragment thereof is SEQ ID NO: 19, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, or SEQ ID NO: 206. In certain embodiments, the HFW4 region of the binding molecule, e.g., antibody, or fragment thereof is SEQ ID NO: 48 or SEQ ID NO: 49. In certain embodiments, the LFW1 region of the binding molecule, e.g., antibody, or fragment thereof is SEQ ID NO: 50 or SEQ ID NO: 51. In certain embodiments, the LCDR1 region of the binding molecule, e.g., antibody or fragment thereof is SEQ ID NO: 12, SEQ ID NO: 164, SEQ ID NO: 166, SEQ ID NO: 168, SEQ ID NO: 170, or SEQ ID NO: 172. In certain embodiments, the LFW2 region of the binding molecule, e.g., antibody, or fragment thereof is SEQ ID NO: 52, SEQ ID NO: 53, or SEQ ID NO: 54. In certain embodiments, the LCDR2 region of the binding molecule, e.g., antibody, or fragment thereof is SEQ ID NO: 13, SEQ ID NO: 174, SEQ ID NO: 176, SEQ ID NO: 178, SEQ ID NO: 180, SEQ ID NO: 182, or SEQ ID NO: 184. In certain embodiments, the LFW3 region of the binding molecule, e.g., antibody, or fragment thereof is SEQ ID NO: 55, SEQ ID NO: 56, or SEQ ID NO: 57. In certain embodiments, the LCDR3 region of the binding molecule, e.g., antibody, or fragment thereof is SEQ ID NO: 14, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 186, or SEQ ID NO: 188. In certain embodiments, the LFW4 region of the binding molecule, e.g., antibody, or fragment thereof is SEQ ID NO: 58 or SEQ ID NO: 59.

In certain embodiments, a VH that is less than 100% identical to SEQ ID NO: 16 can comprise the amino acid sequence SEQ ID NO: 63, SEQ ID NO: 67, SEQ ID NO: 71, SEQ ID NO: 124, SEQ ID NO: 126, SEQ ID NO: 128, SEQ ID NO: 130, SEQ ID NO: 132, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 217, SEQ ID NO: 218, SEQ ID NO: 219, SEQ ID NO: 220, SEQ ID NO: 221, SEQ ID NO: 136, SEQ ID NO: 136, SEQ ID NO: 138, SEQ ID NO: 140, and SEQ ID NO: 142. In certain embodiments, a VL that is less than 100% identical to SEQ ID NO: 11 can comprise the amino acid sequence SEQ ID NO: 61, SEQ ID NO: 65, or SEQ ID NO: 69. Thus, in certain embodiments, the VH and VL comprise, respectively, the amino acid sequences SEQ ID NO: 63 and SEQ ID NO: 61, SEQ ID NO: 67 and SEQ ID NO: 65, SEQ ID NO: 71 and SEQ ID NO: 69, SEQ ID NO: 124 and SEQ ID NO: 65, SEQ ID NO: 126 and SEQ ID NO: 65, SEQ ID NO: 128 and SEQ ID NO: 65, SEQ ID NO: 130 and SEQ ID NO: 65, SEQ ID NO: 132 and SEQ ID NO: 65, SEQ ID NO: 144 and SEQ ID NO: 65, SEQ ID NO: 146 and SEQ ID NO: 65, SEQ ID NO: 148 and SEQ ID NO: 65, SEQ ID NO: 150 and SEQ ID NO: 65, SEQ ID NO: 152 and SEQ ID NO: 65, SEQ ID NO: 154 and SEQ ID NO: 65, SEQ ID NO: 156 and SEQ ID NO: 65, SEQ ID NO: 158 and SEQ ID NO: 65, SEQ ID NO: 160 and SEQ ID NO: 65, SEQ ID NO: 162 and SEQ ID NO: 65, SEQ ID NO: 217 and SEQ ID NO: 65, SEQ ID NO: 218 and SEQ ID NO: 65, SEQ ID NO: 219 and SEQ ID NO: 65, SEQ ID NO: 220 and SEQ ID NO: 65, SEQ ID NO: 221 and SEQ ID NO: 65; SEQ ID NO: 144 and SEQ ID NO: 61, SEQ ID NO: 146 and SEQ ID NO: 61, SEQ ID NO: 148 and SEQ ID NO: 61, SEQ ID NO: 150 and SEQ ID NO: 61, SEQ ID NO: 152 and SEQ ID NO: 61, SEQ ID NO: 154 and SEQ ID NO: 69, SEQ ID NO: 156 and SEQ ID NO: 69, SEQ ID NO: 158 and SEQ ID NO: 69, SEQ ID NO: 160 and SEQ ID NO: 69, or SEQ ID NO: 162 and SEQ ID NO: 69.

In certain aspects, the binding molecule, e.g., antibody, or fragment thereof described herein can further comprise a light chain constant region or fragment thereof fused to the C-terminus of the VL such as, for example, wherein the light chain constant region is a human kappa constant region. The binding molecule, e.g., antibody, or fragment thereof described herein can further comprise a heavy chain constant region or fragment thereof fused to the C-terminus of the VH such as, for example, wherein the heavy chain constant region is a human IgG constant region or a human IgA constant region. In certain embodiments, the heavy chain constant region is a human IgG1 constant region, a human IgG2 constant region, or a human IgG4 constant region. In certain embodiments of a binding molecule, e.g., antibody, or antigen-binding fragment thereof, the antigen-binding fragment is an Fv fragment, an Fab fragment, an F(ab′)2 fragment, an Fab′ fragment, a dsFv fragment, an scFv fragment, or an sc(Fv)2 fragment, or any combination thereof.

Certain embodiments provide for a binding molecule, e.g., antibody, or fragment thereof which exhibits enhanced expression in transiently transfected CHO cells as compared to a corresponding antibody or fragment thereof comprising the VH amino acid sequence SEQ ID NO: 67 and the VL amino acid sequence SEQ ID NO: 65. In certain embodiments, the VL comprises the amino acid sequence SEQ ID NO: 65 and the VH comprises the amino acid sequence SEQ ID NO: 124, SEQ ID NO: 126, SEQ ID NO: 128, SEQ ID NO: 130, SEQ ID NO: 132, SEQ ID NO: 217, SEQ ID NO: 218, SEQ ID NO: 219, SEQ ID NO: 220, or SEQ ID NO: 221 and/or the VH and VL comprise, respectively, the amino acid sequences SEQ ID NO: 124 and SEQ ID NO: 65, SEQ ID NO: 126 and SEQ ID NO: 65, SEQ ID NO: 128 and SEQ ID NO: 65, SEQ ID NO: 130 and SEQ ID NO: 65, or SEQ ID NO: 132 and SEQ ID NO: 65.

The binding molecule, e.g., antibody, or fragment can bind to a cyclic peptide comprising the amino acid sequence SNK, wherein the K (Lysine) is solvent-accessible. The cyclic peptide can consists of 3, 4, 5, 6, 7, 8 or 9 amino acids such as, for example, a cyclic peptide comprising the amino acid sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9.

In certain embodiments, a binding molecule, e.g., antibody, or fragment thereof described herein has a dissociation constant (K_{D}) that is less than about 1×10⁻⁸ M. In certain embodiments, the binding molecule, e.g, antibody, or fragment thereof described herein can bind to an oligomeric form of Aβ at a greater affinity than to a non-oligomeric form of Aβ.

Certain aspects are drawn to a composition comprising the binding molecule or fragment thereof or the binding molecule, e.g., antibody, or fragment thereof described herein, and a pharmaceutically acceptable carrier. Other aspects are drawn to an isolated polynucleotide comprising a nucleic acid that encodes the binding molecule or fragment thereof or the biding molecule, e.g., antibody, or fragment thereof described herein, or a polypeptide subunit thereof.

Certain aspects are drawn to a vector comprising an isolated polynucleotide comprising a nucleic acid that encodes the binding molecule or fragment thereof or the antibody or fragment thereof described herein, or a polypeptide subunit thereof, and/or a cell comprising such polynucleotide and/or vector. In certain embodiments, the cell is a bacterial cell. In certain embodiments, the cell is a eukaryotic cell. In certain embodiments, the cell is a mammalian cell. In certain embodiments, the cell is COS-1, COS-7, HEK293, BHK21, CHO, BSC-1, Hep G2, SP2/0, HeLa, myeloma or lymphoma cells.

Certain aspects are drawn to a method of preventing and/or treating an Aβ associated disease comprising administering to a subject an effective amount of a binding molecule, e.g., antibody, or fragment thereof described herein. For example, certain embodiments provide for a method of preventing and/or treating Alzheimer's disease comprising administering to a subject an effective amount of a binding molecule, e.g., antibody, or fragment thereof described herein. In certain embodiments, the binding molecule, e.g., antibody, or fragment thereof is administered intravenously, subcutaneously, intramuscularly, intrathecally, transdermally, or orally. In certain embodiments, the method further comprises the step of administering to the subject a second agent.

### BRIEF DESCRIPTION OF THE DRAWINGS/FIGURES

FIG. 1A shows a qualitative assessment of the interactions between murine 5E3, cdr5E3, hu5E3 and rehu5E3 constructs and the cSNK:BSA conjugate using biolayer interferometry. Key to the sensor traces: A4, D4, and E4: three clonal isolates of murine 5E3; B4: Empty; C4: Irrelevant Ab; F4: cdr5E3 (IgG1); G4: hu5E3 (IgG1); and H4: rehu5E3 (IgG1).

FIG. 1B shows qualitative assessment of the interactions between murine 5E3, cdr5E3, hu5E3 and rehu5E3 constructs and the cSNK:BSA conjugate using biolayer interferometry. Key to the sensor traces: A4 D4, and E4: three clonal isolates of murine 5E3; B4: Empty; C4: murine IgG2b isotype control; F4: hu5E3 (IgG1); G4: hu5E3 (IgG2); H4: rehu5E3 (IgG2).

FIG. 1C shows a qualitative assessment of the interactions between murine 5E3, chimeric 5E3, cdr5E3, hu5E3 and rehu5E3 constructs and the cSNK:BSA conjugate using biolayer interferometry. Key to the sensor traces: A12: m5E3; B12: Irrelevant mAb; C12: hu5E3 (IgG1); D12: chimeric 5E3 (IgG4); E12: cdr5E3 (IgG4); F12: hu5E3 (IgG4); G12: rehu5E3 (IgG4); H12: empty.

FIG. 2A is a graph showing the immunoreactivity of murine 5E3, chimeric 5E3 IgG1, and humanized 5E3 IgG1 constructs (hu5E3, rehu5E3, and cdr5E3) to captured cSNK by ELISA.

FIG. 2B is a graph showing the immunoreactivity of murine 5E3 and humanized 5E3 IgG2 constructs (hu5E3 and rehu5E3) to captured cSNK by ELISA.

FIG. 2C is a graph showing the immunoreactivity of murine 5E3, chimeric 5E3 IgG4, and humanized 5E3 IgG4 constructs (hu5E3, rehu5E3, and cdr5E3) to captured cSNK by ELISA.

FIGS. 3A-D show the results of Western immunoblots with murine and humanized 5E3 constructs purified from CHOK1SV cells. Blots were probed with either murine 5E3 (m5E3), or humanized 5E3 IgG1, IgG2 and IgG4 mAbs against the cSNK epitope conjugated to BSA (BSA-cSNK). These blots were only positive for the cSNK epitope and did not bind the negative controls (unconjugated BSA or Ovalbumin).

FIG. 4A shows a Western immunoblot analysis of non-reduced 5 ug of BSA, cSNK conjugated to BSA (BSA-cSNK) and ovalbumin control (OVA) probed with purified hu5E3 IgG1.

FIG. 4B show a Western immunoblot analysis of non-reduced purified hu5E3 IgG1 probed with anti-human IgG.

FIGS. 5A-D show the expression analysis of Δ5E3 constructs by Western blot analysis. Sample mAbs from transient transfection of CHO were normalized by equivalent viable cell concentrations, separated by non-reducing SDS-PAGE and Western blotted to nitrocellulose. Thereafter, expression levels were determined by probing with goat anti-human IgG-HRP for detection. FIG. 5A shows expression levels from days 3, 5 and 7 post transient transfection of CHO comparing humanized 5E3 IgG1 variant (hu5E3-IgG1) to a high expressing positive control humanized IgG1 (huIgG1-positive control). This analysis shows day 3 expression levels are readily detectable but low in hu5E3-IgG1 variant. FIG. 5B shows enhanced expression in humanized Δ5E3 IgG1 variants (huΔ5E3-IgG1; KHA, IQA, IHR, KQA, IQR and KQR) constructs in comparison to hu5E3-IgG1. FIG. 5C shows enhanced expression in huΔ5E3 IgG1 variants (KHA, IHR, KQA, IQR and KQR) constructs in comparison to hu5E3-IgG1 and humanized IgG1 positive control. FIG. 5D shows enhanced expression in humanized Δ5E3 IgG1, IgG2 and IgG4 variants (huΔ5E3-IgG1, huΔ5E3-IgG2 and huΔ5E3-IgG4, respectively) in comparison to the hu5E3-IgG1, hu5E3-IgG2, hu5E3-IgG4 and humanized IgG1 positive control. M, prestained protein marker with corresponding molecular weight (kDa); Bl, blank lane.

FIG. 6 shows expression titer from transient expression in CHO cells. m5E3 represents the transient expression of the murine variant.

FIG. 7A is a graph showing the immunoreactivity of humanized 5E3 IgG1 and Δ5E3 IgG1 constructs to captured cSNK (CGSNKGG; head-to-tail cyclized peptide of SEQ ID NO: 3) by ELISA.

FIG. 7B is a graph showing the immunoreactivity of humanized 5E3 IgG1 and Δ5E3 IgG1 constructs to captured ccSNK (CCGSNKGC; head-to-tail cyclized peptide of SEQ ID NO: 8) by ELISA.

FIG. 8 shows an alignment of heavy chain variable regions of hu5E3-IgG1 and huΔ5E3-IgG1 (KQR variant in framework 2) variant.

FIG. 9A is a graph showing binding of hu5E3-IgG1 to Amyloid beta oligomers (Aβ oligomers) prepared from recombinant Aβ peptides by biacore.

FIG. 9B is a graph showing binding of hu5E3 IgG1, IgG2, and IgG4 isotypes to Amyloid beta oligomers (Aβ oligomers) prepared from recombinant Aβ peptides by biacore.

### DETAILED DESCRIPTION

It is to be noted that the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects or aspects of the disclosure, which can be had by reference to the specification as a whole. Accordingly, the terms defined immediately below are more fully defined by reference to the specification in its entirety.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring.”

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids are included within the definition of “polypeptide,” and the term “polypeptide” can be used instead of, or interchangeably with any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology, but is not necessarily translated from a designated nucleic acid sequence. It can be generated in any manner, including by chemical synthesis.

A polypeptide as disclosed herein can be of a size of about 3 or more, 5 or more, 7 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “non-naturally occurring” polypeptide, or any grammatical variants thereof, is a conditional term that explicitly excludes, but only excludes, those forms of the polypeptide that are well-understood by persons of ordinary skill in the art as being “naturally-occurring,” or that are, or might be at any time, determined or interpreted by a judge or an administrative or judicial body to be, “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain aspects, variants can be non-naturally occurring. Non-naturally occurring variants can be produced using art-known mutagenesis techniques. Variant polypeptides can comprise conservative or non-conservative amino acid substitutions, deletions or additions. Derivatives are polypeptides that have been altered so as to exhibit additional features not found on the original polypeptide. Examples include fusion proteins. Variant polypeptides can also be referred to herein as “polypeptide analogs.” As used herein a “derivative” of a polypeptide can also refer to a subject polypeptide having one or more amino acids chemically derivatized by reaction of a functional side group. Also included as “derivatives” are those peptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and ornithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides and antibodies of the present disclosure do not abrogate the binding of the polypeptide or antibody containing the amino acid sequence, to the antigen to which the binding molecule binds. Methods of identifying nucleotide and amino acid conservative substitutions which do not eliminate antigen-binding are well-known in the art (see, e.g., Brummell et al., Biochem. 32: 1180-1 187 (1993); Kobayashi et al., Protein Eng. 12(10):879-884 (1999); and Burks et al., Proc. Natl. Acad. Sci. USA 94: 412-417 (1997)).

The term “polynucleotide” is intended to encompass a singular nucleic acid as well as plural nucleic acids, and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, a “non-naturally occurring” polynucleotide, or any grammatical variants thereof, is a conditional definition that explicitly excludes, but only excludes, those forms of the polynucleotide that are well-understood by persons of ordinary skill in the art as being “naturally-occurring,” or that are, or that might be at any time, determined or interpreted by a judge or an administrative or judicial body to be, “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions which function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit β-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse β-glucuronidase.

As used herein, the term “sequence identity” refers to a relationship between two or more polynucleotide sequences or between two or more polypeptide sequences. When a position in one sequence is occupied by the same nucleic acid base or amino acid residue in the corresponding position of the comparator sequence, the sequences are said to be “identical” at that position. The percentage “sequence identity” is calculated by determining the number of positions at which the identical nucleic acid base or amino acid residue occurs in both sequences to yield the number of “identical” positions. The number of “identical” positions is then divided by the total number of positions in the comparison window and multiplied by 100 to yield the percentage of “sequence identity.” Percentage of “sequence identity” is determined by comparing two optimally aligned sequences over a comparison window. The comparison window for nucleic acid sequences can be, for instance, at least 20, 30, 40, 50, 60, 70, 80, 90, 100, 110, 120, 130, 140, 150, 160, 170, 180, 190, 200, 300, 400, 500, 600, 700, 800, 900 or 1000 or more nucleic acids in length. The comparison window for polypeptide sequences can be, for instance, at least 20, 30, 40, 50, 60, 70, 80, 90, 100, 110, 120, 130, 140, 150, 160, 170, 180, 190, 200, 300 or more amino acids in length. In order to optimally align sequences for comparison, the portion of a polynucleotide or polypeptide sequence in the comparison window can comprise additions or deletions termed gaps while the reference sequence is kept constant. An optimal alignment is that alignment which, even with gaps, produces the greatest possible number of “identical” positions between the reference and comparator sequences. Percentage “sequence identity” between two sequences can be determined using the version of the program “BLAST 2 Sequences” which is available from the National Center for Biotechnology Information, which program incorporates the programs BLASTN (for nucleotide sequence comparison) or BLASTP (for polypeptide sequence comparison), which programs are based on the algorithm of Karlin and Altschul (Proc. Natl. Acad. Sci. USA 90(12):5873-5877, 1993). When utilizing “BLAST 2 Sequences,” default parameters can be used for word size (3), open gap penalty (11), extension gap penalty (1), gap dropoff (50), expect value (10) and any other required parameter including but not limited to matrix option. Two nucleotide or amino acid sequences are considered to have “substantially similar sequence identity” or “substantial sequence identity” if the two sequences have at least 80%, at least 85%, at least 90%, at least 95%, at least 96%, at least 97%, at least 98%, or at least 99% sequence identity relative to each other.

Disclosed herein are certain binding molecules, or antigen-binding fragments, variants, or derivatives thereof. Unless specifically referring to full-sized antibodies, the term “binding molecule” encompasses full-sized antibodies as well as antigen-binding subunits, fragments, variants, analogs, or derivatives of such antibodies, e.g., engineered antibody molecules or fragments that bind antigen in a manner similar to antibody molecules, but which use a different scaffold.

As used herein, the term “binding molecule” refers in its broadest sense to a molecule that specifically binds to a receptor, e.g., an epitope or an antigenic determinant. As described further herein, a binding molecule can comprise one of more “antigen binding domains” described herein. A non-limiting example of a binding molecule is an antibody or fragment thereof that retains antigen-specific binding.

As used herein, the terms “binding domain” or “antigen binding domain” refer to a region of a binding molecule that is necessary and sufficient to specifically bind to an epitope. For example, an “Fv,” e.g., a variable heavy chain (VH) and variable light chain (VL) of an antibody, either as two separate polypeptide subunits or as a single chain, is considered to be a “binding domain.” Other binding domains include, without limitation, the variable heavy chain (VHH) of an antibody derived from a camelid species, or six immunoglobulin complementarity determining regions (CDRs) expressed in a fibronectin scaffold. A “binding molecule” as described herein can include one, two, three, four, five, six, seven, eight, nine, ten, eleven, twelve or more “antigen binding domains.”

The terms “antibody” and “immunoglobulin” can be used interchangeably herein. An antibody (or a fragment, variant, or derivative thereof as disclosed herein) includes at least the variable domain of a heavy chain (for camelid species) or at least the variable domains of a heavy chain and a light chain. Basic immunoglobulin structures in vertebrate systems are relatively well understood. See, e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and optionally includes a J chain and/or a secretory component, or an IgM antibody that includes ten or twelve complete heavy chains and ten or twelve complete light chains and optionally includes a J chain.

As will be discussed in more detail below, the term “immunoglobulin” comprises various broad classes of polypeptides that can be distinguished biochemically. Those skilled in the art will appreciate that heavy chains are classified as gamma, mu, alpha, delta, or epsilon, (γ, μ, α, δ, ε) with some subclasses among them (e.g., γ1-γ4 or α1-α2)). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgA₁, IgA₂, etc. are well characterized and are known to confer functional specialization. Modified versions of each of these classes and isotypes are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure.

Both the light and heavy chains are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant domains of the light chain (CL) and the heavy chain (CH1, CH2 or CH3) confer biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the CH3 (or CH4 in the case of IgM) and CL domains actually comprise the carboxy-terminus of the heavy and light chain, respectively.

As indicated above, a variable region (i.e., the “binding domain”) allows the binding molecule to selectively recognize and specifically bind epitopes on antigens. That is, the VL domain and VH domain, or subset of the complementarity determining regions (CDRs), of a binding molecule, e.g., an antibody combine to form the variable region that defines a three dimensional antigen binding site. More specifically, the antigen binding site is defined by three CDRs on each of the VH and VL chains. Certain antibodies form larger structures. For example, IgA can form a molecule that includes two H2L2 units, a J chain, and a secretory component, all covalently connected via disulfide bonds, and IgM can form a pentameric or hexameric molecule that includes five or six H2L2 units and optionally a J chain covalently connected via disulfide bonds.

The terms “light chain variable region” (also referred to as “light chain variable domain”, “VL”, “LCVR” or in some cases light chain) and “heavy chain variable region” (also referred to as “heavy chain variable domain”, “VH”, “HCVR” or in some cases heavy chain) refer to the variable binding region from an antibody light and heavy chain, respectively. The variable binding regions are made up of discrete, well-defined sub-regions known as “complementarity determining regions” (CDRs) and “framework regions” (FRs). In some embodiments, the FRs are humanized. The term “CH” refers to an “immunoglobulin heavy chain constant region” or a “heavy chain constant region,” which is further divisible, depending on the antibody isotype into CH1, CH2, and CH3 (IgA, IgD, IgG), or CH1, CH2, CH3, and CH4 domains (IgE, IgM).

The six “complementarity determining regions” or “CDRs” present in an antibody antigen-binding domain are short, non-contiguous sequences of amino acids that are specifically positioned to form the binding domain as the antibody assumes its three dimensional configuration in an aqueous environment. The remainder of the amino acids in the binding domain, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest,” Kabat, E., et al., U.S. Department of Health and Human Services, (1983); and Chothia and Lesk, J. Mol. Biol., 196:901-917 (1987), which are incorporated herein by reference in their entireties). In some embodiments, an antibody, or antigen-binding fragment thereof, contains at least one heavy chain variable region and/or at least one light chain variable region. The heavy chain variable region (or light chain variable region) typically contains three CDRs and four framework regions (FRs), arranged from amino-terminus to carboxyl-terminus in the following order: FR1, CDR1, FR2, CDR2, FR3, CDR3, FR4.

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE A
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

CDRs can also be determined using IMGT® (the international ImMunoGeneTics information system®) numbering. H: heavy chain; K: kappa or L: light chain. Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

Binding molecules, e.g., antibodies or antigen-binding fragments, variants, or derivatives thereof include, but are not limited to, polyclonal, monoclonal, human, humanized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019. Immunoglobulin or antibody molecules encompassed by this disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

By “specifically binds,” it is generally meant that a binding molecule, e.g., an antibody or fragment, variant, or derivative thereof binds to an epitope via its antigen binding domain, and that the binding entails some complementarity between the antigen binding domain and the epitope. According to this definition, a binding molecule is said to “specifically bind” to an epitope when it binds to that epitope, via its antigen binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10⁻² sec⁻¹, 10⁻² sec⁻¹, 5×10⁻³ sec⁻¹, 10⁻³ sec⁻¹, 5×10⁻⁴ sec⁻¹, 10⁻⁴ sec⁻¹, 5×10⁻⁵ sec⁻¹, or 10⁻⁵ sec⁻¹ 5×10⁻⁶ sec⁻¹, 10⁻⁶ sec⁻¹, 5×10⁻⁷ sec⁻¹ or 10⁻⁷ sec⁻¹.

A binding molecule, e.g., an antibody or antigen-binding fragment, variant, or derivative disclosed herein can be said to bind a target antigen with an on rate (k(on)) of greater than or equal to 10³ M⁻¹ sec⁻¹, 5×10³M⁻¹ sec⁻¹, 10⁴ M⁻¹ sec⁻¹, 5×10⁴ M⁻¹ sec⁻¹, 10⁵ M⁻sec⁻¹, 5×10⁵ M⁻¹ sec⁻¹, 10⁶ M⁻¹ sec⁻¹, or 5×10⁶ M⁻¹ sec⁻¹ or 10⁷ M⁻¹ sec⁻¹.

A binding molecule, e.g., an antibody or fragment, variant, or derivative thereof is said to competitively inhibit binding of a reference antibody or antigen binding fragment to a given epitope if it preferentially binds to that epitope to the extent that it blocks, to some degree, binding of the reference antibody or antigen binding fragment to the epitope. Competitive inhibition can be determined by any method known in the art, for example, competition ELISA assays. A binding molecule can be said to competitively inhibit binding of the reference antibody or antigen binding fragment to a given epitope by at least 90%, at least 80%, at least 70%, at least 60%, or at least 50%.

As used herein, the term “affinity” refers to a measure of the strength of the binding of an individual epitope with one or more binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of binding domains and an antigen. See, e.g., Harlow at pages 29-34. Avidity is related to both the affinity of individual binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a between a bivalent monoclonal antibody with a receptor present at a high density on a cell surface would also be of high avidity.

Binding molecules or antigen-binding fragments, variants or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen; a measure of relatedness between two different antigenic substances. Thus, a binding molecule is cross reactive if it binds to an epitope other than the one that induced its formation. The cross reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or K_{D} no greater than 5×10⁻² M, 10⁻² M, 5×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

Antibody fragments including single-chain antibodies or other binding domains can exist alone or in combination with one or more of the following: hinge region, CH1, CH2, CH3, or CH4 domains, J chain, or secretory component. Also included are antigen-binding fragments that can include any combination of variable region(s) with one or more of a hinge region, CH1, CH2, CH3, or CH4 domains, a J chain, or a secretory component. Binding molecules, e.g., antibodies, or antigen-binding fragments thereof can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al.

An “immunoglobulin constant region” or “constant region” refers to a peptide or polypeptide sequence that corresponds to or is derived from part or all of one or more immunoglobulin constant region domains. In some embodiments, an immunoglobulin constant region corresponds to or is derived from part or all of one or more constant region domains, but optionally not all constant region domains of a source antibody. In certain embodiments, the constant region comprises IgG CH2 and CH3 domains, e.g., from an IgG1. In certain embodiments, a constant region does not comprise a CH1 domain. In certain embodiments, constant region domains making up the constant region are human. In some embodiments, constant region domains used in accordance with the invention lack or have minimal effector functions of antibody-dependent cell-mediated cytotoxicity (ADCC) and for complement activation and complement-dependent cytotoxicity (CDC), while retaining the ability to bind some Fc receptors (such as FcRn, the neonatal Fc receptor) and/or retaining a relatively long half-life in vivo. In other variations, a fusion protein of this disclosure includes constant regions that retain such effector function of one or both of ADCC and CDC. In certain embodiments, a binding domain (e.g., comprising an immunoglobulin heavy and light variable chain), antibody or fragment thereof of this disclosure is fused to a human IgG1 constant region, wherein the IgG1 constant region has one or more of the following amino acids mutated: leucine at position 234 (L234), leucine at position 235 (L235), glycine at position 237 (G237), glutamate at position 318 (E318), lysine at position 320 (K320), lysine at position 322 (K322), or any combination thereof (numbering according to EU). For example, any one or more of these amino acids can be changed to alanine. In a further embodiment, an IgG1 Fc domain has each of L234, L235, G237, E318, K320, and K322 (according to EU numbering) mutated to an alanine (i.e., L234A, L235A, G237A, E318A, K320A, and K322A, respectively), and optionally an N297A mutation as well (e.g., essentially eliminating glycosylation of the CH2 domain).

As used herein, the term “heavy chain subunit” includes amino acid sequences derived from an immunoglobulin heavy chain, a binding molecule, e.g., an antibody comprising a heavy chain subunit includes at least one of: a VH domain, a CH1 domain, a hinge (e.g., upper, middle, and/or lower hinge region) domain, a CH2 domain, a CH3 domain, a CH4 domain, or a variant or fragment thereof. For example, a binding molecule, e.g., an antibody or fragment, variant, or derivative thereof can include, in addition to a VH domain, a CH1 domain; CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain aspects a binding molecule, e.g., an antibody or fragment, variant, or derivative thereof can include, in addition to a VH domain, a CH3 domain and a CH4 domain; or a CH3 domain, a CH4 domain, and a J chain. Further, a binding molecule for use in the disclosure can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule.

The heavy chain subunits of a binding molecule, e.g., an antibody or fragment thereof, can include domains derived from different immunoglobulin molecules. For example, a heavy chain subunit of a polypeptide can include a CH1 domain derived from an IgG1 molecule and a hinge region derived from an IgG3 molecule. In another example, a heavy chain subunit can include a hinge region derived, in part, from an IgG1 molecule and, in part, from an IgG3 molecule. In another example, a heavy chain subunit can comprise a chimeric hinge derived, in part, from an IgG1 molecule and, in part, from an IgG4 molecule.

As used herein, the term “light chain subunit” includes amino acid sequences derived from an immunoglobulin light chain. The light chain subunit includes at least one of a VL or CL (e.g., Cκ or Cλ) domain.

Binding molecules, e.g., antibodies or antigen-binding fragments, variants, or derivatives thereof can be described or specified in terms of the epitope(s) or portion(s) of an antigen that they recognize or specifically bind. The portion of a target antigen that specifically interacts with the antigen binding domain of an antibody is an “epitope,” or an “antigenic determinant.” A target antigen can comprise a single epitope or at least two epitopes, and can include any number of epitopes, depending on the size, conformation, and type of antigen.

As previously indicated, the subunit structures and three dimensional configuration of the constant regions of the various immunoglobulin classes are well known. As used herein, the term “VH domain” includes the amino terminal variable domain of an immunoglobulin heavy chain and the term “CH1 domain” includes the first (most amino terminal) constant region domain of an immunoglobulin heavy chain. The CH1 domain is adjacent to the VH domain and is amino terminal to the hinge region of a typical immunoglobulin heavy chain molecule.

As used herein the term “CH2 domain” includes the portion of a heavy chain molecule that extends, e.g., from about amino acid 244 to amino acid 360 of an IgG antibody using conventional numbering schemes (amino acids 244 to 360, Kabat numbering system; and amino acids 231-340, EU numbering system; see Kabat E A et al. op. cit. The CH3 domain extends from the CH2 domain to the C-terminal of the IgG molecule and comprises approximately 108 amino acids. Certain immunoglobulin classes, e.g., IgM, further include a CH4 region.

As used herein, the term “hinge region” includes the portion of a heavy chain molecule that joins the CH1 domain to the CH2 domain. This hinge region comprises approximately 25 amino acids and is flexible, thus allowing the two N-terminal antigen binding regions to move independently.

As used herein the term “disulfide bond” includes the covalent bond formed between two sulfur atoms. The amino acid cysteine comprises a thiol group that can form a disulfide bond or bridge with a second thiol group. In certain IgG molecules, the CH1 and CL regions are linked by a disulfide bond and the two heavy chains are linked by two disulfide bonds at positions corresponding to 239 and 242 using the Kabat numbering system (position 226 or 229, EU numbering system).

As used herein, the term “chimeric antibody” refers to an antibody in which the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The terms “multispecific antibody, or “bispecific antibody” refer to an antibody that has binding domains for two or more different epitopes within a single antibody molecule. Other binding molecules in addition to the canonical antibody structure can be constructed with two binding specificities. Epitope binding by bispecific or multispecific antibodies can be simultaneous or sequential. Triomas and hybrid hybridomas are two examples of cell lines that can secrete bispecific antibodies. Bispecific antibodies can also be constructed by recombinant means. (Ströhlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which the variable domain in either the heavy and light chain or both is altered by at least partial replacement of one or more amino acids in either the CDR or framework regions. In certain aspects entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain aspects not all of the CDRs are replaced with the complete CDRs from the donor variable region and yet the antigen binding capacity of the donor can still be transferred to the recipient variable domains. Using the information provided in this disclosure and the methods set forth in, e.g., U.S. Pat. Nos. 5,585,089, 5,693,761, 5,693,762, and 6,180,370, it will be well within the competence of those skilled in the art to obtain a functional engineered or humanized antibody.

As used herein the term “engineered” includes manipulation of nucleic acid or polypeptide molecules by synthetic means (e.g. by recombinant techniques, in vitro peptide synthesis, by enzymatic or chemical coupling of peptides or some combination of these techniques).

The disclosure also provides humanized and chimeric antibodies or antigen-binding fragments thereof, wherein the antibody or antigen-binding fragment thereof is affinity maturated.

A humanized antibody as provided herein can be derived from an antibody from a non-human species (e.g., mouse) where the amino acid sequence mostly in the non-antigen binding regions (and/or the antigen-binding regions) has been altered so that the antibody more closely resembles a human antibody, and still retains the ability to bind the corresponding antigen/epitope.

Humanized antibodies can be generated by replacing the framework regions of the murine VH and/or VL with equivalent or corresponding sequences from human variable regions. Those methods can include isolating, manipulating, and expressing the nucleic acid sequences that encode all or part of variable regions from at least one of a heavy or light chain.

Non-human binding domains can be humanized using techniques known as CDR grafting (Jones et al., Nature 321:522 (1986)) and variants thereof, including “reshaping” (Verhoeyen, et al., 1988 Science 239:1534-1536; Riechmann, et al., 1988 Nature 332:323-337; Tempest, et al., Bio/Technol 1991 9:266-271), “hyperchimerization” (Queen, et al., 1989 Proc Natl Acad Sci USA 86:10029-10033; Co, et al., 1991 Proc Natl Acad Sci USA 88:2869-2873; Co, et al., 1992 J Immunol 148:1149-1154), and “veneering” (Mark, et al., “Derivation of therapeutically active humanized and veneered anti-CD18 antibodies. In: Metcalf B W, Dalton B J, eds. Cellular adhesion: molecular definition to therapeutic potential. New York: Plenum Press, 1994: 291-312). If derived from a non-human source, other regions of the antibody or immunoglobulin binding proteins and polypeptides, such as the hinge region and constant region domains, can also be humanized.

An antibody light or heavy chain variable region consists of a framework region interrupted by three hypervariable regions, referred to as complementarity determining regions (CDRs). In one embodiment, humanized antibodies are antibody molecules from non-human species having 1, 2, 3, 4, 5 or 6 (all) CDRs from the non-human species and framework regions from a human immunoglobulin molecule.

Humanized antibodies as provided herein can be produced by methods known in the art. For example, once non-human (e.g., murine) antibodies are obtained, variable regions can be sequenced, and the location of the CDRs and framework residues determined. Kabat, E. A., et al. (1991) Sequences of Proteins of Immunological Interest, Fifth Edition, U.S. Department of Health and Human Services, NIH Publication No. 91-3242. Chothia, C. et al. (1987) J. Mol. Biol., 196:901-917. The light and heavy chain variable regions can, optionally, be ligated to corresponding constant regions. CDR-grafted antibody molecules can be produced by CDR-grafting or CDR substitution. One, two, or all CDRs of an immunoglobulin chain can be replaced. For example, all of the CDRs of a particular antibody can be from at least a portion of a non-human animal (e.g., mouse, such as CDRs shown in Table 12) or only some of the CDRs can be replaced. It is only necessary to keep the CDRs required for binding of the antibody to a predetermined antigen (e.g., a conformational epitope of oligomeric Aβ).

Also encompassed by the invention are antibodies or antigen-binding portions thereof containing one, two, or all CDRs of a variable region as disclosed herein, with the other regions replaced by sequences from at least one different species including, but not limited to, human, rabbits, sheep, dogs, cats, cows, horses, goats, pigs, monkeys, apes, gorillas, chimpanzees, ducks, geese, chickens, amphibians, reptiles and other animals.

Antibodies or antigen-binding fragments thereof can also include variants, analogs, orthologs, homologs and derivatives of peptides, that exhibit a biological activity, e.g., binding of an antigen. They can contain one or more analogs of an amino acid (including, for example, non-naturally occurring amino acids, amino acids which only occur naturally in an unrelated biological system, modified amino acids from mammalian systems etc.), peptides with substituted linkages, as well as other modifications known in the art.

The disclosure also provides antibodies or antigen-binding fragments thereof in which specific amino acids have been substituted, deleted, silenced or added. Some embodiments include glycosylation variants of an antibody or antigen-binding portion thereof described herein. In some embodiments, these alternations do not have a substantial effect on the peptide's biological properties such as binding activity, but can improve half-life and/or bioavailability. In some embodiments, alterations do alter (e.g., increase or decrease) affinity of a binding molecule (e.g., antibody or binding portion thereof). For example, antibodies can have amino acid substitutions in the framework region, such as to improve binding to the antigen. In addition, amino acid substitutions in the framework region can increase the heavy:light chain interface stability leading to increased expression (Mason et al, Identifying bottlenecks in transient and stable production of recombinant monoclonal-antibody sequence variants in Chinese hamster ovary cells, 2012, Biotechnology Progress 28(3):846-66). In another example, a selected (in some cases small) number of acceptor framework residues can be replaced by the corresponding donor amino acids. The donor framework can be a mature or germline human antibody framework sequence or a consensus sequence. Guidance concerning how to make phenotypically silent amino acid substitutions is provided in Bowie et al., Science, 247: 1306-1310 (1990).

In some embodiments, an antibody, or antigen-binding fragment thereof, can be derivatized or linked to another functional molecule. For example, an antibody can be functionally linked (by chemical coupling, genetic fusion, noncovalent interaction, etc.) to one or more other molecular entities, such as another antibody, a detectable agent, a cytotoxic agent, a pharmaceutical agent, a protein or peptide that can mediate association with another molecule (such as a streptavidin core region or a polyhistidine tag), or facilitate uptake across blood brain barrier (e.g., fusion to cholera toxin A subunits), block or interact with receptors, amino acid linkers, signal sequences, immunogenic carriers, or ligands useful in protein purification, such as glutathione-S-transferase, histidine tag, or staphylococcal protein A. One type of derivatized protein is produced by crosslinking two or more proteins (of the same type or of different types). Suitable crosslinkers include those that are heterobifunctional, having two distinct reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company; Rockford, Ill. Useful detectable agents with which a protein can be derivatized (or labeled) include fluorescent compounds, various enzymes, prosthetic groups, luminescent materials, bioluminescent materials, and radioactive materials. Non-limiting, exemplary fluorescent detectable agents include fluorescein, fluorescein isothiocyanate, rhodamine, and, phycoerythrin. A protein or antibody can also be derivatized with detectable enzymes, such as alkaline phosphatase, horseradish peroxidase, beta-galactosidase, acetylcholinesterase, glucose oxidase and the like. A protein can also be derivatized with a prosthetic group (e.g., streptavidin/biotin and avidin/biotin).

Peptides can be a functionally active variant of antibodies or of antigen-binding fragments thereof disclosed herein, e.g., with less than about 30%, about 25%, about 20%, about 15%, about 10%, about 5% or about 1% of amino acid residues substituted or deleted while retaining essentially the same immunological properties including, but not limited to, binding to a conformational epitope of oligomeric Aβ. Although the degree of the immunological property/activity (e.g., binding affinity) could change or remain essentially the same.

In some embodiments, methods of affinity maturation can be used to improve the activity of the original antibody. The process of affinity maturation can involve altering one or more amino acid residues to increase the activity of the antibody for a target antigen, such as for a conformational epitope of oligomeric Aβ. Improvements on the activity of the antibody can include, but is not limited to, increased binding affinity to the target antigen, increased expression levels and/or stability compared to the original antibody. In some cases a humanized antibody binding domain is affinity matured to obtain binding domains with desired characteristics such as increase binding affinity. An antibody that has gone through this process can be referred to as an affinity matured antibody.

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly a portion of a polypeptide that is “carboxy-terminal” or “C-terminal” to another portion of a polypeptide is that portion that comes later in the sequential polypeptide chain. For example in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide which is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

Terms such as “treating” or “treatment” or “to treat” or “alleviating” or “to alleviate” refer to therapeutic measures that cure, slow down, lessen symptoms of, and/or halt or slow the progression of an existing diagnosed pathologic condition or disorder. Terms such as “prevent,” “prevention,” “avoid,” “deterrence” and the like refer to prophylactic or preventative measures that prevent the development of an undiagnosed targeted pathologic condition or disorder. Thus, “those in need of treatment” can include those already with the disorder; those prone to have the disorder; and those in whom the disorder is to be prevented.

A “pharmaceutically effective amount” or a “therapeutically effective amount (or dose)” or “effective amount (or dose)” of a molecule or composition is that amount that produces a statistically significant effect in amelioration of one or more symptoms of the disorder (e.g., Alzheimer's disease), such as a statistically significant reduction or inhibition in disease progression or a statistically significant improvement, e.g., in organ or memory function. When referring to a combination, a therapeutically effective dose refers to combined amounts of the active ingredients that result in the therapeutic effect, whether administered serially or simultaneously (e.g., in the same formulation or concurrently in separate formulations) by a single or different routes of administration.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, phrases such as “a subject that would benefit from therapy” and “an animal in need of treatment” includes subjects, such as mammalian subjects, that would benefit from administration of a binding molecule, such as an antibody, comprising one or more antigen binding domains. Such binding molecules, e.g., antibodies, can be used, e.g., for a diagnostic procedures and/or for treatment or prevention of a disease.

Amyloid Beta (Aβ) Binding Molecules

This disclosure provides, inter alia, compositions and methods for the prevention or treatment of an amyloid disease such as Alzheimer's disease. In some embodiments, compositions contain an antibody or antibodies (or antigen-binding fragments thereof) that are specific to a conformational epitope of oligomeric Aβ. The conformational epitope corresponds to a solvent-exposed, antibody accessible knuckle region of oligomeric Aβ. The conformational epitope can be part of a cyclic peptide having an amino acid sequence comprising at least SNK (i.e., serine-asparagine-lysine or Ser-Asn-Lys) in which the side chain of lysine (K) is constrained to be oriented into solvent. The cyclic peptide can consist of 3, 4, 5, 6, 7, 8, or 9 amino acids. Suitable cyclic peptides can include without limitation the amino acid sequences SEQ ID NOs: 1 to 9. The present compositions can be used for passive or active immunotherapy of an amyloid disease such as Alzheimer's disease or as a prophylactic in populations at risk of Alzheimer's disease or other types of amyloid diseases.

As used herein, 5E3 refers to the hybridoma clone or the monoclonal antibodies comprising the variable heavy and light chain amino acid sequences in SEQ ID NOs: 16 and 11, respectively, or generated by the corresponding hybridoma clone (PCT Publication WO2011/106885).

In various embodiments, the antibodies or antigen-binding fragments thereof specifically bind to an epitope that overlaps with an epitope bound by an antibody produced by clone 5E3 and/or competes for binding to a conformational epitope of oligomeric Aβ with an antibody produced by clone 5E3. However, the antibodies or antigen-binding fragments provided herein differ from 5E3 monoclonal antibodies comprising the variable heavy amino acid sequence in SEQ ID NO: 16 and light chain amino acid sequence in SEQ ID NO: 11 because they comprise a humanized antibody heavy chain variable domain (VH) and a humanized antibody light chain variable domain (VL) in which the VH is less than 100% identical to SEQ ID NO: 16 and the VL is less than 100% identical to SEQ ID NO: 11. In certain aspects, the VL is less than 100%, 99%, 98%, 97%, 96%, 95%, 90%, 85%, 80%, 75%, of 70% identical to SEQ ID NO: 11. In certain aspects, the VH is less than 100%, 99%, 98%, 97%, 96%, 95%, 90%, 85%, 80%, 75%, of 70% identical to SEQ ID NO: 16.

A sequence of a heavy chain variable domain, light chain variable domain, heavy chain framework region, heavy chain complementary determining region, light chain framework region, light chain complementary determining region, etc., can vary from a similar sequence by one or more single amino acid substitutions. A single amino acid substitution refers to where the identity of an amino acid at a particular position in a polypeptide is changed to a different amino acid. In certain embodiments, the substitution is a conserved substitution as discussed elsewhere herein.

A humanized antibody heavy chain variable domain of the disclosure can be characterized as having an amino acid structure comprising from N-terminal to C-terminal the regions: heavy chain framework region 1 (HFW1)-heavy chain complementary determining region 1 (HCDR1)-heavy chain framework region 2 (HFW2)-heavy chain complementary determining region 2 (HCDR2)-heavy chain framework region 3 (HFW3)-heavy chain complementary determining region 3 (HCDR3)-heavy chain framework region 4 (HFW4) or: HFW1-HCDR1-HFW2-HCDR2-HFW3-HCDR3-HFW4.

A humanized antibody light chain variable domain of the disclosure can be characterized as having an amino acid structure comprising from N-terminal to C-terminal the regions: light chain framework region 1 (LFW1)-light chain complementary determining region 1 (LCDR1)-light chain framework region 2 (LFW2)-light chain complementary determining region 2 (LCDR2)-light chain framework region 3 (LFW3)-light chain complementary determining region 3 (LCDR3)-light chain framework region 4 (LFW4) or: LFW1-LCDR1-LFW2-LCDR2-LFW3-LCDR3-LFW4.

In certain embodiments, a HCDR1 region is SEQ ID NO: 17, or SEQ ID NO: 17 with any of one to eight single amino acid substitutions, for example, one, two, or three single amino acid substitutions. In certain embodiments, a HCDR2 region is SEQ ID NO: 18, or SEQ ID NO: 18 with any of one to eight single amino acid substitutions, for example, one, two, or three single amino acid substitutions. In certain embodiments, a HCDR3 region is SEQ ID NO: 19, or SEQ ID NO: 19 with any of one to nine single amino acid substitutions, for example, one, two, or three single amino acid substitutions.

In certain embodiments, a HFW1 region is SEQ ID NO: 22, or SEQ ID NO: 22 with any of one to ten single amino acid substitutions, for example, one, two, three, four, or five single amino acid substitutions. In certain embodiments, a HFW2 region is SEQ ID NO: 26, or SEQ ID NO: 26 with any of one to ten single amino acid substitutions, for example, one, two, three, four, or five single amino acid substitutions. In certain embodiments, a HFW3 region is SEQ ID NO: 45, or SEQ ID NO: 45 with any of one to ten single amino acid substitutions, for example, one, two, three, four, or five single amino acid substitutions. In certain embodiments, a HFW4 region is SEQ ID NO: 48, or SEQ ID NO: 48 with any of one to ten single amino acid substitutions, for example, one, two, or three single amino acid substitutions.

In certain embodiments, a LCDR1 region is SEQ ID NO: 12, or SEQ ID NO: 12 with any of one to six single amino acid substitutions, for example, one, two, or three single amino acid substitutions. In certain embodiments, a LCDR2 region is SEQ ID NO: 13, or SEQ ID NO: 13 with any of one to three single amino acid substitutions, for example one, two, or three single amino acid substitutions. In certain embodiments, a LCDR3 region is SEQ ID NO: 14, or SEQ ID NO: 14 with any of one to nine single amino acid substitutions, for example, one, two, or three single amino acid substitutions.

In certain embodiments, a LFW1 region is SEQ ID NO: 50, or SEQ ID NO: 50 with any of one to ten single amino acid substitutions, for example, one, two, three, four, or five single amino acid substitutions. In certain embodiments, a LFW2 region is SEQ ID NO: 52, or SEQ ID NO: 52 with any of one to ten single amino acid substitutions, for example, one, two, three, four, or five single amino acid substitutions. In certain embodiments, a LFW3 region is SEQ ID NO: 55, or SEQ ID NO: 55 with any of one to ten single amino acid substitutions, for example, one, two, three, four, or five single amino acid substitutions. In certain embodiments, a LFW4 region is SEQ ID NO: 58, or SEQ ID NO: 58 with any of one to ten single amino acid substitutions, for example, one, two, or three single amino acid substitutions.

In certain embodiments, in an antibody or fragment thereof, the heavy chain framework regions (i.e., HFW1, HFW2, HFW3, and HFW4) are human derived heavy chain framework regions (“hu”) and the heavy chain CDRs (i.e., HCDR1, HCDR2, and HCDR3) are identical or very similar to the heavy chain murine CDRs of 5E3 mAb and the light chain framework regions (i.e., LFW1, LFW2, LFW3, and LFW4) are human derived light chain framework regions (“hu”) and the light chain CDRs (i.e., LCDR1, LCDR2, and LCDR3) are identical or very similar to the murine light chain CDRs of 5E3 mAb. In one embodiment, HFW1 is SEQ ID NO: 22, or SEQ ID NO: 22 with one, two, three, four, or five single amino acid substitutions; HCDR1 is SEQ ID NO: 17, or SEQ ID NO: 17 with one, two, or three single amino acid substitutions; HFW2 is SEQ ID NO: 26, or SEQ ID NO: 26 with one, two, three, four, or five single amino acid substitutions; HCDR2 is SEQ ID NO: 18, or SEQ ID NO: 18 with one, two, or three, single amino acid substitutions; HFW3 is SEQ ID NO: 45, or SEQ ID NO: 45 with one, two, three, four, or five single amino acid substitutions; HCDR3 is SEQ ID NO: 19, or SEQ ID NO: 19 with one, two, or three single amino acid substitutions; and HFW4 is SEQ ID NO: 48, or SEQ ID NO: 48 with one, two, or three single amino acid substitutions; and LFW1 is SEQ ID NO: 50, or SEQ ID NO: 50 with one, two, three, four, or five single amino acid substitutions; LCDR1 is SEQ ID NO: 12, or SEQ ID NO: 12 with one, two, or three single amino acid substitutions; LFW2 is SEQ ID NO: 52, or SEQ ID NO: 52 with one, two, three, four, or five single amino acid substitutions; LCDR2 is SEQ ID NO: 13, or SEQ ID NO: 13 with one single amino acid substitution; LFW3 is SEQ ID NO: 55, or SEQ ID NO: 55 with one, two, three, four, or five single amino acid substitutions; LCDR3 is SEQ ID NO: 14, or SEQ ID NO: 14 with one, two, or three single amino acid substitutions; and LFW4 is SEQ ID NO: 58, or SEQ ID NO: 58 with one, two, or three single amino acid substitutions.

The humanized VH and humanized VL differ (i.e., are less than 100% identical to SEQ ID NO: 16 and SEQ ID NO: 11, respectively) by having CDR regions that are identical or very similar to SEQ ID NO: 12 (LCDR1), SEQ ID NO: 13 (LCDR2), SEQ ID NO: 14 (LCDR3), SEQ ID NO: 17 (HCDR1), SEQ ID NO: 18 (HCDR2), and SEQ ID NO: 19 (HCDR3) but encompassing wider variation among the frameworks regions which can be identical to or a variation of SEQ ID NO: 50 (LFW1), SEQ ID NO: 52 (LFW2), SEQ ID NO: 55 (LFW3), SEQ ID NO: 58 (LFW4), SEQ ID NO: 22 (HFW1), SEQ ID NO: 26 (HFW2), SEQ ID NO: 45 (HFW3), and SEQ ID NO: 48 (HFW4).

In certain embodiments, a HFW1 of SEQ ID NO: 22, or SEQ ID NO: 22 with one, two, three, four, or five single amino acid substitutions is SEQ ID NO: 22, SEQ ID NO: 23, or SEQ ID NO: 24. In certain embodiments, a HFW2 of SEQ ID NO: 26, or SEQ ID NO: 26 with one, two, three, four, or five single amino acid substitutions is SEQ ID NO: 26, SEQ ID NO: 27, SEQ ID NO: 29, SEQ ID NO: 31, SEQ ID NO: 33, SEQ ID NO: 35, SEQ ID NO: 37, SEQ ID NO: 38, SEQ ID NO: 39, SEQ ID NO: 40, SEQ ID NO: 41, SEQ ID NO: 42, SEQ ID NO: 43, or SEQ ID NO: 44. In certain embodiments, a HFW3 of SEQ ID NO: 45, or SEQ ID NO: 45 with one, two, three, four, or five single amino acid substitutions is SEQ ID NO: 45, SEQ ID NO: 46, or SEQ ID NO: 47. In certain embodiment, a HFW4 of SEQ ID NO: 48, or SEQ ID NO: 48 with one, two, or three single amino acid substitutions is SEQ ID NO: 48 or SEQ ID NO: 49.

In certain embodiments, a HCDR1 of SEQ ID NO: 17, or SEQ ID NO: 17 with one, two, or three single amino acid substitutions is SEQ ID NO: 17, SEQ ID NO: 21, SEQ ID NO: 190, or SEQ ID NO: 192. In certain embodiments, a HCDR2 or SEQ ID NO: 18, or SEQ ID NO: 18 with one, two, or three, single amino acid substitutions is SEQ ID NO: 18, SEQ ID NO: 194, SEQ ID NO: 196, SEQ ID NO: 198, SEQ ID NO: 200, SEQ ID NO: 202, or SEQ ID NO: 204. In certain embodiments, a HCDR3 of SEQ ID NO: 19, or SEQ ID NO: 19 with one, two, or three single amino acid substitutions is SEQ ID NO: 19, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, or SEQ ID NO: 206.

In certain embodiments, a LFW1 of SEQ ID NO: 50, or SEQ ID NO: 50 with one, two, three, four, or five single amino acid substitutions is SEQ ID NO: 50 or SEQ ID NO: 51. In certain embodiments, a LFW2 of SEQ ID NO: 52, or SEQ ID NO: 52 with one, two, three, four, or five single amino acid substitutions is SEQ ID NO: 52, SEQ ID NO: 53, or SEQ ID NO: 54. In certain embodiments, a LFW3 of SEQ ID NO: 55, or SEQ ID NO: 55 with one, two, three, four, or five single amino acid substitutions is SEQ ID NO: 55, SEQ ID NO: 56, or SEQ ID NO: 57. In certain embodiments, a LFW4 of SEQ ID NO: 58, or SEQ ID NO: 58 with one, two, or three single amino acid substitutions is SEQ ID NO: 58 or SEQ ID NO: 59.

In certain embodiments, a LCDR1 of SEQ ID NO: 12, or SEQ ID NO: 12 with one, two, or three single amino acid substitutions is SEQ ID NO: 12, SEQ ID NO: 164, SEQ ID NO: 166, SEQ ID NO: 168, SEQ ID NO: 170, or SEQ ID NO: 172. In certain embodiment, a LCDR2 of SEQ ID NO: 13, or SEQ ID NO: 13 with one single amino acid substitution is SEQ ID NO: 13, SEQ ID NO: 174, SEQ ID NO: 176, SEQ ID NO: 178, SEQ ID NO: 180, SEQ ID NO: 182, or SEQ ID NO: 184. In certain embodiments, a LCDR3 of SEQ ID NO: 14, or SEQ ID NO: 14 with one, two, or three single amino acid substitutions is SEQ ID NO: 14, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 186, or SEQ ID NO: 188.

In one embodiment, an antibody or antigen-binding fragment thereof, can undergo affinity maturation in any of the CDRs, e.g., HCDR1, HCDR2, HCDR3, LCDR1, LCDR2, and/or LCDR3. Exemplary modifications of the CDRs of the 5E3 monoclonal antibody (i.e., SEQ ID NOs 12-14 and 17-19) are presented in Table 12.

Certain modifications to the LCDR and HCDR regions resulted in increased affinity to a cyclic peptide comprising the amino acid sequence SNK, wherein the K (lysine) is solvent-accessible, compared to a LCDR3 of SEQ ID NO: 14 and HCDR3 of SEQ ID NO: 19, respectively. These included, but are not limited to: SEQ ID NO: 84 (LCDR3 L89I), SEQ ID NO: 110 (HCDR3 E209Q), SEQ ID NO: 114 (HCDR3 A210G), SEQ ID NO: 122 (HCDR3 Y212F), SEQ ID NO: 41, SEQ ID NO: 42, SEQ ID NO: 43, and SEQ ID NO: 44.

In one embodiment, an antibody or antigen-binding fragment thereof, can also be modified in any of the humanized framework regions, e.g., HFW1, HFW2, HFW3, HFW4, LFW1, LFW2, LFW3, and/or LFW4. Exemplary modifications of the humanized framework regions (i.e., SEQ ID NOs 22, 26, 45, 48, 50, 52, 55, and 58) are presented in Table 12.

Certain modifications to the HFW2 region resulted in increased transient expression over the murine 5E3 antibody. In certain embodiments, certain modifications to the HFW2 region result in increased transient expression of an antibody or fragment thereof as compared to a corresponding antibody or fragment thereof comprising the VH amino acid sequence SEQ ID NO: 16 and the VL amino acid sequence SEQ ID NO: 11. These include, but are not limited to, SEQ ID NO: 29 (HFW2 IHR), SEQ ID NO: 31 (HFW2 KHA), SEQ ID NO: 33 (HFW2 KQR), SEQ ID NO: 35 (HFW2 IQR) and SEQ ID NO: 37 (HFW2 KQA). Further, in certain embodiments, certain modifications to the HFW2 region resulted in increased transient expression of an antibody or fragment thereof as compared to a corresponding antibody or fragment thereof comprising the VH amino acid sequence SEQ ID NO: 67 and the VL amino acid sequence SEQ ID NO: 65. These include, but are not limited to, SEQ ID NO: 29 (HFW2 IHR), SEQ ID NO: 31 (HFW2 KHA), SEQ ID NO: 33 (HFW2 KQR), SEQ ID NO: 35 (HFW2 IQR) and SEQ ID NO: 37 (HFW2 KQA).

In certain embodiments, an antibody or fragment thereof of the disclosure further comprises a light chain constant region or fragment thereof fused to the C-terminus of the VL, for example, in one embodiment, the light chain constant region is a human kappa constant region. Likewise, in certain embodiments, an antibody or fragment thereof of the disclosure further comprises a heavy chain constant region or fragment thereof fused to the C-terminus of the VH, for example, in certain embodiments, the heavy chain constant region is a human IgG constant region or a human IgA constant region. In certain embodiments, a human IgG constant region is a human IgG1 constant region, human IgG2 constant region, or a human IgG4 constant region. In certain embodiments, the antibody or fragment thereof is fragment such as, for example, an Fv fragment, an Fab fragment, an F(ab′)2 fragment, an Fab′ fragment, a dsFv fragment, an scFv fragment, or an sc(Fv)2 fragment, or any combination thereof.

In certain embodiments, a humanized VL comprises SEQ ID NO: 61, SEQ ID NO:

65, or SEQ ID NO: 69. In certain embodiments, a humanized VH comprises SEQ ID NO: 63, SEQ ID NO: 67, SEQ ID NO: 71, SEQ ID NO: 124, SEQ ID NO: 126, SEQ ID NO: 128, SEQ ID NO: 130, SEQ ID NO: 132, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 217, SEQ ID NO: 218, SEQ ID NO: 219, SEQ ID NO: 220, or SEQ ID NO: 221. Thus, in certain embodiments, an antibody or antibody fragment is one wherein VL comprises the amino acid sequence of SEQ ID NO: 61 and VH comprises any one of SEQ ID NO: 63, SEQ ID NO: 67, SEQ ID NO: 71, SEQ ID NO: 124, SEQ ID NO: 126, SEQ ID NO: 128, SEQ ID NO: 130, SEQ ID NO: 132, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 217, SEQ ID NO: 218, SEQ ID NO: 219, SEQ ID NO: 220, or SEQ ID NO: 221. In certain other embodiments, an antibody or antibody fragment is one wherein VL comprises the amino acid sequence of SEQ ID NO: 65 and VH comprises any one of SEQ ID NO: 63, SEQ ID NO: 67, SEQ ID NO: 71, SEQ ID NO: 124, SEQ ID NO: 126, SEQ ID NO: 128, SEQ ID NO: 130, SEQ ID NO: 132, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 217, SEQ ID NO: 218, SEQ ID NO: 219, SEQ ID NO: 220, or SEQ ID NO: 221. In certain other embodiments, an antibody or antibody fragment is one wherein VL comprises the amino acid sequence of SEQ ID NO: 69 and VH comprises any one of SEQ ID NO: 63, SEQ ID NO: 67, SEQ ID NO: 71, SEQ ID NO: 124, SEQ ID NO: 126, SEQ ID NO: 128, SEQ ID NO: 130, SEQ ID NO: 132, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 217, SEQ ID NO: 218, SEQ ID NO: 219, SEQ ID NO: 220, or SEQ ID NO: 221.

Certain modifications result in enhanced expression of an antibody or fragment thereof, such as when expressed in transiently transfected CHO cells, as compared to the baseline expression obtained by an antibody or antibody fragment thereof comprising the VH amino acid sequence SEQ ID NO: 16 and the VL amino acid sequence SEQ ID NO: 11 and/or compared to the baseline expression obtained by an antibody or antibody fragment thereof comprising the VH amino acid sequence SEQ ID NO: 67 and the VL amino acid sequence SEQ ID NO: 65. Thus, in certain embodiments, an antibody or fragment thereof exhibits enhanced expression as compared to a corresponding antibody or fragment thereof comprising the VH amino acid sequence SEQ ID NO: 16 and the VL amino acid sequence SEQ ID NO: 11 and/or comprising the VH amino acid sequence SEQ ID NO: 67 and the VL amino acid sequence SEQ ID NO: 65. In certain embodiments, an antibody or fragment thereof exhibits enhanced expression in transiently transfected CHO cells as compared to a corresponding antibody or fragment thereof comprising the VH amino acid sequence SEQ ID NO: 16 and the VL amino acid sequence SEQ ID NO: 11 and/or comprising the VH amino acid sequence SEQ ID NO: 67 and the VL amino acid sequence SEQ ID NO: 65. Exemplary modifications resulting in enhanced expression compared to a corresponding antibody or fragment thereof comprising the VH amino acid sequence SEQ ID NO: 16 and the VL amino acid sequence SEQ ID NO: 11 and/or comprising the VH amino acid sequence SEQ ID NO: 67 and the VL amino acid sequence SEQ ID NO: 65, for example when expressed in transiently transfected CHO cells, include an antibody or fragment thereof wherein the VL comprises the amino acid sequence SEQ ID NO: 65 but the VH comprises the amino acid sequence SEQ ID NO: 124, SEQ ID NO: 126, SEQ ID NO: 128, SEQ ID NO: 130, SEQ ID NO: 132, SEQ ID NO: 217, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162.

As described, an antibody or antigen-binding fragment thereof can bind to a cyclic peptide comprising the amino acid sequence SNK, wherein the K (Lysine) is solvent-accessible. In certain embodiments, the dissociation constant (K_{D}) of the antibody, or antigen-binding fragment thereof, is less than about 1×10⁻⁸M. In certain embodiments, the dissociation constant (K_{D}) of the antibody, or antigen-binding fragment thereof, is between about 4.63×10⁻¹⁰ and about 1.82×10⁻¹⁰, between about 1.18×10⁻⁰⁹ and about 6.39×10⁻⁰⁹, between about 8.14×10⁻⁰⁹ and about 1.28×10⁻⁰⁹, between about 6.33×10⁻¹⁰ and about 3.04×10⁻¹⁰ and/or between about 5.57×10⁻¹⁰ and about 3.94×10⁻¹⁰. In certain embodiments, the dissociation constant (K_{D}) of the antibody, or antigen-binding fragment thereof, is between about 1.86×10⁻⁰⁹ and about 6.05×10⁻⁰⁹, between about 1.73×10⁻⁰⁹ and about 9.97×10⁻⁰⁹, between about 1.58×10⁻⁰⁹ and about 8.25×10⁻⁰⁹, or between about 1.62×10⁻⁰⁹ and about 1.74×10⁻⁰⁸. In certain embodiments, the dissociation constant (K_{D}) of the antibody, or antigen-binding fragment thereof, is about 2.01×10⁻⁰⁹, 1.52×10⁻⁰⁹, 1.43×10⁻⁰⁹, 1.28×10⁻⁰⁹, 1.26×10⁻⁰⁹, 1.88×10⁻⁰⁹, 3.26×10⁻¹⁰, 2.26×10⁻¹⁰, 1.31×10⁻⁰⁹, or 1.04×10⁻⁰⁹. In certain embodiments, the antibody or antigenic-binding fragment thereof can bind to an oligomeric form of Aβ at a greater affinity than to a non-oligomeric form of Aβ.

Polynucleotides, Vectors, and Host Cells

The disclosure also provides an isolated polynucleotide comprising a nucleic acid encoding a binding molecule or antigen-binding fragment thereof or an antibody or antigen-binding fragment thereof, or subunit thereof (e.g., a heavy chain and/or a light chain) as disclosed herein that specifically binds to a conformational epitope of oligomeric Aβ. A polynucleotide can comprise, for example, a nucleic acid that encodes all or part of an antibody, for example, one or both chains of the antibody, or a fragment, derivative, mutant or variant thereof. A polynucleotide can comprise one or more additional sequences, for example, regulatory sequences, and/or be part of a larger polynucleotide, for example, a vector. A nucleic acid can be expressed in a cell to produce an antibody or antigen-binding fragment thereof.

For example, the disclosure provides for a nucleic acid encoding an antibody or antibody-binding fragment thereof wherein HFW1 is SEQ ID NO: 22, or SEQ ID NO: 22 with one, two, three, four, or five single amino acid substitutions; HCDR1 is SEQ ID NO: 17, or SEQ ID NO: 17 with one, two, or three single amino acid substitutions; HFW2 is SEQ ID NO: 26, or SEQ ID NO: 26 with one, two, three, four, or five single amino acid substitutions; HCDR2 is SEQ ID NO: 18, or SEQ ID NO: 18 with one, two, or three, single amino acid substitutions; HFW3 is SEQ ID NO: 45, or SEQ ID NO: 45 with one, two, three, four, or five single amino acid substitutions; HCDR3 is SEQ ID NO: 19, or SEQ ID NO: 19 with one, two, or three single amino acid substitutions; and HFW4 is SEQ ID NO: 48, or SEQ ID NO: 48 with one, two, or three single amino acid substitutions; and LFW1 is SEQ ID NO: 50, or SEQ ID NO: 50 with one, two, three, four, or five single amino acid substitutions; LCDR1 is SEQ ID NO: 12, or SEQ ID NO: 12 with one, two, or three single amino acid substitutions; LFW2 is SEQ ID NO: 52, or SEQ ID NO: 52 with one, two, three, four, or five single amino acid substitutions; LCDR2 is SEQ ID NO: 13, or SEQ ID NO: 13 with one single amino acid substitution; LFW3 is SEQ ID NO: 55, or SEQ ID NO: 55 with one, two, three, four, or five single amino acid substitutions; LCDR3 is SEQ ID NO: 14, or SEQ ID NO: 14 with one, two, or three single amino acid substitutions; and LFW4 is SEQ ID NO: 58, or SEQ ID NO: 58 with one, two, or three single amino acid substitutions.

Based upon the genetic code, one of ordinary skill in the art can determine the nucleic acid sequence of a nucleic acid encoding a particular amino acid sequence. Exemplary sequences of nucleic acids that encode for one of a VH, VL, HFW1, HCDR1, HFW2, HCDR2, HFW3, HCDR3, HFW4, LFW1, LCDR1, LFW2, LCDR2, LFW3, LCDR3, or LFW4, include but are not limited to SEQ ID NO: 20, SEQ ID NO: 25, SEQ ID NO: 28, SEQ ID NO: 30, SEQ ID NO: 32, SEQ ID NO: 34, SEQ ID NO: 36, SEQ ID NO: 60, SEQ ID NO: 62, SEQ ID NO: 64, SEQ ID NO: 66, SEQ ID NO: 68, SEQ ID NO: 70, SEQ ID NO: 72, SEQ ID NO: 73, SEQ ID NO: 74, SEQ ID NO: 75, SEQ ID NO: 76, SEQ ID NO: 77, SEQ ID NO: 78, SEQ ID NO: 79, SEQ ID NO: 80, SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, SEQ ID NO: 111, SEQ ID NO: 113, SEQ ID NO: 115, SEQ ID NO: 117, SEQ ID NO: 119, SEQ ID NO: 121, SEQ ID NO: 123, SEQ ID NO: 125, SEQ ID NO: 127, SEQ ID NO: 129, SEQ ID NO: 131, SEQ ID NO; 133, SEQ ID NO: 135, SEQ ID NO: 137, SEQ ID NO: 139, SEQ ID NO: 141, SEQ ID NO: 143, SEQ ID NO: 145, SEQ ID NO: 147, SEQ ID NO: 149, SEQ ID NO: 151, SEQ ID NO: 153, SEQ ID NO: 155, SEQ ID NO: 157, SEQ ID NO: 159, SEQ ID NO: 161, SEQ ID NO: 163, SEQ ID NO: 165, SEQ ID NO: 167, SEQ ID NO: 169, SEQ ID NO: 171, SEQ ID NO: 173, SEQ ID NO: 175, SEQ ID NO: 177, SEQ ID NO: 179, SEQ ID NO: 181, SEQ ID NO: 183, SEQ ID NO: 185, SEQ ID NO: 187, SEQ ID NO: 189, SEQ ID NO: 191, SEQ ID NO: 193, SEQ ID NO: 195, SEQ ID NO: 197, SEQ ID NO: 199, SEQ ID NO: 201, SEQ ID NO: 203, or SEQ ID NO: 205.

Recoding can also be used to change the chemical make-up of a DNA and/or an RNA coding sequence such as the guanine/cytosine (GC) percentage. Depending on the particular situation or expression vector it can advantageous to increase or decrease the GC percentage in the recoded sequence.

Recoding can also be used to remove or add particular motifs to a coding sequence or polynucleotide such as procarya inhibitory motifs, consensus splice donor sites, cryptic splice donor sites or a combination thereof. In some embodiments, a recoded coding sequence has less procarya inhibitory motifs, consensus splice donor sites, cryptic splice donor sites or any combination thereof than the native sequence. In some embodiments, a recoded coding sequence contains no procarya inhibitory motifs, consensus splice donor sites and/or cryptic splice donor sites.

Hoover et al. (Nucleic Acids Res. (2002) 30:e43, pp 1-7); U.S. Patent Application 20070141557; Fath et al. (PLoS ONE (2011) 6:e17596 pp 1-14); and Graf et al. (J Virol (2000) 74:10822-10826 describe some examples of recoding and/or codon optimizing coding regions.

The invention also includes expression vectors including polynucleotides disclosed herein.

In another aspect, the invention includes vectors comprising a nucleic acid encoding an antibody, or antigen-binding fragment thereof. The invention includes vectors comprising these nucleic acids and cells the nucleic acids and vectors.

Examples of vectors include, but are not limited to, plasmids, viral vectors, non-episomal mammalian vectors and expression vectors, for example, recombinant expression vectors.

A recombinant expression vector can comprise a nucleic acid in a form suitable for expression of the nucleic acid in a host cell. The recombinant expression vectors include one or more regulatory sequences, selected on the basis of the host cells to be used for expression, which is operably linked to the nucleic acid sequence to be expressed. The regulatory sequence can, for example, exert its effects directly on the regulated nucleic acid, or through the action of one or more other molecules (e.g., polypeptides that bind to the regulatory sequence and/or the nucleic acid). Examples of regulatory sequences include promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06. Regulatory sequences include those that direct constitutive expression of a nucleotide sequence in many types of host cells (e.g., SV40 early gene enhancer, Rous sarcoma virus promoter, cytomegalovirus promoter, etc.), those that direct expression of the nucleotide sequence only in certain host cells (e.g., tissue-specific regulatory sequences, see Voss et al., 1986, Trends Biochem. Sci. 11:287, Maniatis et al., 1987, Science 236:1237), and those that direct inducible expression of a nucleotide sequence in response to particular treatment or condition (e.g., the metallothionin promoter in mammalian cells, the tet-responsive and/or streptomycin responsive promoter in both prokaryotic and eukaryotic systems, etc.). It will be appreciated by those skilled in the art that the design of the expression vector can depend on such factors as the choice of the host cell to be transformed, the level of expression of protein desired, etc. The expression vectors can be introduced into host cells to thereby produce proteins or peptides, including fusion proteins or peptides, encoded by nucleic acids as described herein.

Changes can be introduced by mutation into a nucleic acid, thereby leading to changes in the amino acid sequence of a polypeptide (e.g., an antibody or fragment thereof) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property (e.g., binding to a conformational epitope of oligomeric Aβ).

Nucleic acid molecules encoding a functionally active variant of an antibody or antigen-binding fragment thereof are also encompassed by the present invention. In some embodiments, these nucleic acid molecules hybridize under medium stringency, high stringency, or very high stringency conditions with a nucleic acid encoding any of the antibodies or antigen-binding fragments thereof disclosed herein. Guidance for performing hybridization reactions can be found in Current Protocols in Molecular Biology, John Wiley & Sons, N.Y. 6.3.1-6.3.6, 1989, which is incorporated herein by reference. Specific hybridization conditions referred to herein are as follows: 1) medium stringency hybridization conditions: 6×SSC at about 45° C., followed by one or more washes in 0.2×SSC, 0.1% SDS at 60° C.; 2) high stringency hybridization conditions: 6×SSC at about 45° C., followed by one or more washes in 0.2×SSC, 0.1% SDS at 65° C.; and 3) very high stringency hybridization conditions: 0.5 M sodium phosphate, 7% SDS at 65° C., followed by one or more washes at 0.2×SSC, 1% SDS at 65° C.

In some embodiments, a nucleic acid encoding an antibody or antigen-binding fragment thereof can be introduced into an expression vector that can be expressed in a suitable expression system, and optionally followed by isolation or purification of the expressed antibody or antigen-binding fragment thereof. Optionally, a nucleic acid encoding an antibody or antigen-binding fragment thereof can be translated in a cell-free translation system. U.S. Pat. No. 4,816,567. Queen et al., Proc Natl Acad Sci USA, 86:10029-10033 (1989).

Antibodies or fragments thereof can be produced, e.g., by host cells transformed with DNA encoding light and heavy chains (or fragments thereof) of a desired antibody. Antibodies can be isolated and purified from these culture supernatants and/or cells using standard techniques. For example, a host cell can be transformed with DNA encoding the light chain, the heavy chain, or both, of an antibody or binding fragment thereof. Recombinant DNA technology can also be used to remove some or all of the DNA encoding either or both of the light and heavy chains that is not necessary for binding, e.g., the constant region.

Nucleic acids can be expressed in various suitable cells, including prokaryotic and eukaryotic cells, e.g., bacterial cells, (e.g., E. coli), yeast cells, plant cells, insect cells, and mammalian cells. The present invention also provides for cells comprising the nucleic acids described herein. The cells can be a hybridoma or transfectant. A number of mammalian cell lines are known in the art and include immortalized cell lines available from the American Type Culture Collection (ATCC). Non-limiting examples of the cells include all cell lines of mammalian origin or mammalian-like characteristics, including but not limited to, parental cells, derivatives and/or engineered variants of monkey kidney cells (e.g., COS, e.g., COS-1, COS-7), HEK293 cells, baby hamster kidney cells (e.g., BHK, e.g., BHK21), Chinese hamster ovary cells (e.g., CHO), NS0 cells, PerC6 cells, BSC-1 cells, human hepatocellular carcinoma cells (e.g., Hep G2), SP2/0 cells, HeLa cells, Madin-Darby bovine kidney (MDBK) cells, myeloma cells and lymphoma cells. Engineered variants can include, e.g., glycan profile modified and/or site-specific integration site derivatives.

Alternatively, an antibody or antigen-binding fragment thereof can be synthesized by solid phase procedures well known in the art. Solid Phase Peptide Synthesis: A Practical Approach by E. Atherton and R. C. Sheppard, published by IRL at Oxford University Press (1989). Methods in Molecular Biology, Vol. 35: Peptide Synthesis Protocols (ed. M. W. Pennington and B. M. Dunn), chapter 7. Solid Phase Peptide Synthesis, 2nd Ed., Pierce Chemical Co., Rockford, Ill. (1984). G. Barany and R. B. Merrifield, The Peptides: Analysis, Synthesis, Biology, editors E. Gross and J. Meienhofer, Vol. 1 and Vol. 2, Academic Press, New York, (1980), pp. 3-254. M. Bodansky, Principles of Peptide Synthesis, Springer-Verlag, Berlin (1984).

Immunoassays

Antibodies against a conformational epitope of oligomeric Aβ can be characterized for binding to the antigen by a variety of known techniques. For example, in an ELISA, microtiter plates are coated with the antigen in PBS, and then blocked with irrelevant proteins such as bovine serum albumin (BSA) diluted in PBS. Dilutions of plasma from antigen-immunized mice are added to each well and incubated. The plates are washed and then incubated with a secondary antibody conjugated to an enzyme (e.g., alkaline phosphatase). After washing, the plates are developed with the enzyme's substrate (e.g., ABTS), and analyzed at a specific OD. In other embodiments, to determine if the selected monoclonal antibodies bind to unique epitopes, the antibody can be biotinylated which can then be detected with a streptavidin labeled probe. Anti-antigen antibodies can be tested for reactivity with the antigen by Western blotting.

Antibodies can also be assayed by in vitro multiplex bead-based immunoassays. Multiplex bead-based immunoassays, such as the Luminex® xMAP® technology, allow the measurement of one analyte or simultaneous measurement of multiple analytes using a library of antigen-containing (or epitope-containing) peptides (or proteins) coupled to color-coded beads. Each bead is identified by the unique wavelength it emits when excited by a laser. Quantitation is accomplished by a sandwich assay using a fluorescently labeled detection antibody with affinity to the specific analyte captured by the bead-coupled antibody beads. Excitation by a second laser reads the quantity of bound detection antibody. Houser, Brett, Using Bead-Based Multiplexing Immunoassays to Explore Cellular Response to Drugs, Drug Discover and Development, May 9, 2011. The beads that can be used in the Luminex® xMAP® immunoassays include MagPlex®, MicroPlex®, LumAvidin®, SeroMAP™ microspheres, etc. MagPlex® microspheres are superparamagnetic microspheres which are internally labeled with fluorescent dyes and contain surface carboxyl groups for covalent attachment of ligands (or biomolecules). Baker et al., Conversion of a Capture ELISA to a Luminex® xMAP® Assay using a Multiplex Antibody Screening Method, J. Vis. Exp., (65), e4084 10.3791/4084, DOI: 10.3791/4084 (2012). Fulton et al., Advanced multiplexed analysis with the FlowMetrix system. Clinical Chemistry, 43, 1749-1756 (1997). Carson et al., Simultaneous quantitation of 15 cytokines using a multiplexed flow cytometric assay, J. Immunol. Methods, 227, 41-52 (1999).

In one embodiment, to detect the presence of antibodies to the conformational epitope of a cyclic peptide in a sample, the cyclic peptides, having an amino acid sequence of at least SNK, e.g., SEQ ID NOs 1-9, are coupled to MagPlex® microspheres based on the multiplexing xMAP® platform and analyzed on the MagPix® instrument (Luminex Corporation, Austin, Tex.). The sample is then contacted with cyclic peptide-coupled microspheres, an immunoassay is used to detect and quantify antibodies specific to the cyclic peptide.

Different peptides, e.g., Aβ (1-42), Aβ (1-40), Aβ-derivatives and cyclic peptides as disclosed herein, can be coupled to different sets or regions of MagPlex® microspheres. Because each of these regions has a unique internal fluorescent dye, the immunoassay is able to discriminate between the antibodies specific to the different peptides.

Biacore™ assay can be used to characterize antibodies by measuring protein-protein interaction and binding affinity based on surface plasmon resonance (SPR). Karlsson et al., Analysis of active antibody concentration, Journal of Immunological Methods, (1993) 166, 75-84. Markey F., Measuring concentration, Biajournal, 1999, 2: 8-11. Antibody titers can also be measured by radioimmunoassay.

Antibodies that bind to the conformational epitope of Aβ can then be subcloned and further characterized.

Neuronal toxicity assays can also be used to measure activity of antibodies or antigen-binding fragments thereof. For example, in vitro neuronal toxicity assays can be used to measure the ability of an antibody to inhibit the toxic effect oligomeric Aβ on cultured cells, and thus increase cell survival. In one embodiment, in an in vitro neuronal toxicity assay, an antibody, or antigen-binding fragment thereof, at a concentration ranging about 0.1 mg/ml to about 5 mg/ml, about 1 mg/ml to about 2 mg/ml, about 10 mg/ml to about 1 mg/ml, about 50 mg/ml to about 500 mg/ml, about 100 mg/ml to about 400 mg/ml, about 180 mg/ml to about 360 μg/ml, about 0.01 mg/ml to about 5 mg/ml, improves the survival rate of the cells compared to a control sample. The percentages of increase of cell survival caused by an antibody, or antigen-binding fragment thereof, compared to a control sample, can be greater than about 10%, greater than about 20%, greater than about 30%, greater than about 40%, greater than about 50%, greater than about 60%, greater than about 70%, greater than about 80%, greater than about 90%, greater than about 95%, or greater than about 99%.

Antibodies, or antigen-binding fragments, variants or derivatives thereof of the present disclosure can also be described or specified in terms of their binding affinity to an antigen. The affinity of an antibody for an antigen can be determined experimentally using any suitable method (see, e.g., Berzofsky et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular antibody-antigen interaction can vary if measured under different conditions (e.g., salt concentration, pH) or different matrixes (e.g. serum, tissue homogenates). Thus, measurements of affinity and other antigen-binding parameters (e.g., K_{D}, Kₐ and K_{d}) can be made with standardized solutions of antibody and antigen, and a standardized buffer.

In some embodiments, an antibody or antigen-binding fragment thereof can have a greater affinity to an oligomeric form of Aβ than to a non-oligomeric form of Aβ. In some embodiments, an antibody or antigen-binding fragment thereof binds to an oligomeric form of Aβ with a dissociation constant (K_{D}) ranging from about 2 times to about 10¹⁰ times, about 5 times to about 10⁸ times, about 10 times to about 10⁶ times, about 100 times to about 10⁴ times, about 2 times to about 50 times, about 4 times to about 40 times, greater than about 4 times, greater than about 6 times, greater than about 8 times, greater than about 10 times, greater than about 30 times smaller than the K_{D} for a non-oligomeric form of Aβ.

When Aβ oligomerizes, a constrained peptide turn forms and takes on a knuckle-like conformation. In the knuckle region of oligomeric Aβ, the epitope GSNKG, including the lysine side chain, is exposed to solvent and accessible to antibody binding. This epitope represents a novel target in misfolded forms of Aβ or oligomers of Aβ including soluble oligomers. As used herein, the term “Aβ oligomer”, “AβO” or “oligomeric Aβ” refers to a form of the Aβ peptide where the Aβ monomers are non-covalently or covalently aggregated.

Image capture of molecular dynamics modeling of a disulfide-linked cyclic peptide comprising residues 25-29 (CGSNKGC SEQ ID NO: 7) was conducted; non-native cysteines were added for disulfide linkage. This modeling reveals that the side chain of lysine 28 is oriented externally, in contrast to the internally oriented lysine 28 side chain predicted in references Luhrs et al., Proc. Natl. Acad. Sci. USA, 2005, 102(48): 17342-7 and Rauk, A., Dalton Trans., 2008(10): 1273-82. The discovery of the outward orientation of the lysine 28 residue is consistent with the high immunogenicity of this cyclic peptide comprising residues 25-29 (CGSNKGC SEQ ID NO: 7), the side chain of lysine being solvent exposed and charged via an ε-amino group. The discovery of the outward orientation of the lysine 28 residue is consistent with authentic Aβ oligomers also displaying a similar lysine side-chain orientation in solvent in an antibody-accessible fashion. The serine 26, asparagine 27 and lysine 28 residues, SNK, located in the knuckle region of Aβ oligomers are all charged or polar, and have greater immunogenicity than small non-polar amino acids. The cyclic conformation of the SNK residues, located in the knuckle region of Aβ oligomers, form a novel conformational epitope that is solvent exposed and available for antibody binding. This constrained epitope at the surface of Aβ oligomers has advantageous properties for selective antibody binding.

In one aspect, the epitope is comprised of strongly polar/charged residues that are solvent-exposed and structurally constrained at the surface of Aβ oligomers. In another aspect, the structure of the novel conformation-specific epitope is dependent on a relatively-rigid spatial arrangement of the amino acid residues.

In some embodiments, antibodies or antigen-binding fragments thereof can have a greater affinity to an oligomeric form of Aβ than to a non-oligomeric form of Aβ.

In one embodiment, a conformational epitope having a constrained cyclic configuration is not present on the molecular surface of APP (amyloid precursor protein) thus limiting the autoimmune recognition of APP. The GSNKG (SEQ ID NO: 2) motif of APP that is located at the cell surface of neurons and monocytes is largely unstructured/linear. Conformation-specific antibodies binding to the novel conformational epitope having a constrained cyclic configuration have limited or no recognition of the linear GSNKG (SEQ ID NO: 2) motif on cell surface APP. In some embodiments, antibodies recognizing the conformational epitope show little or no reaction with monomeric Aβ (monomeric).

In another embodiment, antibodies binding to the conformational epitope having a constrained cyclic configuration recognize the nonlinear epitope structure in between the subunits in the region of amino acids 25-29 of Aβ oligomers. The specificity of the antibodies to the novel conformational epitope enables the antibodies to specifically target the oligomeric form of Aβ and as such, avoid targeting monomeric Aβ and APP that are known to impact on neuronal and immune function and increase the availability of the antibody for binding as monomeric Aβ is present in much larger quantities than oligomeric A, e.g., see PCT Publication No. WO 2011/106885.

The disclosure also provides peptides derived from or designed to mimic the AP comprising conformational epitope. This conformational epitope mimics the knuckle-like epitope in the misfolded, oligomeric Aβ. In one embodiment, the conformational epitope has an amino acid sequence comprising SNK. In another embodiment, the conformational epitope can be part of a cyclic peptide having an amino acid sequence comprising at least SNK. The peptide can be a cyclic peptide. The peptide can be derived from Aβ.

In some embodiments, a binding domain, binding protein, antibody or antigen-binding fragment thereof binds a cyclic peptide comprising the amino acid sequence SNK, wherein the K (Lysine) is solvent-accessible and wherein the binding domain, binding protein, antibody or antigen-binding fragment binds an epitope comprised of SNK, at least one amino acid or at least two amino acids in the SNK sequence. In some embodiments, the cyclic peptide is selected from of SEQ ID NO: 2-9.

In some embodiments, an isolated humanized antibody, or an antigen-binding fragment thereof binds to the same epitope recognized by an antibody comprising a VH and a VL having the amino acid sequences SEQ ID NOs: 16 and 11, respectively. This can be determined, for example, by performing competition assays (e.g., using ELISA or BIACORE™ based assays), e.g., as described in Schalkhammer, Analytical Biotechnology (2002) published by Birkhäauser and Crowther, ELISA: Theory and Practice (1995) Humana Press or through X-ray crystallography.

The conformational epitope peptide can have 5, 6, 7, 8, 9, 10, 11, 12, 13, 14 or more amino acid residues. The conformational epitope peptide can have less than 6, less than 7, less than 8, less than 9, less than 10, less than 11, less than 12, less than 13, less than 14 or less than 15 amino acid residues.

In one embodiment, the ring of the cyclic peptide can contain 5, 6, 7, 8, 9 or 10 amino acid residues. The ring of the cyclic peptide can contain less than 6, less than 7, less than 8, less than 9, less than 10 or less than 11 amino acid residues.

A conformational epitope can be part of a disulfide-linked cyclic peptide. The cyclization of the peptide can also be through any other suitable covalent bonds. The conformational epitope can be naturally occurring. A peptide can be cyclic, non-cyclic, branched, linear, or any other suitable form that can give a constrained configuration corresponding to the conformational epitope in oligomeric Aβ. This conformational epitope is described in detail in PCT Publication No. WO2011/106885.

As used herein, the term “conformational epitope” refers to an epitope where the amino acid residues take a particular three-dimensional structure. Antibodies which specifically bind a conformational epitope recognize the spatial arrangement of the amino acid residues of that conformational epitope. In some embodiments, a binding molecule (e.g., an antibody) binds a conformational epitope comprising an amino acid sequence with greater affinity or specificity than to the same amino acid sequence in a linear form.

A conformational epitope-containing peptide of the present invention can include a glycine residue located at either end of the SNK epitope sequence (GSNK (SEQ ID NO: 4) or SNKG (SEQ ID NO: 5)). A peptide can include glycine residues at both ends of the SNK epitope sequence (GSNKG (SEQ ID NO: 2)). The glycine residue(s) can have limited or no contribution to the immunogenicity of the conformational epitope, and/or can relieve some steric tension inherent in the cyclization of the peptide. The peptide can include a glycine following lysine closer to the C terminus and a cysteine closer to the N terminus of the sequence (CSNKG (SEQ ID NO: 6) or CGSNKGC (SEQ ID NO: 7) or CCGSNKGC (SEQ ID NO: 8) or CGSNKGG (SEQ ID NO: 3)). A conformational epitope can further include a cysteine followed by a native glycine on the N-terminal and a native glycine and a second glycine on the C terminal end (CGSNKGG (SEQ ID NO: 3)). A conformation epitope can further include an N-terminal acetylated cysteine, followed by an additional cysteine and a native glycine and a cysteine at the C terminal end. A conformational epitope can also comprise a combination of the aforementioned properties.

Conformational epitope-containing peptides can comprise any standard (or natural) amino acids, non-standard amino acids, and/or amino acid analogues. Standard amino acids also include selenocysteine and pyrrolysine.

Non-standard amino acids can be naturally occurring or non-naturally occurring. Non-standard amino acids include any amino acid that can be incorporated into a polypeptide or result from modification of a natural amino acid. Several naturally occurring non-standard amino acids are known in the art, such as 4-hydroxyproline, 5-hydroxylysine, 3-methylhistidine, N-acetylserine, etc. In the present disclosure, an amino acid can be an L-amino acid or a D-amino acid. Amino acids in the present disclosure can be subject to any suitable modification, such as methylation, acetylation and/or phosphorylation.

Alteration can comprise replacing one or more amino acid residue(s) with a non-naturally occurring or non-standard amino acid, modifying one or more amino acid residue into a non-naturally occurring or non-standard form, or inserting one or more non-naturally occurring or non-standard amino acid into the sequence. Non-standard amino acids and amino acid analogues can be incorporated into a peptide during synthesis or by modification or by replacement of a natural amino acid after synthesis of a peptide.

An amino acid can be replaced by another amino acid on the basis of their structure and the general chemical characteristics of their R groups (side-chains). For example, an aliphatic amino acid can be replaced by another aliphatic amino acid; a hydroxyl or sulfur-containing amino acid can be replaced by another hydroxyl or sulfur-containing amino acid; a cyclic amino acid can be replaced by another cyclic amino acid; an aromatic amino acid can be replaced by another aromatic amino acid, a basic amino acid can be replaced by another basic amino acid; an acid amino acid can be replaced by another acid amino acid, etc. Alterations can comprise modifying an L-amino acid into, or replacing it with, a D-amino acid.

In one embodiment, the conformational epitope comprises an amino acid sequence of SNK in a cyclic constrained configuration or in a cyclic peptide. In one aspect, the conformational epitope comprises an amino acid sequence of at least SNK.

In some embodiments, a conformational epitope comprises or consists of an amino acid sequence in a cyclic constrained configuration or in a cyclic peptide wherein the amino acid sequence is selected from of GSNKG (SEQ ID NO: 2); CGSNKGG (SEQ ID NO: 3); GSNK (SEQ ID NO: 4); SNKG (SEQ ID NO: 5); CSNKG (SEQ ID NO: 6); CGSNKGC (SEQ ID NO: 7); CCGSNKGC (SEQ ID NO: 8) and/or GGSNKGC (SEQ ID NO: 9). In some embodiments, a conformational epitope comprises or consists of an amino acid sequence of at least GSNKG (SEQ ID NO: 2); CGSNKGG (SEQ ID NO: 3); GSNK (SEQ ID NO: 4); SNKG (SEQ ID NO: 5); CSNKG (SEQ ID NO: 6); CGSNKGC (SEQ ID NO: 7); CCGSNKGC (SEQ ID NO: 8) and/or GGSNKGC (SEQ ID NO: 9).

The conformational epitope of an antigenic peptide can comprise amino acid residues corresponding to residues 25 to 29 of oligomeric Aβ (1-40) or oligomeric Aβ (1-42).

The conformational epitope can comprise polar/charged amino acid residues that are structurally constrained corresponding to the solvent-exposed amino acid residues located at the surface of Aβ oligomers.

As used herein, the term “cSNK” refers to a cyclic peptide comprising at least SNK, and can be any peptide described herein.

Methods of Treatment, Pharmaceutical Compositions, and Kits

As used herein, the term “treatment,” “treating,” or “ameliorating” refers to either a therapeutic treatment or prophylactic/preventative treatment. A treatment is therapeutic if at least one symptom of disease in an individual receiving treatment improves or a treatment can delay worsening of a progressive disease in an individual, or prevent onset of additional associated diseases.

In some embodiments, antibodies or antigen-binding fragments thereof have in vitro and/or in vivo therapeutic, prophylactic, and/or diagnostic utilities. For example, these antibodies can be administered to cells in culture, e.g., in vitro or ex vivo, or to a subject, e.g., in vivo, to diagnose or prevent an amyloid disease such as Alzheimer's disease, inhibit or delay the onset of the disease, or slow progression of the disease. The antibodies or antigen-binding fragments thereof disclosed herein can also be used in the study and research of diseases, such as amyloid diseases and those involving oligomeric Aβ. Amyloid diseases include, but are not limited to, Alzheimer's disease, Down's syndrome, dementia pugilistica, multiple system atrophy, inclusion body myositosis, hereditary cerebral hemorrhage with amyloidosis of the Dutch type, Nieman-Pick disease type C, cerebral β-amyloid angiopathy, dementia associated with cortical basal degeneration, type 2 diabetes, chronic inflammation, malignancy and Familial Mediterranean Fever, multiple myeloma and B-cell dyscrasias, the prion diseases, Creutzfeldt-Jakob disease, Gerstmann-Straussler syndrome, kuru, scrapie, the amyloidosis associated with carpal tunnel syndrome, senile cardiac amyloidosis, familial amyloidotic polyneuropathy, and the amyloidosis associated with endocrine tumors.

Alzheimer patients receiving one or more compositions disclosed herein can be in the early, middle or late stages of the disease progression, with mild, moderate or severe symptoms. In other cases, individuals suspected of beginning to develop Alzheimer's disease or considered at risk of developing this disease can also receive such treatment, so that their progression towards onset of the disease can be halted or reversed, or their risk of developing the disease can be diminished or eliminated. In other words, the anti-Alzheimer treatment can be applied as a method of preventing Alzheimer's disease or inhibiting or delaying the onset and/or progression of the disease in at-risk individuals with no or only suspected symptoms. U.S. Pat. No. 8,066,993. In some embodiments, a composition, antibody or fragment thereof is administered to a patient, wherein the patient has reduced levels of their own antibodies against an SNK containing conformational epitope located in Aβ oligomers. In some embodiments, a patient or subject's levels of Aβ and/or anti-Aβ-oligomer antibodies (or antigen-binding fragment thereof) are monitored, e.g., before, during and or after treatment.

Compositions disclosed herein can be used for prophylaxis and/or treatment of Alzheimer's disease. A pharmaceutically effective amount of a pharmaceutical composition can be administered to an individual diagnosed with Alzheimer's disease. A pharmaceutically effective amount of a pharmaceutical composition can be administered to an individual at risk for developing Alzheimer's disease or to a person with an unknown risk. An effective amount to be administered to the subject can be determined by a physician with consideration of individual differences in age, weight, disease severity, dose and frequency of administration, and individual response to the therapy.

The antibody or antigen-binding fragment thereof can be administered alone or in combination with another therapeutic agent, e.g., a second monoclonal or polyclonal antibody or antigen-binding fragment thereof, or other therapeutic agents to treat Alzheimer's disease.

The pharmaceutical compositions can contain the antibodies together with one or more other active agents. Alternatively, a composition of the invention can be administered consecutively, simultaneously or in combination with one or more other active agents. Non-limiting examples of the active agents that can be used in combination include an acetylcholinesterase inhibitor (such as tacrine, rivastigmine, galantamine or donepezil) or an NMDA receptor antagonist (such as memantine). Other active agents that can be used in combination include those that are useful for treating Alzheimer's disease or related dementias. For example, an antibody or antigen-binding fragment thereof disclosed herein can be co-formulated coadministered and/or sequentially administered with one or more additional antibodies that bind other targets.

As used herein, the term “pharmaceutically acceptable” refers to molecular entities and compositions that do not produce allergic or other serious adverse reactions in a majority of subjects when administered, e.g., using routes well known in the art. Molecular entities and compositions approved by a regulatory agency of the Federal or a state government or listed in the U.S. Pharmacopeia or other generally recognized pharmacopeia for use in animals, and more particularly in humans are considered to be “pharmaceutically acceptable.”

The disclosure also provides compositions containing an antibody or antigen-binding fragment thereof described herein. The composition can contain an isolated nucleic acid encoding the antibody or antigen-binding fragment thereof. Compositions can also contain a pharmaceutically acceptable carrier. Pharmaceutically acceptable carriers include any and all solvents, dispersion media, isotonic and absorption delaying agents, and the like that are physiologically compatible. The pharmaceutical composition can be used for preventing the onset or reducing the severity or duration of Alzheimer's disease. As discussed above, antibodies or antigen-binding fragments thereof are specific to a conformational epitope having an amino acid sequence comprising at least SNK. The invention also includes a therapeutically effective amount of a humanized or chimeric antibody, or antigen binding fragment thereof, that reduces the propagation of Aβ monomers to Aβ oligomers

The disclosure also provides methods of treating or preventing Alzheimer's disease in a subject by administering to the subject a pharmaceutical composition containing an antibody or antigen-binding fragment thereof disclosed herein in an amount effective to treat or prevent Alzheimer's disease.

In some embodiments, an antibody, or antigen-binding fragment thereof as provided herein is administered to a subject, wherein the antibody or fragment thereof can bind to a cyclic peptide comprising the amino acid sequence SNK, wherein the K (Lysine) is solvent-accessible.

In certain embodiments, a composition can be administered to a subject at a dose ranging from about 1 μg to 1 mg/kg body weight, about 10 mg to 800 mg/kg body weight, about 20 mg to 600 mg/kg body weight, about 30 mg to 500 mg/kg body weight, about 10 mg to 400 mg/kg body weight, about 20 mg to 400 μg/kg body weight, about 60 mg to 100 mg/kg body weight, about 10 mg to 200 mg per kg body weight, about 100 mg to 200 mg/kg body weight, about 50 mg/kg body weight, or about 100 mg/kg body weight. The dose can also range from about 10 mg/kg of body weight to about 5 g/kg body weight, about 5 mg/kg of body weight to about 2 g/kg body weight, about 50 mg/kg of body weight to about 4 g/kg body weight, about 100 mg/kg of body weight to about 3 g/kg body weight, about 0.1 g/kg body weight to about 1 g/kg body weight, about 0.2 g/kg body weight to about 0.8 g/kg body weight, about 0.2 g/kg of body weight to about 4 g/kg body weight, about 10 mg/kg of body weight to about 50 mg/kg body weight, about 0.2 g/kg body weight, about 0.4 g/kg body weight, about 0.8 g/kg body weight, about 5 mg/kg body weight to about 500 mg/kg body weight, at least about 10 mg/kg body weight, at least about 15 mg/kg body weight, at least about 20 mg/kg body weight, at least about 25 mg/kg body weight, at least about 30 mg/kg body weight or at least 50 mg/kg body weight, up to about 100 mg/kg body weight, up to about 150 mg/kg body weight, up to about 200 mg/kg body weight, up to about 250 mg/kg body weight, up to about 300 mg/kg body weight, or up to about 400 mg/kg body weight. In other embodiments, the doses of the immunoglobulin can be greater or less.

Using a mass/volume unit, a composition can be administered to a subject at a dose ranging from about 0.1 mg/ml to about 2000 mg/ml, or any amount therebetween, for example 0.1, 0.5, 1, 2, 5, 10, 15, 20, 25, 30, 35, 40, 10 50, 60, 70, 80, 90, 100, 120, 140, 160 180, 200, 250, 500, 750, 1000, 1500, 2000 mg/ml, or any amount therebetween; or from about 1 mg/ml to about 2000 mg/ml, or any amount therebetween, for example, 1.0, 2.0, 5.0, 10.0, 15.0, 20.0, 25.0, 30.0, 35.0, 40.0, 50.0 60.0, 70.0, 80.0, 90.0, 100, 120, 140, 160 180, 200, 250, 500, 750, 1000, 1500, 2000, mg/ml or any amount therebetween; or from about 10 mg/ml to about 1000 mg/ml or any amount 15 therebetween, for example, 10.0, 15.0, 20.0, 25.0, 30.0, 35.0, 40.0, 50.0 60.0, 70.0, 80.0, 90.0, 100, 120, 140, 160 180, 200, 250, 500, 750, 1000 mg/ml, or any amount therebetween; or from about 30 mg/ml to about 1000 mg/ml or any amount therebetween, for example 30.0, 35.0, 40.0, 50.0 60.0, 70.0, 80.0, 90.0, 100, 120, 140, 160 180, 200, 250, 500, 750, 1000 mg/ml.

The data obtained from the cell culture assays and animal studies can be used in formulating a range of dosage for use in humans or other animal species. In one embodiment, the dosage of such compounds lies within a range of circulating concentrations that include the ED₅₀ with little or no toxicity. The dosage can vary within this range depending upon the dosage form employed and the route of administration utilized. In another embodiment, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Sonderstrup, Springer, Sem. Immunopathol. 25: 35-45, 2003. Nikula et al., Inhal. Toxicol. 4(12): 123-53, 2000.

The composition is formulated to contain an effective amount of an antibody or antigen-binding fragment thereof, wherein the amount depends on the subject to be treated, the condition to be treated and/or the severity of the disease or symptoms. In one embodiment, an antibody or antigen-binding fragment thereof is administered at a dose ranging from about 0.01 mg to about 10 g, from about 0.1 mg to about 9 g, from about 1 mg to about 8 g, from about 1 mg to about 7 g, from about 5 mg to about 6 g, from about 10 mg to about 5 g, from about 20 mg to about 1 g, from about 50 mg to about 800 mg, from about 100 mg to about 500 mg, from about 0.01 mg to about 10 g, from about 0.05 mg to about 1.5 mg, from about 10 mg to about 1 mg protein, from about 30 mg to about 500 mg, from about 40 pg to about 300 pg, from about 0.1 mg to about 200 mg, from about 0.1 mg to about 5 mg, from about 5 mg to about 10 mg, from about 10 mg to about 25 mg, from about 25 mg to about 50 mg, from about 50 mg to about 100 mg, from about 100 mg to about 500 mg, from about 500 μg to about 1 mg, from about 1 mg to about 2 mg. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy.

The duration of administration can vary: it can range from about 10 minutes to about 1 day, from about 30 minutes to about 20 hours, from about 1 hour to about 15 hours, from about 2 hours to about 10 hours, from about 3 hours to about 8 hours, from about 4 hours to about 6 hours, from about 1 day to about 1 week, from about 2 weeks to about 4 weeks, from about 1 month to about 2 months, from about 2 months to about 4 months, from about 4 months to about 6 months, from about 6 months to about 8 months, from about 8 months to about 1 year, from about 1 year to about 2 years, or from about 2 years to about 4 years, or more. The duration of administration can be about 1 month, about 3 months, about 6 months, about 1 year, about 18 months, about 2 years, about 5 years, or about 10 years. In one embodiment, the treatment can last the remainder of a subject's natural life.

Compositions can be administered in a single dose treatment or in multiple dose treatments on a schedule and over a time period appropriate to the age, weight and condition of the subject, the particular composition used, and the route of administration. In one embodiment, a single dose of the composition according to the invention is administered. In other embodiments, multiple doses are administered. The frequency of administration can vary depending on any of a variety of factors, e.g., severity of the symptoms, degree of immunoprotection desired, whether the composition is used for prophylactic or curative purposes, etc. For example, the composition according to the invention is administered about once per day, once per month, twice per month, three times per month, about once every other month, every other week (qow), once per week (qw), twice per week (biw), three times per week (tiw), four times per week, five times per week, six times per week, every other day (qod), daily (qd), twice a day (qid), three times a day (tid), about once every 6 months, about once a year, about once every 2 years, or about once every 5 years, etc. The composition can also be administered in one or more doses per day.

Effectiveness of the treatment can be assessed during the entire course of administration after a certain time period, e.g., about every 3 months, about every 6 months, about every 9 years, about every year, etc. The administration schedule (dose and frequency) can be adjusted accordingly for any subsequent administrations. U.S. Pat. Nos. 8,066,993 and 7,968,293.

Compositions or nucleic acids, polypeptides, or antibodies of the invention can be delivered alone or as pharmaceutical compositions by any routes known in the art, e.g., systemically, regionally, or locally; by intra-arterial, intrathecal (IT), intramuscular, intravenous (IV), parenteral, intra-pleural cavity, topical, oral, enteral, intranasal, intrapulmonary or inhalational, subcutaneous, intra-tracheal, transdermal, or transmucosal. Actual methods for preparing parenterally administrable compositions will be known or apparent to those skilled in the art and are described in detail. Bai, J. Neuroimmunol. 80: 65-75, 1997. Warren, J. Neurol. Sci. 152: 31-38, 1997. Tonegawa, J. Exp. Med. 186: 507-515, 1997. When administering the compositions by injection, the administration can be by continuous infusion or by single or multiple bolus injections.

In certain embodiments, an antibody or fragment thereof as provided herein can be linked to a half-life extending vehicle known in the art. Such vehicles include, but are not limited to, the Fc domain, polyethylene glycol (PEG), and dextran. Such vehicles are described, e.g., in U.S. application Ser. No. 09/428,082 and WO 99/25044. In some embodiments, an antibody or binding fragment thereof can be glyco-modified (e.g., deglycosylated, etc.) with improved effector function profile.

Pharmaceutical techniques can also be employed to control the duration of action of the compositions/preparations of the present invention. Control release preparations can be prepared through the use of polymers to complex, encapsulate, or absorb the antibodies. WO1999040939. In certain embodiments, the composition takes the form of, for example, implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

The pharmaceutical compositions provided herein can be in a variety of forms. These include, for example, liquid, semi-solid and solid dosage forms, such as liquid solutions (e.g., injectable and infusible solutions), dispersions or suspensions, tablets, pills, powders, liposomes and suppositories. The form can depend on the intended mode of administration and therapeutic application. The liquid formulation can be administered directly. The lyophilized powder formulation can be reconstituted in a physiologically compatible medium before administration.

The pharmaceutical compositions provided herein can be prepared as injectables, either as liquid solutions or suspensions, or as solid forms which are suitable for solution or suspension in liquid vehicles prior to injection. The composition can also be prepared in solid form, emulsified or the active ingredient encapsulated in liposome vehicles or other particulate carriers used for sustained delivery. For example, the composition can be in the form of an oil emulsion, water-in-oil emulsion, water-in-oil-in-water emulsion, site-specific emulsion, long-residence emulsion, stickyemulsion, microemulsion, nanoemulsion, liposome, microparticle, microsphere, nanosphere, nanoparticle and various natural or synthetic polymers, such as nonresorbable impermeable polymers such as ethylenevinyl acetate copolymers and Hytrel® copolymers, swellable polymers such as hydrogels, or resorbable polymers such as collagen and certain polyacids or polyesters such as those used to make resorbable sutures, that allow for sustained release of an antibody (or antigen binding fragment thereof) or a vaccine.

By way of example, intravenously injectable immunologlobulin preparations can contain an immunologlobulin distributed in a physiologically compatible medium. Suitable medium for compositions can be sterile water for injection (WFI) with or without isotonic amounts of sodium chloride. For example, diluents include sterile WFI, sodium chloride solution (see Gahart, B. L. & Nazareno, A. R., Intravenous Medications: a handbook for nurses and allied health professionals, p. 516-521, Mosby, 1997).

The immunoglobulin concentration in the pharmaceutical composition can range from about 0.1% (w/w) to about 30% (w/w), from about 0.5% (w/w) to about 20% (w/w), from about 1% (w/w) to about 15% (w/w), from about 2% (w/w) to about 3% (w/w), or from about 5% (w/w) to about 10% (w/w).

In certain embodiments, compositions can be orally administered, for example, with an inert diluent or an assimilable edible carrier. The compound (and other ingredients, if desired) can also be enclosed in a hard or soft shell gelatin capsule, compressed into tablets, or incorporated directly into the subject's diet. For oral therapeutic administration, the compounds can be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation. Compositions for oral administration via tablet, capsule or suspension are prepared using adjuvants including sugars, such as lactose, glucose and sucrose; starches such as corn starch and potato starch; cellulose and derivatives thereof, including sodium carboxymethylcellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil and corn oil; polyols such as propylene glycol, glycerine, sorbital, mannitol and polyethylene glycol; agar; alginic acids; water; isotonic saline and phosphate buffer solutions. Wetting agents, lubricants such as sodium lauryl sulfate, stabilizers, tableting agents, anti-oxidants, preservatives, coloring agents and flavoring agents can also be present.

Creams, lotions and ointments can be prepared for topical application using an appropriate base such as a triglyceride base. Such creams, lotions and ointments can also contain a surface active agent. Aerosol formulations, for example, for nasal delivery, can also be prepared in which suitable propellant adjuvants are used. Other adjuvants can also be added to the composition regardless of how it is to be administered, for example, anti-microbial agents can be added to the composition to prevent microbial growth over prolonged storage periods. Therapeutic compositions typically must be sterile and stable under conditions of manufacture and storage.

In some embodiments, antibodies or antigen-binding fragments thereof are formulated into compositions for delivery to a mammalian subject. The composition is administered alone, and/or mixed with a pharmaceutically acceptable vehicle or excipient. Suitable vehicles are, for example, water, saline, dextrose, glycerol, ethanol, or the like, and combinations thereof. In addition, the vehicle can contain minor amounts of auxiliary substances such as wetting or emulsifying agents, pH buffering agents, or adjuvants. The compositions of the invention can also include ancillary substances, such as pharmacological agents, cytokines, or other biological response modifiers. Methods of preparing the formulations are known, or will be apparent, to those skilled in the art. See, e.g., Remington's Pharmaceutical Sciences, Mack Publishing Company, Easton, Pa., 21st edition.

In some embodiments, antibodies or antigen-binding fragments thereof can be combined with a pharmaceutically acceptable carrier. Pharmaceutically acceptable carriers can contain a physiologically acceptable compound that acts to, e.g., stabilize, or increase or decrease the absorption or clearance rates of antibodies or antigen-binding fragment thereof. Physiologically acceptable compounds can include, e.g., carbohydrates, such as glucose, sucrose, or dextrans, antioxidants, such as ascorbic acid or glutathione, chelating agents, low molecular weight proteins, detergents, liposomal carriers, or excipients or other stabilizers and/or buffers. Other physiologically acceptable compounds include wetting agents, emulsifying agents, dispersing agents or preservatives. See e.g., the 21st edition of Remington's Pharmaceutical Science, Mack Publishing Company, Easton, Pa. (“Remington's”).

In one aspect, antibodies or antigen-binding fragments thereof are dissolved in a pharmaceutically acceptable carrier, e.g., an aqueous carrier. Examples of aqueous solutions include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate.

Solid formulations can also be used for formulation of the antibodies or compositions of the invention. They can be formulated as, e.g., pills, tablets, powders or capsules. For solid compositions, conventional solid carriers can be used which include, e.g., mannitol, lactose, starch, magnesium stearate, sodium saccharin, talcum, cellulose, glucose, sucrose, magnesium carbonate, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. Sayani, Crit. Rev. Ther. Drug Carrier Syst. 13: 85-184, 1996. For topical, transdermal administration, the agents are formulated into ointments, creams, salves, powders and gels. Transdermal delivery systems can also include, e.g., patches.

For inhalation, compositions disclosed herein can be delivered using any system known in the art, including dry powder aerosols, liquids delivery systems, air jet nebulizers, propellant systems, and the like. Patton, Biotechniques 16: 141-143, 1998. In some embodiments, product and inhalation delivery systems for polypeptide macromolecules by, e.g., Dura Pharmaceuticals (San Diego, Calif.), Aradigrn (Hayward, Calif.), Aerogen (Santa Clara, Calif.), Inhale Therapeutic Systems (San Carlos, Calif.), and the like are utilized. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In some embodiments, compositions are administered in sustained delivery or sustained release mechanisms. For example, biodegradable microspheres or capsules or other biodegradable polymer configurations capable of sustained delivery of a peptide can be included in the formulations of the invention (see, e.g., Putney, Nat. Biotechnol. 16: 153-157, 1998).

In one aspect, the compositions are prepared with carriers that will protect the peptide against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. U.S. Pat. No. 4,522,811.

In one aspect, the pharmaceutical formulations comprising nucleic acids, polypeptides, or antibodies provided herein can be incorporated in lipid monolayers or bilayers, e.g., liposomes. U.S. Pat. Nos. 6,110,490; 6,096,716; 5,283,185 and 5,279,833. Aspects of the invention also provide formulations in which nucleic acids, peptides or polypeptides of the invention have been attached to the surface of the monolayer or bilayer. For example, peptides can be attached to hydrazide-PEG-(distearoylphosphatidyl) ethanolamine-containing liposomes (see, e.g., Zalipsky, Bioconjug. Chem. 6: 705-708, 1995). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla, J. Pharm. Sci. 85: 5-8, 1996), transmucosally, or orally. The invention also provides pharmaceutical preparations in which the nucleic acid, peptides and/or polypeptides of the invention are incorporated within micelles and/or liposomes (see, e.g., Suntres, J. Pharm. Pharmacol. 46: 23-28, 1994; Woodle, Pharm. Res. 9: 260-265, 1992). Liposomes and liposomal formulations can be prepared according to standard methods and are also well known in the art. Akimaru, Cytokines Mol. Ther. 1: 197-210, 1995. Alving, Immunol. Rev. 145: 5-31, 1995. Szoka, Ann. Rev. Biophys. Bioeng. 9: 467, 1980. U.S. Pat. Nos. 4,235,871; 4,501,728 and 4,837,028.

It is advantageous to formulate parenteral or oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

The invention also includes an article of manufacture comprising packaging material and a pharmaceutical composition. The composition comprises a pharmaceutically acceptable carrier and a pharmaceutically effective amount of the antibody or antigen-binding fragment thereof as described above. The packaging material can be labeled to indicate that the composition is useful to treat or prevent Alzheimer's disease. The packaging material can be any suitable material generally used to package pharmaceutical agents including, for example, glass, plastic, foil and cardboard.

In another embodiment, a kit for detecting the level of oligomeric Aβ in a biological sample is provided. The kit comprises an antibody or antigen-binding fragment thereof as described above, along with instructions for use of the antibody or antigen-binding fragment thereof.

The antibody or antigen-binding fragment thereof can further be coupled to a detection reagent. Examples of detection reagents include secondary antibodies, such as an anti-human antibody, an anti-mouse antibody, an anti-rabbit antibody or the like. Such secondary antibodies can be coupled with an enzyme that, when provided with a suitable substrate, provides a detectable colorimetric or chemiluminescent reaction. The kit can further comprise reagents for performing the detection reaction, including enzymes such as proteinase K, blocking buffers, homogenization buffers, extraction buffers, dilution buffers or the like.

Additional components of the kits can include one or more of the following: instructions for use; another therapeutic agent, an agent useful for coupling an antibody to a label or therapeutic agent, other reagents, or other materials for preparing the antibody for administration; pharmaceutically acceptable carriers; and devices or other materials for administration to a subject.

Instructions for use can include instructions for therapeutic applications, suggested dosages, dose intervals, modes of administration, and/or methods for immunological screening or testing, etc. Other instructions can include instructions on coupling of the antibody to a label or a therapeutic agent, or for purification of a conjugated antibody, e.g., from unreacted conjugation components.

A kit can contain at least one nucleic acid encoding the antibodies or fragment thereof, and instructions for expression of the nucleic acids. Other possible components of the kit include expression vectors and cells.

### EXAMPLES

### Example 1

### Cloning, Characterization, and Sequencing of the Murine Monoclonal Antibody 5E3

Murine monoclonal antibody 5E3 was raised against a constrained cyclic peptide comprising residues 25-29 (GSNKG, SEQ ID NO: 2) of amyloid beta (Aβ). Murine 5E3 mAb selectively recognizes a conformational epitope on the oligomeric form of Aβ, and shows little or no binding to monomeric or linear Aβ, to Aβ fibrils, or to amyloid precursor protein (APP). See PCT Publication No. WO2011/106885, which is incorporated herein by reference in its entirety. Murine 5E3 mAb was subcloned, and clonal isolates were tested for binding to disulfide-linked cyclic peptides comprising the tripeptide SNK (e.g., SEQ ID NOs 2-9) by ELISA and western blot (data not shown). The absence of binding to Aβ fibrils was demonstrated by immunohistochemistry on frontal cortex brain sections from AD and age-matched control patients. These were probed with murine 5E3 mAb and the anti-AP mAb2C8 (raised against N-terminal residues 1-16 of Aβ and recognizes fibril insoluble plaque). Fibrillar plaques were clearly detected by 2C8 in the AD brains, whereas murine 5E3 mAb probed sections showed an absence of signal (data not shown).

### Example 2

### V Gene Sequencing

RNA was isolated from the 5E3 parental hybridoma clonal cell line using the RNAeasy Mini Kit. RT-PCR was used to isolate cDNAs encoding the heavy and light chain variable domains (VH and VL) of 5E3. The cDNAs were cloned and sequenced using standard techniques. The cDNA sequences encoding the VL and VH of 5E3 are presented as SEQ ID NO: 10 and SEQ ID NO: 15, respectively, and the amino acid sequences are shown as SEQ ID NO: 11 and SEQ ID NO: 16, respectively. The amino acid sequences of the three light chain complementarity determining regions LCDR1, LCDR2, and LCDR3 are presented as SEQ ID NO: 12, SEQ ID NO: 13, and SEQ ID NO: 14, respectively, and the HCDR1, HCDR2, and HCDR3 regions are presented as SEQ ID NO: 17, SEQ ID NO: 18, and SEQ ID NO: 19, respectively.

### Example 3

### Humanization of Murine 5E3

Three humanized IgG/k versions of the 5E3 murine mAb were created as follows. Human germline heavy and light chain variable domains with maximum identity alignment with the murine sequences were identified in the NCBI databases for use as identify acceptor frameworks. The human germline alleles selected were hIGKV1D-16-01/hIGHV1-3-01 (VH chain) and hIGKV1-16-01/hIGKJ4-01 (VK chain). These best matching human germline alleles were used as an acceptor framework for grafting the CDRs. All 6 CDRs (SEQ ID NOs 12-14 and 17-19) corresponding to heavy and light chains were inserted into the human framework regions. The cdr5E3 VL and VH regions are presented as SEQ ID NOs 60 and 61 (nucleotide and amino acid sequences of cdr5E3 VL) and SEQ ID NOs 62 and 63 (nucleotide and amino acid sequences of cdr5E3 VH).

Other residues were changed or maintained due to surface exposure or involvement in folding or interchain contacts, respectively. This resembles the “superhumanization” approach where CDR matching rather than total framework is used in a variation of the use of germline sequences as acceptor frameworks. In the case of Tan et al., J. Immunol. 2002, 169:1119-1125, the authors used the CDR sequences and tried to match the so called canonical classes of CDRs based upon the Kabat classification system. However, because particular CDRs are germline encoded and particular canonical conformations tend to be found in certain frameworks, the “Superhumanization” method of choosing acceptor frameworks does not in all cases result in the selection of a different candidate acceptor framework. It is empirical and remains to be tested for multiple mAb specificities. This is in part because the straight-up alignment of frameworks for identity inherently encompasses the CDRs as well in the comparison.

Antibodies hu5E3 and rehu5E3 “Human engineered” were generated using a strategy most similar to the “human engineering” strategy used by Studnicka et al. to humanize a murine mAb to CD5 (Studnicka et al, Protein Eng. 1994 June; 7(6):805-14). Essentially, the closest human germline allele for 5E3 mAbs VH and Vk were identified, individually, and designed for use as acceptor frameworks, resulting in the VH and VL sequences of hu5E3. These sequences are presented as SEQ ID NOs 64 and 65 (nucleotide and amino acid sequences of hu5E3 VL) and SEQ ID NOs 66 and 67 (nucleotide and amino acid sequences of hu5E3 VH). The rehu5E3 mAb was further resurfaced by substitution(s) made on surface exposed amino acids to correspond to the adopted human frameworks without disruption of the CDRs. These sequences are presented as SEQ ID NOs 68 and 69 (nucleotide and amino acid sequences of rehu5E3 VL) and SEQ ID NOs 70 and 71 (nucleotide and amino acid sequences of rehu5E3 VH).

These VH and VL regions were cloned into vectors for expression as full-sized humanized antibodies having human IgG1, IgG2 or IgG4 constant regions. At the same time, the VH and VL regions of the parent murine 5E3 antibody were cloned into human constant region vectors for expression as mouse-human chimeric antibodies.

### Example 4

### Transient Expression and Purification of Humanized 5E3 mAbs

Humanized 5E3 mAbs were produced by transient transfections in CHO-S or CHOK1S-V cells. One day prior to transfection, CHO—S or CHOK1S-V cells were counted using a Haemocytometer in the presence of Trypan Blue, then passaged into transfection medium (DMEM/F12 supplemented with 10% FBS and L-Glutamine) at a concentration of 6-8×10⁵ cells/nil and incubated 24 hours at 37° C., 8% CO₂ and 100 rpm. Freestyle Max Transfection Agent was diluted 1/16 in Optimem before being added to 312.5 ng of the appropriate DNA also diluted in Optimem. DNA/Freestyle Max Transfection Agent mix was incubated at room temperature for 20 minutes and added to 250×10⁶ CHO-S cells in DMEM/F12+10% FBS+5 mM L-Glutamine that had been treated for 3 hours with 1% DMSO.

The culture was harvested after incubation at 37° C./5% CO₂/125 rpm by centrifuging the culture at 2500 rpm for 30 minutes, removing the supernatant and filtering it through a 0.22 μM bottle top filter. The supernatant was concentrated by spin cell concentrator equipped with a 30 kDa membrane to a final volume of ˜400 mL. The concentrated supernatant was purified by Protein G purification on the FPLC. The purified sample was buffer exchanged by spin-cell concentrator equipped with a 30 kDa membrane into D-PBS and concentrated down to a final volume of 1-2 mL. The final protein concentration was determined by BCA using the Pierce BCA Kit.

### Example 5

### Qualitative Assessment of Interaction Between Humanized 5E3 mAbs and a cSNK:BSA Conjugate

Biolayer interferometry (Octet Red92, ForteBio) was used to qualitatively/quantitatively assess the interactions between the humanized and chimeric versions of 5E3, expressed and purified as described in Example 4, and a cyclized SNK peptide conjugated to a BSA carrier (cSNK:BSA conjugate).

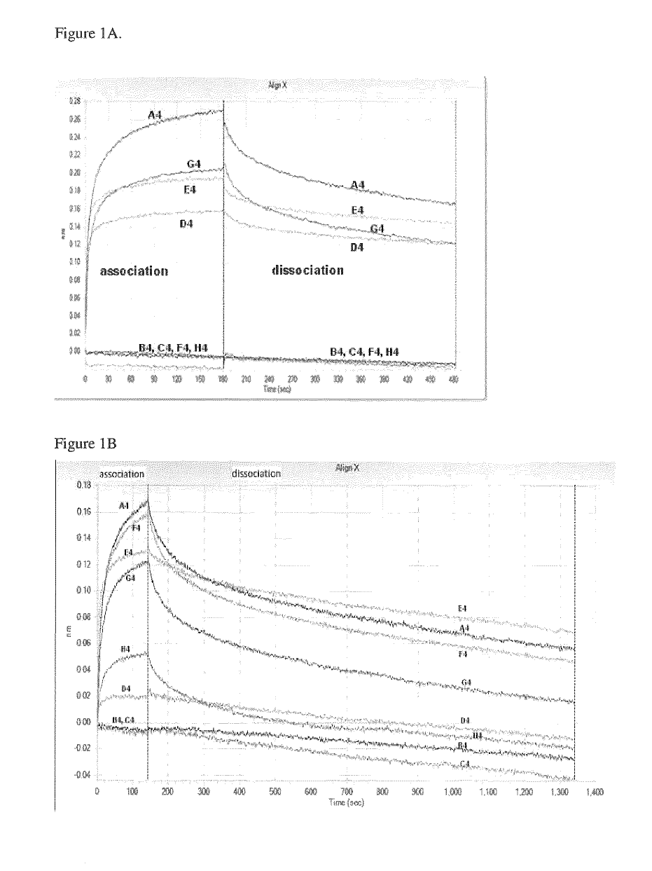

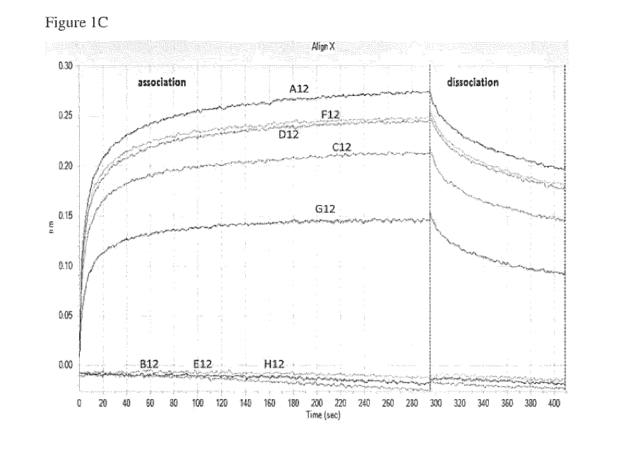

Each mAb (40 μg/mL) was coupled/loaded onto either of two sensors: AMC (anti-Fc mouse) or AHC (anti-Fc human). Sensors were washed in PBST (PBS+0.1% triton) until a stable baseline was achieved. The sensors were then associated with cSNK:BSA conjugate (100 nM), followed by dissociation in PBST. As shown in FIG. 1A, in the association phase, a binding event (wavelength upshift) was detected between cSNK:BSA conjugate and hu5E3 (sensor G4). Binding between the conjugate and the following mAbs was not detected: cdr5E3 (sensor F4) and rehu5E3 (sensor H4). A second assay was performed on additional isolates (FIG. 1B). In this assay, binding events (wavelength upshift) were detected between cSNK:BSA conjugate and the following hu5E3 (IgG1) (sensor F4), hu5E3 (IgG2) (sensor G4), and rehu5E3 (IgG2) (sensor H4). A third assay was conducted with the mAbs in the context of human IgG4 isotypes (FIG. 1C). These results indicate that the humanized versions of murine 5E3 could recognize the cSNK epitope.

### Example 6

### cSNK Affinity of Humanized 5E3 mAbs

Biolayer interferometry (Octet Red92, ForteBio) was used to qualitatively/quantitatively assess the interactions between humanized versions of 5E3 and cSNK:BSA conjugate. To analyze affinity, sensors were washed in PBST (PBS+0.1% triton) until a stable baseline was achieved. MAbs were loaded onto sensors and then washed in PBST. Each sensor was associated with one of several cSNK:BSA standards (100 nM to 0 nM) and then washed in PBST. Data was processed statistically using ForteBio Data Analysis software to determine the strength of antigen-antibody binding (KD). For each mAb analyzed, a 2:1 model provided the “best fit”.

Results are shown in Tables 1-6. For murine 5E3, the KD value was within the range of 3.30E-10 to 1.59E-09 (Table 1). For hu5E3-IgG1, the KD value was within the range of 4.63E-10 and 1.82E-09 (Table 2). For hu5E3-IgG2, the KD range was between 1.18E-9 to 6.39E-9 (Table 3). For rehu5E3-IgG2, the KD ranged between 1.28E-9 and 8.14E-9 (Table 4). For hu5E3-IgG4, the KD range was between 6.33E-10 to 3.04E-10 (Table 5). For rehu5E3-IgG4, the KD ranged between 5.57E-10 to 3.94E-9 (Table 6).

TABLE 1
Affinity analysis between murine 5E3 (40 ug/mL) and cSNK:BSA standards (using AMC sensors)

| Conc. | | | kon | kon | | kon2 | | kdis | kdis2 | kdis2 | Full | Full |
|---|---|---|---|---|---|---|---|---|---|---|---|---|
| (nM) | KD (M) | KD2 (M) | (1/Ms) | Error | kon2 | error | kdis (1/s) | Error | (1/s) | error | X\hat{ }2 | R\hat{ }2 |
| 100 | 3.30E−10 | 1.59E−09 | 1.29E+06 | 1.09E+04 | 1.42E+07 | 4.39E+05 | 4.28E−04 | 3.74E−06 | 2.26E−02 | 2.37E−04 | 0.0372 | 0.9978 |
| 50 | 3.30E−10 | 1.59E−09 | 1.29E+06 | 1.09E+04 | 1.42E+07 | 4.39E+05 | 4.28E−04 | 3.74E−06 | 2.26E−02 | 2.37E−04 | 0.0372 | 0.9978 |
| 25 | 3.30E−10 | 1.59E−09 | 1.29E+06 | 1.09E+04 | 1.42E+07 | 4.39E+05 | 4.28E−04 | 3.74E−06 | 2.26E−02 | 2.37E−04 | 0.0372 | 0.9978 |
| 12.5 | 3.30E−10 | 1.59E−09 | 1.29E+06 | 1.09E+04 | 1.42E+07 | 4.39E+05 | 4.28E−04 | 3.74E−06 | 2.26E−02 | 2.37E−04 | 0.0372 | 0.9978 |
| 6.25 | 3.30E−10 | 1.59E−09 | 1.29E+06 | 1.09E+04 | 1.42E+07 | 4.39E+05 | 4.28E−04 | 3.74E−06 | 2.26E−02 | 2.37E−04 | 0.0372 | 0.9978 |
| 3.13 | 3.30E−10 | 1.59E−09 | 1.29E+06 | 1.09E+04 | 1.42E+07 | 4.39E+05 | 4.28E−04 | 3.74E−06 | 2.26E−02 | 2.37E−04 | 0.0372 | 0.9978 |
| 1.56 | 3.30E−10 | 1.59E−09 | 1.29E+06 | 1.09E+04 | 1.42E+07 | 4.39E+05 | 4.28E−04 | 3.74E−06 | 2.26E−02 | 2.37E−04 | 0.0372 | 0.9978 |

TABLE 2
Affinity analysis between hu5E3-IgG1 (40 ug/mL) and
cSNK:BSA standards (using AHC sensors)

| | | | kon | kon | kon2 | kon2 |
|---|---|---|---|---|---|---|
| Conc. (nM) | KD (M) | KD2 (M) | (1/Ms) | Error | (1/Ms) | error |
| 100 | | | | | | |
| 50 | 4.63E−10 | 1.82E−09 | 1.78E+06 | 3.89E+04 | 6.56E+06 | 2.55E+05 |
| 25 | 4.63E−10 | 1.82E−09 | 1.78E+06 | 3.89E+04 | 6.56E+06 | 2.55E+05 |
| 12.5 | 4.63E−10 | 1.82E−09 | 1.78E+06 | 3.89E+04 | 6.56E+06 | 2.55E+05 |
| 6.25 | 4.63E−10 | 1.82E−09 | 1.78E+06 | 3.89E+04 | 6.56E+06 | 2.55E+05 |
| 3.13 | 4.63E−10 | 1.82E−09 | 1.78E+06 | 3.89E+04 | 6.56E+06 | 2.55E+05 |
| 1.56 | 4.63E−10 | 1.82E−09 | 1.78E+06 | 3.89E+04 | 6.56E+06 | 2.55E+05 |
| | | kdis | kdis2 | kdis2 | | Full |
| Conc. (nM) | kdis (1/s) | Error | (1/s) | error | Full X\hat{ }2 | R\hat{ }2 |
| 100 | | | | | | |
| 50 | 8.25E−04 | 1.74E−05 | 1.20E−02 | 2.58E−04 | 0.034395 | 0.990464 |
| 25 | 8.25E−04 | 1.74E−05 | 1.20E−02 | 2.58E−04 | 0.034395 | 0.990464 |
| 12.5 | 8.25E−04 | 1.74E−05 | 1.20E−02 | 2.58E−04 | 0.034395 | 0.990464 |
| 6.25 | 8.25E−04 | 1.74E−05 | 1.20E−02 | 2.58E−04 | 0.034395 | 0.990464 |
| 3.13 | 8.25E−04 | 1.74E−05 | 1.20E−02 | 2.58E−04 | 0.034395 | 0.990464 |
| 1.56 | 8.25E−04 | 1.74E−05 | 1.20E−02 | 2.58E−04 | 0.034395 | 0.990464 |

Note:
100 nM SNK:BSA standard was not included in data analysis

TABLE 3
Affinity analysis between hu5E3-IgG2 (40 ug/mL) and
cSNK:BSA standards (using AHC sensors)

| | | | kon | kon2 | kon | kon2 |
|---|---|---|---|---|---|---|
| Conc. (nM) | KD (M) | KD2 (M) | (1/Ms) | (1/Ms) | Error | error |
| 100 | 1.18E−09 | 6.39E−09 | 6.44E+05 | 5.28E+06 | 8.49E+03 | 1.36E+05 |
| 50 | 1.18E−09 | 6.39E−09 | 6.44E+05 | 5.28E+06 | 8.49E+03 | 1.36E+05 |
| 25 | 1.18E−09 | 6.39E−09 | 6.44E+05 | 5.28E+06 | 8.49E+03 | 1.36E+05 |
| 12.5 | 1.18E−09 | 6.39E−09 | 6.44E+05 | 5.28E+06 | 8.49E+03 | 1.36E+05 |
| 6.25 | 1.18E−09 | 6.39E−09 | 6.44E+05 | 5.28E+06 | 8.49E+03 | 1.36E+05 |
| 3.13 | 1.18E−09 | 6.39E−09 | 6.44E+05 | 5.28E+06 | 8.49E+03 | 1.36E+05 |
| 1.56 | | | | | | |
| | | kdis2 | kdis | kdis2 | | |
| Conc. (nM) | kdis (1/s) | (1/s) | Error | error | Full X\hat{ }2 | Full R\hat{ }2 |
| 100 | 7.62E−04 | 3.38E−02 | 5.02E−06 | 3.42E−04 | 0.014738 | 0.996671 |
| 50 | 7.62E−04 | 3.38E−02 | 5.02E−06 | 3.42E−04 | 0.014738 | 0.996671 |
| 25 | 7.62E−04 | 3.38E−02 | 5.02E−06 | 3.42E−04 | 0.014738 | 0.996671 |
| 12.5 | 7.62E−04 | 3.38E−02 | 5.02E−06 | 3.42E−04 | 0.014738 | 0.996671 |
| 6.25 | 7.62E−04 | 3.38E−02 | 5.02E−06 | 3.42E−04 | 0.014738 | 0.996671 |
| 3.13 | 7.62E−04 | 3.38E−02 | 5.02E−06 | 3.42E−04 | 0.014738 | 0.996671 |
| 1.56 | | | | | | |

TABLE 4
Affinity analysis between rehu5E3-IgG2 (40 ug/mL) and
cSNK:BSA standards (using AHC sensors)

| | | | kon | kon2 | kon | kon2 |
|---|---|---|---|---|---|---|
| Conc. (nM) | KD (M) | KD2 (M) | (1/Ms) | (1/Ms) | Error | error |
| 100 | 8.14E−09 | 1.28E−09 | 4.16E+06 | 6.98E+05 | 1.70E+05 | 2.50E+04 |
| 50 | 8.14E−09 | 1.28E−09 | 4.16E+06 | 6.98E+05 | 1.70E+05 | 2.50E+04 |
| 25 | 1.28E−09 | 8.14E−09 | 6.98E+05 | 4.16E+06 | 2.50E+04 | 1.70E+05 |
| 12.5 | 1.28E−09 | 8.14E−09 | 6.98E+05 | 4.16E+06 | 2.50E+04 | 1.70E+05 |
| 6.25 | 1.28E−09 | 8.14E−09 | 6.98E+05 | 4.16E+06 | 2.50E+04 | 1.70E+05 |
| 3.13 | | | | | | |
| 1.56 | | | | | | |
| | | kdis2 | kdis | kdis2 | | |
| Conc. (nM) | kdis (1/s) | (1/s) | Error | error | Full X\hat{ }2 | Full R\hat{ }2 |
| 100 | 3.38E−02 | 8.90E−04 | 5.55E−04 | 1.42E−05 | 0.008665 | 0.983215 |
| 50 | 3.38E−02 | 8.90E−04 | 5.55E−04 | 1.42E−05 | 0.008665 | 0.983215 |
| 25 | 8.90E−04 | 3.38E−02 | 1.42E−05 | 5.55E−04 | 0.008665 | 0.983215 |
| 12.5 | 8.90E−04 | 3.38E−02 | 1.42E−05 | 5.55E−04 | 0.008665 | 0.983215 |
| 6.25 | 8.90E−04 | 3.38E−02 | 1.42E−05 | 5.55E−04 | 0.008665 | 0.983215 |
| 3.13 | | | | | | |
| 1.56 | | | | | | |

TABLE 5
Affinity analysis between hu5E3-IgG4 (40 ug/mL) and
cSNK:BSA standards (using AHC sensors)

| Sample | Conc. | | | Kon | | kon2 | kon2 |
|---|---|---|---|---|---|---|---|
| ID | (nM) | KD (M) | KD2 (M) | (1/Ms) | kon Error | (1/Ms) | error |
| SNK:BSA | 100 | 6.33E−10 | 3.04E−09 | 8.68E+05 | 7.47E+03 | 7.52E+06 | 1.82E+05 |
| SNK:BSA | 50 | 6.33E−10 | 3.04E−09 | 8.68E+05 | 7.47E+03 | 7.52E+06 | 1.82E+05 |
| SNK:BSA | 25 | 6.33E−10 | 3.04E−09 | 8.68E+05 | 7.47E+03 | 7.52E+06 | 1.82E+05 |
| SNK:BSA | 12.5 | 6.33E−10 | 3.04E−09 | 8.68E+05 | 7.47E+03 | 7.52E+06 | 1.82E+05 |
| SNK:BSA | 6.25 | 6.33E−10 | 3.04E−09 | 8.68E+05 | 7.47E+03 | 7.52E+06 | 1.82E+05 |
| SNK:BSA | 3.13 | 6.33E−10 | 3.04E−09 | 8.68E+05 | 7.47E+03 | 7.52E+06 | 1.82E+05 |
| SNK:BSA | 1.56 | 6.33E−10 | 3.04E−09 | 8.68E+05 | 7.47E+03 | 7.52E+06 | 1.82E+05 |
| Sample | Conc. | Kdis | kdis | kdis2 | kdis2 | | |
| ID | (nM) | (1/s) | Error | (1/s) | error | Full X\hat{ }2 | Full R\hat{ }2 |
| SNK:BSA | 100 | 5.50E−04 | 3.92E−06 | 2.29E−02 | 2.12E−04 | 0.022074 | 0.998193 |
| SNK:BSA | 50 | 5.50E−04 | 3.92E−06 | 2.29E−02 | 2.12E−04 | 0.022074 | 0.998193 |
| SNK:BSA | 25 | 5.50E−04 | 3.92E−06 | 2.29E−02 | 2.12E−04 | 0.022074 | 0.998193 |
| SNK:BSA | 12.5 | 5.50E−04 | 3.92E−06 | 2.29E−02 | 2.12E−04 | 0.022074 | 0.998193 |
| SNK:BSA | 6.25 | 5.50E−04 | 3.92E−06 | 2.29E−02 | 2.12E−04 | 0.022074 | 0.998193 |
| SNK:BSA | 3.13 | 5.50E−04 | 3.92E−06 | 2.29E−02 | 2.12E−04 | 0.022074 | 0.998193 |
| SNK:BSA | 1.56 | 5.50E−04 | 3.92E−06 | 2.29E−02 | 2.12E−04 | 0.022074 | 0.998193 |

TABLE 6
Affinity analysis between rehu5E3-IgG4 (40 ug/mL) and
cSNK:BSA standards (using AHC sensors)

| Sample | Conc. | | | Kon | | kon2 | kon2 |
|---|---|---|---|---|---|---|---|
| D ID | (nM) | KD1 (M) | KD2 (M) | (1/Ms) | kon Error | (1/Ms) | error |
| SNK:BSA | 100 | 5.57E−10 | 3.94E−09 | 6.92E+05 | 1.42E+04 | 4.44E+06 | 1.26E+05 |
| SNK:BSA | 50 | 5.57E−10 | 3.94E−09 | 6.92E+05 | 1.42E+04 | 4.44E+06 | 1.26E+05 |
| SNK:BSA | 25 | 5.57E−10 | 3.94E−09 | 6.92E+05 | 1.42E+04 | 4.44E+06 | 1.26E+05 |
| SNK:BSA | 12.5 | 5.57E−10 | 3.94E−09 | 6.92E+05 | 1.42E+04 | 4.44E+06 | 1.26E+05 |
| SNK:BSA | 6.25 | 5.57E−10 | 3.94E−09 | 6.92E+05 | 1.42E+04 | 4.44E+06 | 1.26E+05 |
| SNK:BSA | 3.13 | 5.57E−10 | 3.94E−09 | 6.92E+05 | 1.42E+04 | 4.44E+06 | 1.26E+05 |
| SNK:BSA | 1.56 | 5.57E−10 | 3.94E−09 | 6.92E+05 | 1.42E+04 | 4.44E+06 | 1.26E+05 |
| Sample | Conc. | Kdis | kdis | kdis2 | kdis2 | | |
| D ID | (nM) | (1/s) | Error | (1/s) | error | Full X\hat{ }2 | Full R\hat{ }2 |
| SNK:BSA | 100 | 3.86E−04 | 1.01E−05 | 1.75E−02 | 2.18E−04 | 0.022533 | 0.993115 |
| SNK:BSA | 50 | 3.86E−04 | 1.01E−05 | 1.75E−02 | 2.18E−04 | 0.022533 | 0.993115 |
| SNK:BSA | 25 | 3.86E−04 | 1.01E−05 | 1.75E−02 | 2.18E−04 | 0.022533 | 0.993115 |
| SNK:BSA | 12.5 | 3.86E−04 | 1.01E−05 | 1.75E−02 | 2.18E−04 | 0.022533 | 0.993115 |
| SNK:BSA | 6.25 | 3.86E−04 | 1.01E−05 | 1.75E−02 | 2.18E−04 | 0.022533 | 0.993115 |
| SNK:BSA | 3.13 | 3.86E−04 | 1.01E−05 | 1.75E−02 | 2.18E−04 | 0.022533 | 0.993115 |
| SNK:BSA | 1.56 | 3.86E−04 | 1.01E−05 | 1.75E−02 | 2.18E−04 | 0.022533 | 0.993115 |

Table 7 summarizes the affinity constants of humanized 5E3 IgG1, IgG2 and IgG4 constructs. The KD ranges were determined using 2:1 kinetics of binding of analyte (BSA-cSNK) in solution to two different binding sites on the surface (immobilized bivalent, monospecific 5E3) in comparison to murine 5E3 using biolayer interferometry (Octet RED96).

TABLE 7
Summary of Affinity Data

KD range (M)

| mAb | KD1 | KD2 |
|---|---|---|
| m5E3 | 3.30 × 10⁻¹⁰ | 1.59 × 10⁻¹⁰ |
| hu5E3 (IgG1) | 4.63 × 10⁻¹⁰ | 1.82 × 10⁻¹⁰ |
| hu5E3 (IgG2) | 1.18 × 10⁻⁹ | 6.39 × 10⁻⁹ |
| rehu5E3 (IgG2) | 8.14 × 10⁻⁹ | 1.28 × 10⁻⁹ |
| hu5E3 (IgG4) | 6.33 × 10⁻¹⁰ | 3.04 × 10⁻¹⁰ |
| rehu5E3 (IgG4) | 5.57 × 10⁻¹⁰ | 3.94 × 10⁻¹⁰ |

### Example 7

### ELISA Assay of Humanized 5E3 Monoclonal Antibodies

An ELISA was used to test the binding of the mAbs against cSNK-BSA peptide. The ELISA plate was coated with antigen (Ag), 1 μg/well of cSNK-BSA. The wells were blocked with 5% skim milk then probed with serially diluted 5E3 mAbs (0.01 μg/mL to 5 μg/mL) and binding was detected with a commercial goat anti-human HRP conjugate antibody. Positive, murine 5E3, and negative, BSA alone, controls were also run. The plate was read at 450 nm.

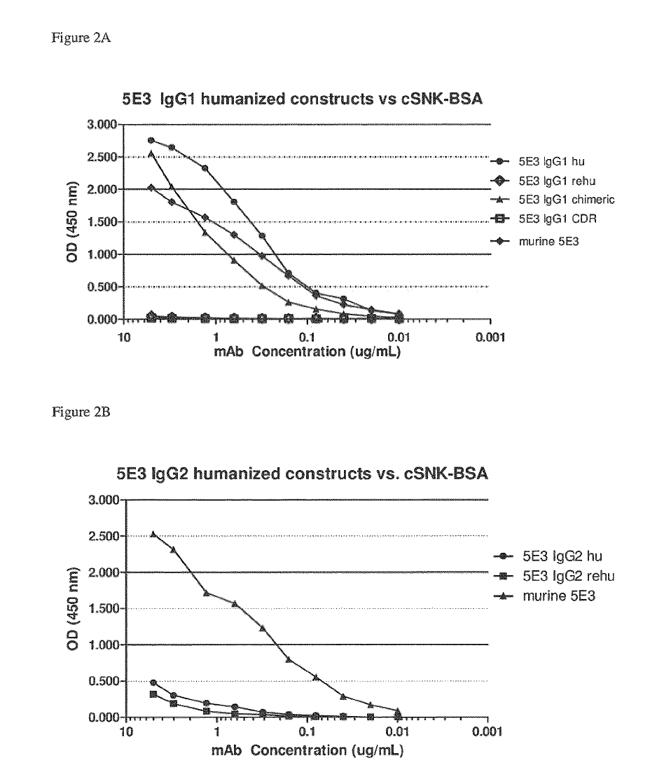

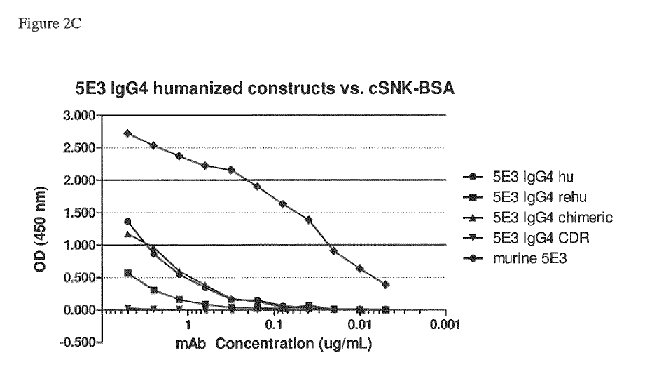

As shown in FIG. 2A, the hu5E3 IgG1 and chimeric IgG1 constructs demonstrated superior binding to cSNK-BSA compared to the murine 5E3. FIG. 2B shows results for the hu5E3 and rehu5E3 IgG2 constructs binding to cSNK-BSA compared to the murine 5E3. FIG. 2C shows results for all of the IgG4 humanized constructs tested. The IgG4 chimeric and hu versions show superior binding to the IgG4 rehu version, where the CDR version showed little binding. The differences between biolayer interferometry and direct ELISA results reflect the differences between the two different methodologies (vis-à-vis display and access of the cSNK epitope in a dynamic capacity or statically bound to the ELISA plate).

### Example 8

### Western Blot Analysis of Humanized 5E3 Monoclonal Antibodies

A 4-12% gradient SDS-PAGE gel was run with cSNK-BSA. The gel was then transferred to a nitrocellulose membrane. The next day the mAbs (1° Ab) were diluted to 2 μg/ml to 5 μg/ml depending on the antibody, and used to probe the membrane at room temperature (RT). The membranes were then probed with anti-mouse IgG-HRP (2° Ab).

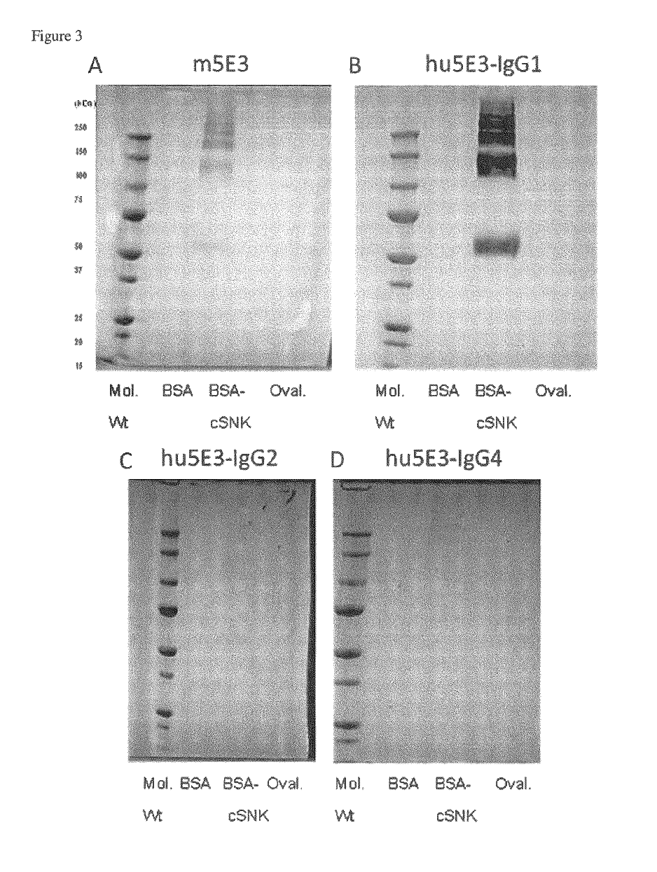

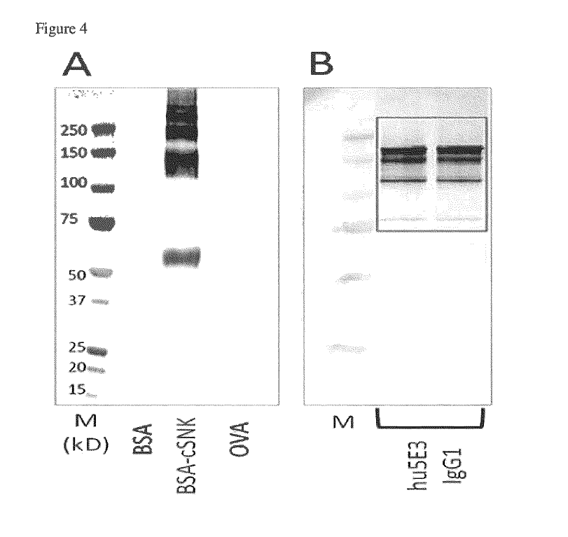

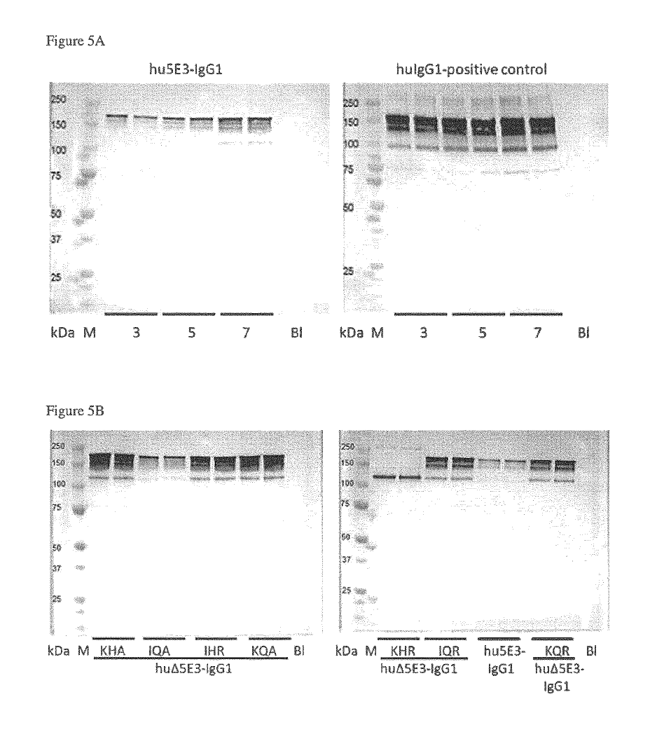

Results: As shown in FIG. 3A-D, humanized versions of 5E3 mAb showed binding to cSNK-BSA. As shown in FIGS. 4A and 4B, hu5E3 IgG1 binds to BSA-cSNK and is confirmed to be IgG1.

### Example 9

### Affinity Maturation

In silico modeling was used to increase the affinity of hu5E3 IgG1 derived antibodies to a cSNK peptide described herein. Antibodies to small peptides like SNK generally use two to three CDRs and typically form a deeper groove than the topography of a protein-protein interaction. Typically HCDR3 is the main interacting loop as it has the highest potential for mutation due to V-D-J rearrangement. Using Discovery Studio software, a 3D structural model of hu5E3 antibody was used to identify heavy and light CDR3 residues that interact with the SNK peptide. A modeled surface layer algorithm was also used to identify the theoretical paratope groove. Using this information, CDR3 residues were mutated to residues with similar charge, size, and polarity. The conserved targeted mutations attempt to modify the paratope groove to increase electrostatic interactions and increase affinity of 5E3 to the SNK peptide.

Primers were designed (Table 8) and site directed mutagenesis was performed on the hu5E3 vector.

TABLE 8
Site Directed Mutagenesis Primers Used to Generate Affinity
Maturation Constructs.

| SEQ ID | | | |
|---|---|---|---|
| NO. | Nickname | | |
| 222 | 5R48D-AFF | PRIMER | GTGACAATCACTTGTgacGCTTCCCAGGAAATT |
| 223 | 3R48D-AFF | PRIMER | AATTTCCTGGGAAGCgtcACAAGTGATTGTCAC |
| 224 | 5S50D-AFF | PRIMER | ATCACTTGTCGCGCTgacCAGGAAATTAGCGGA |
| 225 | 3S50D-AFF | PRIMER | TCCGCTAATTTCCTGgtcAGCGCGACAAGTGAT |
| 226 | 5Q51D-AFF | PRIMER | ACTTGTCGCGCTTCCgacGAAATTAGCGGATAC |
| 227 | 3Q51D-AFF | PRIMER | GTATCCGCTAATTTCgtcGGAAGCGCGACAAGT |
| 228 | 5E52D-AFF | PRIMER | TGTCGCGCTTCCCAGgacATTAGCGGATACCTG |
| 229 | 3E52D-AFF | PRIMER | CAGGTATCCGCTAATgtcCTGGGAAGCGCGACA |
| 230 | 5S54D-AFF | PRIMER | GCTTCCCAGGAAATTgacGGATACCTGACTTGG |
| 231 | 3S54D-AFF | PRIMER | CCAAGTCAGGTATCCgtcAATTTCCTGGGAAGC |
| 232 | 5A74D-AFF | PRIMER | AAGCGACTGATCTACgacGCATCTACCCTGGAC |
| 233 | 3A74D-AFF | PRIMER | GTCCAGGGTAGATGCgtcGTAGATCAGTCGCTT |
| 234 | 5S76D-AFF | PRIMER | CTGATCTACGCTGCAgacACCCTGGACAGTGGA |
| 235 | 3S76D-AFF | PRIMER | TCCACTGTCCAGGGTgtcTGCAGCGTAGATCAG |
| 236 | 5T77D-AFF | PRIMER | ATCTACGCTGCATCTgacCTGGACAGTGGAGTG |
| 237 | 3T77D-AFF | PRIMER | CACTCCACTGTCCAGgtcAGATGCAGCGTAGAT |
| 238 | 5L78D-AFF | PRIMER | TACGCTGCATCTACCgacGACAGTGGAGTGCCT |
| 239 | 3L78D-AFF | PRIMER | AGGCACTCCACTGTCgtcGGTAGATGCAGCGTA |
| 240 | 5S80D-AFF | PRIMER | GCATCTACCCTGGACgacGGAGTGCCTAAGAGG |
| 241 | 3S80D-AFF | PRIMER | CCTCTTAGGCACTCCgtcGTCCAGGGTAGATGC |
| 242 | 5G81D-AFF | PRIMER | TCTACCCTGGACAGTgacGTGCCTAAGAGGTTC |
| 243 | 3G81D-AFF | PRIMER | GAACCTCTTAGGCACgtcACTGTCCAGGGTAGA |
| 244 | 5A116D-AFF | PRIMER | AACTGCCTGCAGTACgacAATTATCCTAGAACA |
| 245 | 3A116D-AFF | PRIMER | TGTTCTAGGATAATTgtcGTACTGCAGGCAGTT |
| 246 | 5N117D-AFF | PRIMER | TGCCTGCAGTACGCCgacTATCCTAGAACATTT |
| 247 | 3N117D-AFF | PRIMER | AAATGTTCTAGGATAgtcGGCGTACTGCAGGCA |
| 248 | 5T183D-AFF | PRIMER | TCCGGTTATATCTTCgacTCCTACTATATCCAG |
| 249 | 3T183D-AFF | PRIMER | CTGGATATAGTAGGAgtcGAAGATATAACCGGA |
| 250 | 5S184D-AFF | PRIMER | GGTTATATCTTCACCgacTACTATATCCAGTGG |
| 251 | 3S184D-AFF | PRIMER | CCACTGGATATAGTAgtcGGTGAAGATATAACC |
| 252 | 5G207D-AFF | PRIMER | GGATGGATCTACCCTgacAACGTGAATACAAAG |
| 253 | 3G207D-AFF | PRIMER | CTTTGTATTCACGTTgtcAGGGTAGATCCATCC |
| 254 | 5N208D-AFF | PRIMER | TGGATCTACCCTGGGgacGTGAATACAAAGTAT |
| 255 | 3N208D-AFF | PRIMER | ATACTTTGTATTCACgtcCCCAGGGTAGATCCA |
| 256 | 5V209D-AFF | PRIMER | ATCTACCCTGGGAACgacAATACAAAGTATAAC |
| 257 | 3V209D-AFF | PRIMER | GTTATACTTTGTATTgtcGTTCCCAGGGTAGAT |
| 258 | 5N210D-AFF | PRIMER | TACCCTGGGAACGTGgacACAAAGTATAACGAG |
| 259 | 3N210D-AFF | PRIMER | CTCGTTATACTTTGTgtcCACGTTCCCAGGGTA |
| 260 | 5T211D-AFF | PRIMER | CCTGGGAACGTGAATgacAAGTATAACGAGAAG |
| 261 | 3T211D-AFF | PRIMER | CTTCTCGTTATACTTgtcATTCACGTTCCCAGG |
| 262 | 5K212D-AFF | PRIMER | GGGAACGTGAATACAgacTATAACGAGAAGTTC |
| 263 | 3K212D-AFF | PRIMER | GAACTTCTCGTTATAgtcTGTATTCACGTTCCC |
| 264 | 5E255D-AFF | PRIMER | GCTAGAATGGATTACgacGCCCACTATTGGGGA |
| 265 | 3E255D-AFF | PRIMER | TCCCCAATAGTGGGCgtcGTAATCCATTCTAGC |
| 266 | 5R48Y-AFF | PRIMER | GTGACAATCACTIGTtacGCTICCCAGGAAATT |
| 267 | 3R48Y-AFF | PRIMER | AATTTCCTGGGAAGCgtaACAAGTGATTGTCAC |
| 268 | 5S50Y-AFF | PRIMER | ATCACTIGTCGCGCTtacCAGGAAATTAGCGGA |
| 269 | 3S50Y-AFF | PRIMER | TCCGCTAATTTCCTGgtaAGCGCGACAAGTGAT |
| 270 | 5Q51Y-AFF | PRIMER | ACTTGTCGCGCTTCCtacGAAATTAGCGGATAC |
| 271 | 3Q51Y-AFF | PRIMER | GTATCCGCTAATTTCgtaGGAAGCGCGACAAGT |
| 272 | 5E52Y-AFF | PRIMER | TGTCGCGCTTCCCAGtacATTAGCGGATACCTG |
| 273 | 3E52Y-AFF | PRIMER | CAGGTATCCGCTAATgtaCTGGGAAGCGCGACA |
| 274 | 5S54Y-AFF | PRIMER | GCTICCCAGGAAATTtacGGATACCTGACTIGG |
| 275 | 3S54Y-AFF | PRIMER | CCAAGTCAGGTATCCgtaAATTTCCTGGGAAGC |
| 276 | 5A74Y-AFF | PRIMER | AAGCGACTGATCTACtacGCATCTACCCTGGAC |
| 277 | 3A74Y-AFF | PRIMER | GTCCAGGGTAGATGCgtaGTAGATCAGTCGCTT |
| 278 | 5S76Y-AFF | PRIMER | CTGATCTACGCTGCAtacACCCIGGACAGIGGA |
| 279 | 3S76Y-AFF | PRIMER | TCCACTGTCCAGGGTgtaTGCAGCGTAGATCAG |
| 280 | 5T77Y-AFF | PRIMER | ATCTACGCTGCATCTtacCIGGACAGIGGAGTG |
| 281 | 3T77Y-AFF | PRIMER | CACTCCACTGTCCAGgtaAGATGCAGCGTAGAT |
| 282 | 5L78Y-AFF | PRIMER | TACGCTGCATCTACCtacGACAGTGGAGTGCCT |
| 283 | 3L78Y-AFF | PRIMER | AGGCACTCCACTGTCgtaGGTAGATGCAGCGTA |
| 284 | 5S80Y-AFF | PRIMER | GCATCTACCCTGGACtacGGAGTGCCTAAGAGG |
| 285 | 3S80Y-AFF | PRIMER | CCTCTTAGGCACTCCgtaGTCCAGGGTAGATGC |
| 286 | 5G81Y-AFF | PRIMER | TCTACCCIGGACAGTtacGTGCCTAAGAGGITC |
| 287 | 3G81Y-AFF | PRIMER | GAACCTCTTAGGCACgtaACTGTCCAGGGTAGA |
| 288 | 5A116Y-AFF | PRIMER | AACTGCCTGCAGTACtacAATTATCCTAGAACA |
| 289 | 3A116Y-AFF | PRIMER | TGTTCTAGGATAATTgtaGTACTGCAGGCAGTT |
| 290 | 5N117Y-AFF | PRIMER | TGCCTGCAGTACGCCtacTATCCTAGAACATTT |
| 291 | 3N117Y-AFF | PRIMER | AAATGTTCTAGGATAgtaGGCGTACTGCAGGCA |
| 292 | 5T183Y-AFF | PRIMER | TCCGGTTATATCTTCtacTCCTACTATATCCAG |
| 293 | 3T183Y-AFF | PRIMER | CTGGATATAGTAGGAgtaGAAGATATAACCGGA |
| 294 | 5S184Y-AFF | PRIMER | GGTTATATCTTCACCtacTACTATATCCAGTGG |
| 295 | 3S184Y-AFF | PRIMER | CCACTGGATATAGTAgtaGGTGAAGATATAACC |
| 296 | 5G207Y-AFF | PRIMER | GGATGGATCTACCCItacAACGTGAATACAAAG |
| 297 | 3G207Y-AFF | PRIMER | CTTTGTATTCACGTTgtaAGGGTAGATCCATCC |
| 298 | 5N208Y-AFF | PRIMER | TGGATCTACCCTGGGtacGTGAATACAAAGTAT |
| 299 | 3N208Y-AFF | PRIMER | ATACTTTGTATTCACgtaCCCAGGGTAGATCCA |
| 300 | 5V209Y-AFF | PRIMER | ATCTACCCTGGGAACtacAATACAAAGTATAAC |
| 301 | 3V209Y-AFF | PRIMER | GTTATACTTTGTATTgtaGTTCCCAGGGTAGAT |
| 302 | 5N210Y-AFF | PRIMER | TACCCTGGGAACGTGtacACAAAGTATAACGAG |
| 303 | 3N210Y-AFF | PRIMER | CTCGTTATACTTTGTgtaCACGTTCCCAGGGTA |
| 304 | 5T211Y-AFF | PRIMER | CCIGGGAACGTGAATtacAAGTATAACGAGAAG |
| 305 | 3T211Y-AFF | PRIMER | CTTCTCGTTATACTTgtaATTCACGTTCCCAGG |
| 306 | 5K212Y-AFF | PRIMER | GGGAACGTGAATACAtacTATAACGAGAAGTTC |
| 307 | 3K212Y-AFF | PRIMER | GAACTTCTCGTTATAgtaTGTATTCACGTTCCC |
| 308 | 5E255Y-AFF | PRIMER | GCTAGAATGGATTACtacGCCCACTATTGGGGA |
| 309 | 3E255Y-AFF | PRIMER | TCCCCAATAGTGGGCgtaGTAATCCATTCTAGC |

Individual mutations were screened for binding to SNK-BSA and for loss or increase of affinity using the Octet Red (Table 9). Mutations in the light and heavy chains that increased affinity were then combined and rescreened.

TABLE 9
Screening for Loss or Increase of Binding of Affinity Maturation Constructs.

| | | | | Characterization compared to hu5E3 (Octet) | | |
|---|---|---|---|---|---|---|
| | | | | Binding to | | |
| | | Amino acid | | cSNK:BSA | Affinity - KD1 | Affinity - KD2 |
| | Chain | Mutation | Control | (at 100 nM) | (M) | (M) |
| Single Maturation | | | | | | |
| Code | | | | | | |
| | | | | | 6.27E−09 | 1.51E−08 |
| 5E3HCDR3-M206K | Heavy | Lysine | Methionine | Decreased | Not tested | Not tested |
| 5E3HCDR3-M206I | Heavy | Isoleucine | Methionine | Decreased | Not tested | Not tested |
| 5E3HCDR3-D207E | Heavy | Glutamic acid | Aspartic acid | Decreased | Not tested | Not tested |
| 5E3HCDR3-D207S | Heavy | Serine | Aspartic acid | Decreased | Not tested | Not tested |
| 5E3HCDR3-D207N | Heavy | Asparagine | Aspartic acid | Decreased | Not tested | Not tested |
| 5E3HCDR3-Y208F | Heavy | Phenylalanine | Tyrosine | Comparable | 2.15E−08 | 1.0E−12 |
| 5E3HCDR3-E209Q | Heavy | Glutamine | Glutamic acid | Comparable | 1.86E−09 | 6.05E−09 |
| 5E3HCDR3-E209D | Heavy | Aspartic acid | Glutamic acid | Comparable | 3.21E−09 | 9.13E−09 |
| 5E3HCDR3-A210G | Heavy | Glycine | Alanine | Comparable | 1.73E−09 | 9.97E−09 |
| 5E3HCDR3-A210V | Heavy | Valine | Alanine | Comparable | Not tested | Not tested |
| 5E3HCDR3-H211F | Heavy | Phenylalanine | Histidine | Decreased | Not tested | Not tested |
| 5E3HCDR3-H211N | Heavy | Asparagine | Histidine | Decreased | Not tested | Not tested |
| 5E3HCDR3-Y212F | Heavy | Phenylalanine | Tyrosine | Comparable | 1.58E−09 | 8.25E−09 |
| 5E3HCDR1-T183D | Heavy | Aspartic acid | Threonine | Decreased | Not tested | Not tested |
| 5E3HCDR1-S184D | Heavy | Aspartic acid | Serine | Comparable | 2.14E−10 | 3.41E−09 |
| 5E3HCDR2-G207D | Heavy | Aspartic acid | Glycine | Decreased | Not tested | Not tested |
| 5E3HCDR2-N208D | Heavy | Aspartic acid | Asparagine | Decreased | Not tested | Not tested |
| 5E3HCDR2-V209D | Heavy | Aspartic acid | Valine | Decreased | Not tested | Not tested |
| 5E3HCDR2-N210D | Heavy | Aspartic acid | Asparagine | Decreased | Not tested | Not tested |
| 5E3HCDR2-T211D | Heavy | Aspartic acid | Threonine | Decreased | Not tested | Not tested |
| 5E3HCDR2-K212D | Heavy | Aspartic acid | Lysine | Comparable | 1.71E−11 | 2.36E−09 |
| 5E3HCDR3-E255D | Heavy | Aspartic acid | Glutaminc acid | Comparable | 3.33E−11 | 2.57E−09 |
| 5E3HCDR1-T183Y | Heavy | Tyrosine | Threonine | Decreased | Not tested | Not tested |
| 5E3HCDR1-S184Y | Heavy | Tyrosine | Serine | Decreased | Not tested | Not tested |
| 5E3HCDR2-G207Y | Heavy | Tyrosine | Glycine | Decreased | Not tested | Not tested |
| 5E3HCDR2-N208Y | Heavy | Tyrosine | Asparagine | Decreased | Not tested | Not tested |
| 5E3HCDR2-V209Y | Heavy | Tyrosine | Valine | Comparable | 1.07E−11 | 2.40E−09 |
| 5E3HCDR2-N210Y | Heavy | Tyrosine | Asparagine | Decreased | Not tested | Not tested |
| 5E3HCDR2-T211Y | Heavy | Tyrosine | Threonine | Decreased | Not tested | Not tested |
| 5E3HCDR2-K212Y | Heavy | Tyrosine | Lysine | Decreased | Not tested | Not tested |
| 5E3HCDR3-E255Y | Heavy | Tyrosine | Glutamic acid | Decreased | Not tested | Not tested |
| 5E3KCDR3-L89V | Kappa | Valine | Leucine | Decreased | Not tested | Not tested |
| 5E3KCDR3-L89I | Kappa | Isoleucine | Leucine | Comparable | 1.62E−09 | 1.74E−08 |
| 5E3KCDR3-Q90N | Kappa | Asparagine | Glutamine | Decreased | Not tested | Not tested |
| 5E3KCDR3-Q90E | Kappa | Glutamic acid | Glutamine | Did not bind | Not tested | Not tested |
| 5E3KCDR3-Y91F | Kappa | Phenylalanine | Tyrosine | Did not bind | Not tested | Not tested |
| 5E3KCDR3-N93Q | Kappa | Glutamine | Asparagine | Comparable | 9.12E−09 | 7.60E−10 |
| 5E3KCDR3-N93D | Kappa | Aspartic acid | Asparagine | Comparable | 4.25E−09 | 9.90E−09 |
| 5E3KCDR3-Y94F | Kappa | Phenylalanine | Tyrosine | Did not bind | Not tested | Not tested |
| 5E3KCDR1-R48D | Kappa | Aspartic acid | Arginine | Decreased | Not tested | Not tested |
| 5E3KCDR1-S50D | Kappa | Aspartic acid | Serine | Decreased | Not tested | Not tested |
| 5E3KCDR1-Q51D | Kappa | Aspartic acid | Glutamine | Decreased | Not tested | Not tested |
| 5E3KCDR1-E52D | Kappa | Aspartic acid | Glutamic acid | Decreased | Not tested | Not tested |
| 5E3KCDR1-S54D | Kappa | Aspartic acid | Serine | Decreased | Not tested | Not tested |
| 5E3KCDR2-A74D | Kappa | Aspartic acid | Alanine | Comparable | 3.02E−10 | 3.06E−09 |
| 5E3KCDR2-2S76D | Kappa | Aspartic acid | Serine | Decreased | Not tested | Not tested |
| 5E3KCDR2-2T77D | Kappa | Aspartic acid | Threonine | Decreased | Not tested | Not tested |
| 5E3KCDR2-L78D | Kappa | Aspartic acid | Leucine | Decreased | Not tested | Not tested |
| 5E3KCDR2-S80D | Kappa | Aspartic acid | Serine | Comparable | 2.69E−11 | 3.95E−09 |
| 5E3KCDR2-G81D | Kappa | Aspartic acid | Glycine | Comparable | 1.92E−11 | 2.33E−09 |
| 5E3KCDR3-A116D | Kappa | Aspartic acid | Alanine | Decreased | Not tested | Not tested |
| 5E3KCDR3-N117D | Kappa | Aspartic acid | Asparagine | Decreased | Not tested | Not tested |
| 5E3KCDR1-R48Y | Kappa | Tyrosine | Arginine | Decreased | Not tested | Not tested |
| 5E3KCDR1-S50Y | Kappa | Tyrosine | Serine | Decreased | Not tested | Not tested |
| 5E3KCDR1-Q51Y | Kappa | Tyrosine | Glutamine | Decreased | Not tested | Not tested |
| 5E3KCDR1-E52Y | Kappa | Tyrosine | Glutamic acid | Decreased | Not tested | Not tested |
| 5E3KCDR1-S54Y | Kappa | Tyrosine | Serine | Decreased | Not tested | Not tested |
| 5E3KCDR2-A74Y | Kappa | Tyrosine | Alanine | Decreased | Not tested | Not tested |
| 5E3KCDR2-S76Y | Kappa | Tyrosine | Serine | Decreased | Not tested | Not tested |
| 5E3KCDR2-T77Y | Kappa | Tyrosine | Threonine | Decreased | Not tested | Not tested |
| 5E3KCDR2-L78Y | Kappa | Tyrosine | Leucine | Decreased | Not tested | Not tested |
| 5E3KCDR2-S80Y | Kappa | Tyrosine | Serine | Comparable | 1.38E−11 | 3.05E−09 |
| 5E3KCDR2-G81Y | Kappa | Tyrosine | Glycine | Decreased | Not tested | Not tested |
| 5E3KCDR3-A116Y | Kappa | Tyrosine | Alanine | Comparable | <1.0E−12 | 1.93E−09 |
| 5E3KCDR3-N117Y | Kappa | Tyrosine | Asparagine | Decreased | Not tested | Not tested |
| Double | | | | | | |
| Combination | | | | | | |
| Code | | | | | | |
| (Heavy/Kappa | | | | | | |
| combination) | | | | | | |
| 5E3KCDR3-L89I | Kappa | Isoleucine | Leucine | Decreased | Not tested | Not tested |
| 5E3HCDR3-Y208F | Heavy | Phenylalanine | Tyrosine | | | |
| 5E3KCDR3-L89I | Kappa | Isoleucine | Leucine | Comparable | 1.98E−08 | — |
| 5E3HCDR3-E209Q | Heavy | Glutamine | Glutamic acid | | | |
| 5E3KCDR3-L89I | Kappa | Isoleucine | Leucine | Decreased | Not tested | Not tested |
| 5E3HCDR3-A210G | Heavy | Glycine | Alanine | | | |
| 5E3KCDR3-L89I | Kappa | Isoleucine | Leucine | Comparable | 2.10E−11 | 4.20E−09 |
| 5E3HCDR3-Y212F | Heavy | Phenylalanine | Tyrosine | | | |
| 5E3KCDR3-N93Q | Kappa | Glutamine | Asparagine | Decreased | Not tested | Not tested |
| 5E3HCDR3-Y208F | Heavy | Phenylalanine | Tyrosine | | | |
| 5E3KCDR3-N93Q | Kappa | Glutamine | Asparagine | Decreased | Not tested | Not tested |
| 5E3HCDR3-E209Q | Heavy | Glutamine | Glutamic acid | | | |
| 5E3KCDR3-N93Q | Kappa | Glutamine | Asparagine | Decreased | Not tested | Not tested |
| 5E3HCDR3-A210G | Heavy | Glycine | Alanine | | | |
| 5E3KCDR3-N93Q | Kappa | Glutamine | Asparagine | Comparable | 2.01E−11 | 2.80E−09 |
| 5E3HCDR3-Y212F | Heavy | Phenylalanine | Tyrosine | | | |
| 5E3KCDR3-N93D | Kappa | Aspartic acid | Asparagine | Decreased | Not tested | Not tested |
| 5E3HCDR3-Y208F | Heavy | Phenylalanine | Tyrosine | | | |
| 5E3KCDR3-N93D | Kappa | Aspartic acid | Asparagine | Decreased | Not tested | Not tested |
| 5E3HCDR3-E209Q | Heavy | Glutamine | Glutamic acid | | | |
| 5E3KCDR3-N93D | Kappa | Aspartic acid | Asparagine | Decreased | Not tested | Not tested |
| 5E3HCDR3-A210G | Heavy | Glycine | Alanine | | | |
| 5E3KCDR3-N93D | Kappa | Aspartic acid | Asparagine | Comparable | 1.94E−11 | 2.41E−09 |
| 5E3HCDR3-Y212F | Heavy | Phenylalanine | Tyrosine | | | |

### Example 11

### Enhanced Expression of Humanized 5E3 (Development of huΔ5E3)

Aside from improvements for affinity (e.g. affinity maturation) and biological activity (e.g. control pharmacodynamic behavior, prolonged half-life, increased bioavailability, etc), the engineering of the antibody was performed to increase expression levels in transiently tranfected mammalian cells to advance the most productive sequences into stable cell lines. For the scope of the present invention this example provides modifications that either increased expression levels of the humanized constructs expressing IgG1, IgG2 and/or IgG4, had a negative impact or no impact but does not limit further modifications to the sequence of either the heavy or light chains for similar purposes.

Different combinations of amino acid substitutions were designed within the heavy chain variable region as summarized in Table 10 which shows alignments of different amino acid substitutions performed to enhance overall expression in transiently transfected CHO cells for advancing and selection constructs for stable cell lines.

TABLE 10
Alignment of Different Amino Acid Substitutions Performed to Enhance
Overall Expression in Transiently Transfected CHO Cells.

| | | Effect on transient | Retained |
|---|---|---|---|
| Isotype | Construct | expression | affinity |
| IgG1 | Hu5E3-IgG1 | Baseline | Baseline |
| | HuΔ5E3-IgG1 (KHA) | ++++ | Yes |
| | HuΔ5E3-IgG1 (IHR) | ++++ | Yes |
| | HuΔ5E3-IgG1 (KQA) | ++++ | Yes |
| | HuΔ5E3-IgG1 (IQR) | ++++ | Yes |
| | HuΔ5E3-IgG1 (KQR) | ++++ | Yes |
| | HuΔ5E3-IgG1 (KHR) | HC only | N/A |
| | | (destabilized | |
| | | HC:LC interface) | |
| | HuΔ5E3-IgG1 (49A) | No effect | N/A |
| | HuΔ5E3-IgG1 (12V) | No effect | N/A |
| | HuΔ5E3-IgG1 (VAY) | No effect | N/A |
| | HuΔ5E3-IgG1 (VAW) | No effect | N/A |
| IgG2 | Hu5E3-IgG2 | Baseline | Baseline |
| | HuΔ5E3-IgG2 (KQR) | ++++ | Yes |
| IgG4 | Hu5E3-IgG4 | Baseline | Baseline |
| | HuΔ5E3-IgG4 (KQR) | ++++ | Yes |

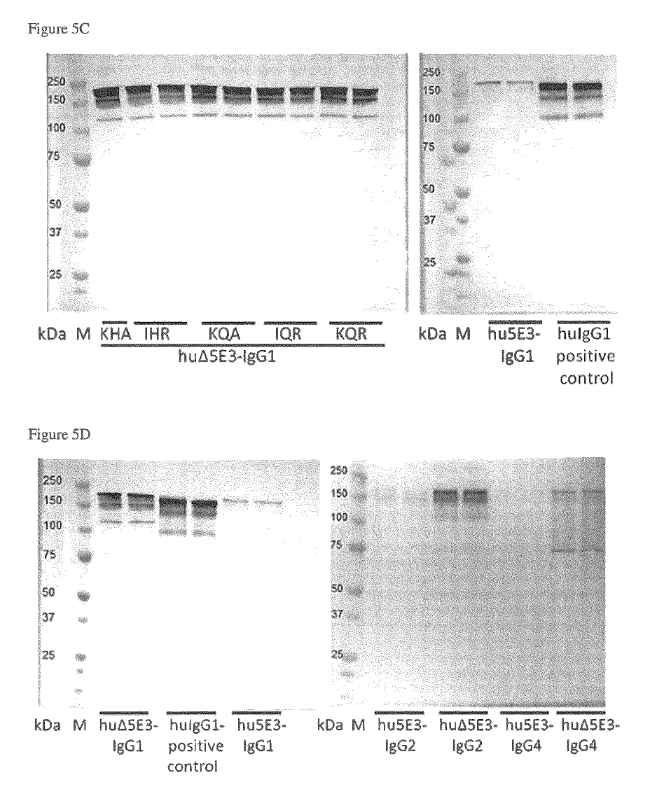

Each construct was used for transient expression analysis as presented in FIGS. 5A-D, from which qualitative effects on expression were observed (across normalized sampling). Amino acid changes were incorporated by site directed mutagenesis, utilizing complementary oligonucleotides in combination with GeneArt Site-Directed Mutagenesis Kits and AccuPrime Pfx DNA polymerase (Life Technologies). This system allows for base substitutions of up to 12 nucleotides (encoding for a stretch of 4 amino acids) on vectors upto 14 kb. Upon subcloning and confirmation of correct sequence changes by sequence analysis (SEQ ID NOs: 123-132), the vectors were used to transfect CHO-S and/or CHO-K1SV cell culture. For expression analysis, a baseline of expression was established over the first 7 days post-transfection using hu5E3-IgG1 and a human IgG1 positive control known to result in high level stable expression in CHO-K1SV (FIG. 5A). Once it was established that day 3 provided marked differences in relative expression between hu5E3-IgG1 and the positive control, CHO cells were transfected with the huΔ5E3 constructs and expression levels of mAb from supernatants analysed 3 days post transfection. Briefly, SDS-solubilized (nonreduced) cell culture supernatants normalized to a cell concentration of 1×10⁴ cells per well were separated by SDS-PAGE and blotted to nitrocellulose. Blots were then probed with Goat anti-human IgG-HRP and developed. Expression levels were then compared to the benchmark hu5E3-IgG1 and/or positive control (huIgG1) to calibrate. Comparisons between the huΔ5E3-IgG1 variants are shown in FIG. 5B and FIG. 5C, while huΔ5E3-IgG2 KQR and huΔ5E3-IgG4 KQR are shown in FIG. 5D. From this analysis, some amino acid substitutions in framework 2 clearly enhanced expression levels in comparison to the first generation humanized IgG1, IgG2 and IgG4 constructs.

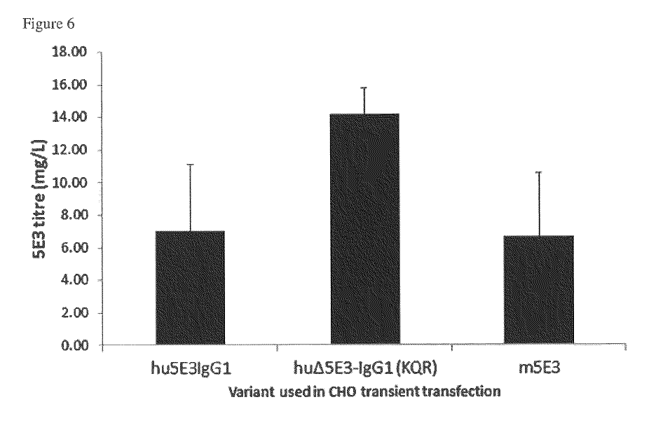

FIG. 6 shows expression titer from transient expression in CHO cells. m5E3 represents the transient expression of the murine 5E3 antibody. Overall, effects on transient expression levels were enhanced in relation to the original hu5E-IgG1 and murine 5E3, while affinity was retained as demonstrated in Example 12.

### Example 12

### Characterization of Humanized Δ5E3 mAbs

ELISA Assay of Humanized 5E3 and Δ5E3 Monoclonal Antibodies

An ELISA was used to confirm the Δ5E3 mAb had retained immunorecognition of the cyclic SNK epitope. The ELISA plate was coated with antigen (Ag) (100 μl at 1 μg/mL Ag/well) of either cSNK-BSA (head-to-tail cyclized peptide of SEQ ID NO: 3) or ccSNK-BSA (head-to-tail cyclized peptide of SEQ ID NO: 8). The wells were blocked with 5% skim milk then probed with serially diluted 5E3 mAbs (0.001 μg/mL to 1 μg/mL) and binding was detected with a commercial goat anti-human HRP conjugate antibody. Positive, murine 5E3, and negative, BSA alone, controls were also run. The plate was read at 450 nm.

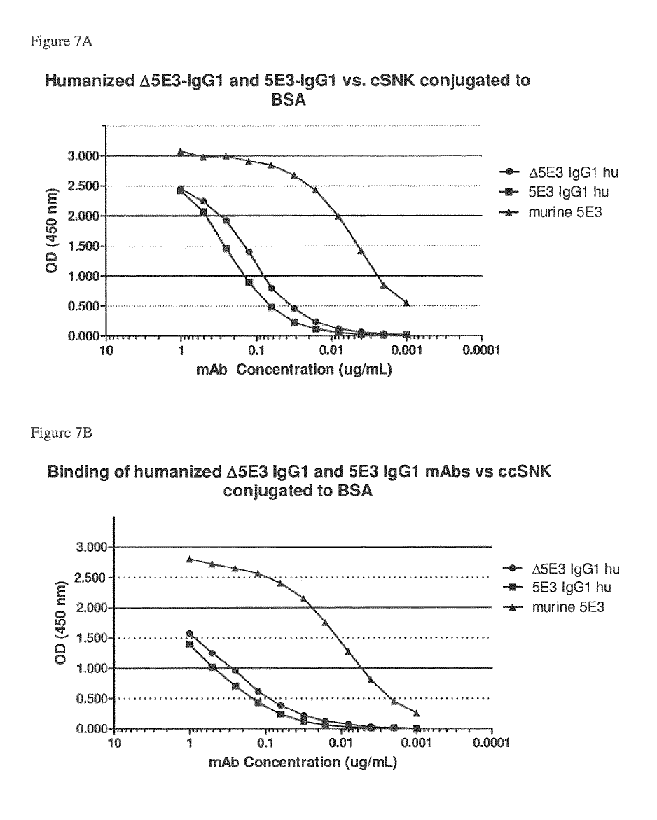

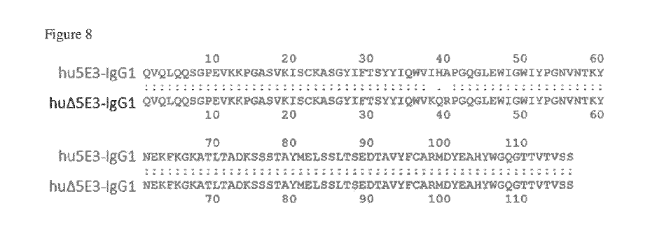

As shown in FIGS. 7A and 7B, both the hu5E3-IgG1 (hu5E3) and huΔ5E3-IgG1 (hu5E3 IgG1 KQR) demonstrated binding to cSNK-BSA and ccSNK-BSA, respectively, compared to the murine 5E3 (m5E3). FIG. 8 shows an alignment of heavy chain variable regions of hu5E3-IgG1 and huΔ5E3-IgG1 (KQR variant in framework 2) variant.

cSNK Affinity of Humanized 5E3 and Δ5E3 Monoclonal Antibodies

Biolayer interferometry (Octet Red92, ForteBio) was used to qualitatively/quantitatively assess the interactions between humanized 5E3 and Δ5E3 mAbs to the cSNK:BSA conjugate. To analyze affinity, sensors were washed in PBST (PBS+0.1% triton) until a stable baseline was achieved. MAbs were loaded onto sensors and then washed in PBST. Each sensor was associated with one of several cSNK:BSA standards (100 nM to 0 nM) and then washed in PBST. Data was processed statistically using ForteBio Data Analysis software to determine the strength of antigen-antibody binding (KD). For each mAb analyzed, a 1:1 model provided the “best fit”.

Results are summarized in Table 11. For each of the variants tested (i.e. first generation hu5E3-IgG1, IgG2 and IgG4 compared to huΔ5E3-IgG1, IgG2 and IgG4), the affinity towards the cSNK epitope was retained indicating that the amino acid substitutions incorporated into the HCVRs increased expression without impacting the binding to the cSNK epitope.

TABLE 11
Affinity of hu5E3 Mutation Variants to BSA-cSNK.

| | Variant (FR2 | KD range (M) |
|---|---|---|
| mAb | substitutions) | KD1 |
| hu5E3-IgG1 | IHA | 2.01 × 10⁻⁹ |
| huΔ5E3-IgG1 | KHA | 1.52 × 10⁻⁹ |
| | IHR | 1.43 × 10⁻⁹ |
| | KQA | 1.28 × 10⁻⁹ |
| | IQR | 1.26 × 10⁻⁹ |
| | KQR | 1.88 × 10⁻⁹ |
| hu5E3-IgG2 | IHA | 3.26 × 10⁻¹⁰ |
| huΔ5E3-IgG2 | KQR | 2.26 × 10⁻¹⁰ |
| hu5E3-IgG4 | IHA | 1.31 × 10⁻⁹ |
| huΔ5E3-IgG4 | KQR | 1.04 × 10⁻⁹ |

### Example 13

### Humanized 5E3 mAbs Bind Aβ Oligomers

Using a Biacore™ 3000, Aβ42 oligomers were immobilized (containing monomers, low and high molecular weight oligomers) and BSA (reference surface) on 4 separate flow cells of a biosensorchip. Murine 5E3 or humanized 5E3 IgG1, IgG2, and IgG4 were then diluted, sequentially injected over the different immobilized peptides and antibody binding monitored in real time. Binding data was collected by sequentially injecting buffer blanks, 1/10 dilutions of m5E3 or 1 uM dilutions of hu5E3 control over separate flow cells immobilized with BSA or Aβ42 oligomer on another biosensor chip at a flow rate of 10 μl/minute. Association was allowed for 200 seconds, followed by dissociation for 500 seconds and binding responses collected 10 seconds before the end of the dissociation. All binding responses were double-referenced by subtracting buffer blanks and BSA reference surface binding.

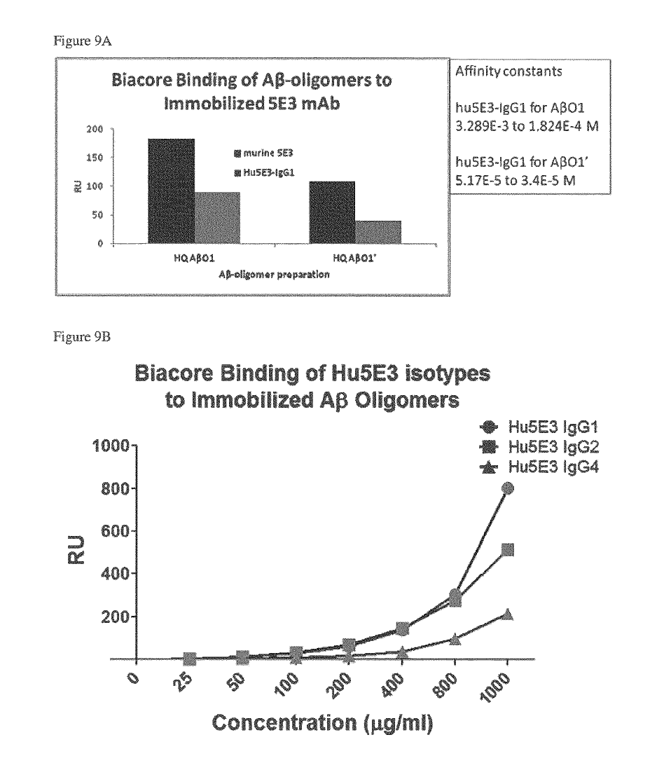

Results: As shown in FIG. 9A and FIG. 9B, the humanized 5E3 isotypes bound to the Aβ42 oligomer preparation. Affinity constants were determined for hu5E3 IgG1 Aβ42 oligomer preparations.

TABLE 12
SEQUENCES

| SEQ ID | | Chain, | |
|---|---|---|---|
| NO | Name | Region | Sequence |
| 1 | SNK peptide | | SNK |
| 2 | Aβ peptide #1 | | GSNKG |
| 3 | Aβ peptide #2 | | CGSNKGG |
| 4 | Aβ peptide #3 | | GSNK |
| 5 | Aβ peptide #4 | | SNKG |
| 6 | Aβ peptide #5 | | CSNKG |
| 7 | Aβ peptide #6 | | CGSNKGC |
| 8 | Aβ peptide #7 | | CCGSNKGC |
| 9 | Aβ peptide #8 | | GGSNKGC |
| 10 | 5E3 VL | VL | GACATCCAGATGACCCAGTCTCCATCCTCCTTATCTGCCTCTCTGGG |
| | | | AGAAAGAGTCAGTCTCACTTGTCGGGCAAGTCAGGAAATTAGTGGT |
| | | | TACTTAACCTGGCTTCAGCAGAAACCAGATGGAACTATTAAACGCCT |
| | | | GATCTACGCCGCATCCACTTTAGATTCTGGTGTCCCAAAAAGGTTCA |
| | | | GTGGCAGTAGGTCTGGGTCAGATTATTCTCTCACCATCAGCAGCCTT |
| | | | GAGTCTGAAGATTTTGCAGACTATAACTGTCTACAATATGCTAATTA |
| | | | TCCTCGGACGTTCGGTGGAGGCACCAAGCTGGAAATCAAA |
| 11 | 5E3 VL | VL | DIQMTQSPSSLSASLGERVSLTCRASQEISGYLTWLQQKPDGTIKRLIYA |
| | | | ASTLDSGVPKRFSGSRSGSDYSLTISSLESEDFADYNCLQYANYPRTFGG |
| | | | GTKLEIK |
| 12 | 5E3 LCDR1 | LCDR1 | QEISGY |
| 13 | 5E3 LCDR2 | LCDR2 | AASTLDSG |
| 14 | 5E3 LCDR3 | LCDR3 | LQYANYPRT |
| 15 | 5E3 VH | VH | CAGGTCCAGCTGCAGCAGTCTGGACCTGAGCTGGTGAAGCCTGGG |
| | | | GCTTCAGTGAGGATATCCTGCAAGGCTTCTGGCTACATATTCACAAG |
| | | | CTACTATATACAGTGGGTGATACACAGGCCTGGACAGGGACTTGAG |
| | | | TGGATTGGATGGATTTATCCTGGAAATGTTAATACTAAGTACAATGA |
| | | | GAAGTTCAAGGGCAAGGCCACACTGACTGCAGACAAATCCTCCAGC |
| | | | ACAGCCTACATGCAGCTCAGCAGATTGACCTCTGAGGACTCTGCGG |
| | | | TCTATTTCTGTGCAAGGATGGATTACGAGGCTCACTACTGGGGCCA |
| | | | AGGCACCACTCTCACAGTCTCCTCA |
| 16 | 5E3 VH | VH | QVQLQQSGPELVKPGASVRISCKASGYIFTSYYIQWVIHRPGQGLEWIG |
| | | | WIYPGNVNTKYNEKFKGKATLTADKSSSTAYMQLSRLTSEDSAVYFCAR |
| | | | MDYEAHYWGQGTTLTVSS |
| 17 | 5E3 HCDR1 | HCDR1 | GYIFTSYY |
| 18 | 5E3 HCDR2 | HCDR2 | IYPGNVNT |
| 19 | 5E3 HCDR3 | HCDR3 | ARMDYEAHY |
| 20 | CDR var | HCDR1 | ggatatacattcacctcttactat |
| 21 | CDR var | HCDR1 | GYTFTSYY |
| 22 | HuHFW1 | HFW1 | QVQLQQSGPEVKKPGASVKISCKAS |
| 23 | CDRHFW1 | HFW1 | QVQLVQSGAEVKKPGASVINSCKAS |
| 24 | HuHFW1 | HFW1 | QVQLQQSGPEVVKPGASVKISCKAS |
| | (also ″12V″) | | |
| 25 | HuHFW2/ReHu | HFW2 | atccagtgggtcatccacgcacctggtcagggactggaatggatcggatgg |
| | HFW2 | | |
| 26 | HuHFW2/ReHu | HFW2 | IQWVIHAPGQGLEWIGW |
| | HFW2 | | |
| 27 | CDRHFW2 | HFW2 | IQWVRQAPGQRLEWMGW |
| 28 | HuIHR | HFW2 | atccagtgggtcatccacaggcctggtcagggactggaatggatcggatgg |
| 29 | HuIHR | HFW2 | IQWVIHRPGQGLEWIGW |
| 30 | HuKHA | HFW2 | atccagtgggtcaagcacgcacctggtcagggactggaatggatcggatgg |
| 31 | HuKHA | HFW2 | IQWVKHAPGQGLEWIGW |
| 32 | HuKQR | HFW2 | atccagtgggtcaagcagaggcctggtcagggactggaatggatcggatgg |
| 33 | HuKQR | HFW2 | IQWVKQRPGQGLEWIGW |
| 34 | HuIQR | HFW2 | atccagtgggtcatccagaggcctggtcagggactggaatggatcggatgg |
| 35 | HuIQR | HFW2 | IQWVIQRPGQGLEWIGW |
| 36 | HuKQA | HFW2 | atccagtgggtcaagcaggcacctggtcagggactggaatggatcggatgg |
| 37 | HuKQA | HFW2 | IQWVKQAPGQGLEWIGW |
| 38 | HuKHA | HFW2 | IQWVKHAPGQGLEWIGW |
| 39 | HuKQR | HFW2 | IQWVKQRPGQGLEWIGW |
| 40 | HuIQR | HFW2 | IQWVIQRPGQGLEWIGW |
| 41 | HuKHR | HFW2 | IQWVKHRPGQGLEWIGW |
| 42 | Hu49A | HFW2 | IQWVIHAPGQGLEWIAW |
| 43 | HuVAY | HFW2 | IQWVIHAPGQGLEWVAY |
| 44 | HuVAW | HFW2 | IQWVIHAPGQGLEWVAW |
| 45 | HuHFW3 | HFW3 | KYNEKFKGKATLTADKSSSTAYMELSSLTSEDTAVYFC |
| 46 | CDRHFW3 | HFW3 | KYNEKFKGRVTITRDTSASTAYMELSSLRSEDTAVYYC |
| 47 | ReHuHFW3 | HFW3 | KYNEKFKGKATLTADKSSSTAYMELSSLRSEDSAVYFC |
| 48 | HuHFW4 | HFW4 | WGQGTIVIVSS |
| 49 | CDRHFW4/ReH | HFW4 | WGQGTLVTVSS |
| | uHFW4 | | |
| 50 | HuLFW1/rehuL | LFW1 | DIQMTQSPSSLSASVGDRVTITCRAS |
| | FW1 | | |
| 51 | CDRLFW1 | LFW1 | DIQMTQSPSSLSASLGDRVILTCRAS |
| 52 | HuLFW2 | LFW2 | LTWLQQKPEGAIKRLIY |
| 53 | CDRLFW2 | LFW2 | LTWYQQKPEKAPKSLIY |
| 54 | ReHuLFW2 | LFW2 | LTWLQQKPEKAIKRLIY |
| 55 | HuLFW3 | LFW3 | VPKRFSGSRSGSDYSLTISSLQPEDFATYNC |
| 56 | CDRLFW3 | LFW3 | VPSRFSGSGSGTDFTLTISSLQPEDFATYYC |
| 57 | ReHuLFW3 | LFW3 | VPKRFSGSRSGSDYTLTISSLQPEDFADYNC |
| 58 | HuLFW4/ReHu | LFW4 | FGGGTKLEIK |
| | LFW4 | | |
| 59 | CDRLFW4 | LFW4 | FGGGTKLEIK |
| 60 | 5E3 CDR | VL | GACATTCAGATGACCCAGAGCCCTAGTTCACTGAGTGCCTCAGTCG |
| | | | GGGATCGAGTGACTATCACCTGTCGTGCTAGTCAGGAAATTTCAGG |
| | | | TTACCTGACCTGGTATCAGCAGAAGCCAGAGAAAGCCCCCAAGAGC |
| | | | CTGATCTACGCTGCATCCACCCTGGACAGCGGAGTGCCATCTCGATT |
| | | | CTCCGGAAGCGGGTCTGGTACAGACTTTACACTGACTATTTCCAGCC |
| | | | TGCAGCCAGAGGATTTCGCAACTTACTATTGCCTGCAGTACGCCAAC |
| | | | TATCCCAGAACCTTTGGCGGAGGGACAAAAGTGGAAATCAAG |
| 61 | 5E3 CDR | VL | DIQMTQSPSSLSASVGDRVTITCRASQEISGYLTANYQQKPEKAPKSLIYA |
| | | | ASTLDSGVPSRFSGSGSGTDFTLTISSLQPEDFATYYCLQYANYPRTFGG |
| | | | GTKVEIK |
| 62 | 5E3 CDR | VH | CAGGTGCAGCTGGTCCAGTCAGGCGCAGAAGTGAAAAAACCCGGA |
| | | | GCATCAGTCAAAGTCTCTTGTAAGGCTAGCGGATATACATTCACCTC |
| | | | TTACTATATCCAGTGGGTGAGACAGGCTCCAGGACAGCGCCTGGAA |
| | | | TGGATGGGCTGGATCTACCCCGGAAACGTCAATACAAAGTATAACG |
| | | | AGAAGTTCAAAGGAAGGGTGACTATCACCCGGGACACATCAGCATC |
| | | | CACTGCCTACATGGAGCTGTCCAGCCTGAGATCCGAAGACACTGCC |
| | | | GTGTACTATTGCGCTCGCATGGATTACGAAGCCCACTATTGGGGTC |
| | | | AGGGCACTCTGGTCACCGTGTCTAGT |
| 63 | 5E3 CDR | VH | QVQLVQSGAEVKKPGASVINSCKASGYTFTSYYIQWVRQAPGQRLEW |
| | | | MGWIYPGNVNTKYNEKFKGRVTITRDTSASTAYMELSSLRSEDTAVYY |
| | | | CARMDYEAHYWGQGTLVTVSS |
| 64 | 5E3 hu | VL | GACATTCAGATGACTCAGTCTCCCAGCTCTCTGTCAGCCTCCGTCGG |
| | | | CGATAGAGTGACAATCACTTGTCGCGCTTCCCAGGAAATTAGCGGA |
| | | | TACCTGACTTGGCTGCAGCAGAAACCCGAGGGGGCCATCAAGCGAC |
| | | | TGATCTACGCTGCATCTACCCTGGACAGTGGAGTGCCTAAGAGGTT |
| | | | CAGCGGTTCTCGGAGTGGCTCAGACTACTCTCTGACTATCAGTTCAC |
| | | | TGCAGCCCGAGGATTTCGCAACCTATAACTGCCTGCAGTACGCCAAT |
| | | | TATCCTAGAACATTTGGCGGAGGGACTAAACTGGAAATCAAG |
| 65 | 5E3 hu | VL | DIQMTQSPSSLSASVGDRVTITCRASQEISGYLTANLQQKPEGAIKRLIYA |
| | | | ASTLDSGVPKRFSGSRSGSDYSLTISSLQPEDFATYNCLQYANYPRTFGG |
| | | | GTKLEIK |
| 66 | 5E3 hu | VH | CAGGTCCAGCTGCAGCAGAGCGGTCCCGAGGTCAAAAAACCCGGC |
| | | | GCATCCGTGAAAATCAGTTGTAAAGCATCCGGTTATATCTTCACCTC |
| | | | CTACTATATCCAGTGGGTCATCCACGCACCTGGTCAGGGACTGGAA |
| | | | TGGATCGGATGGATCTACCCTGGGAACGTGAATACAAAGTATAACG |
| | | | AGAAGTTCAAAGGCAAGGCTACACTGACTGCAGACAAGTCCAGCTC |
| | | | TACTGCATACATGGAGCTGAGTTCACTGACTAGCGAAGACACCGCC |
| | | | GTGTATTTCTGCGCTAGAATGGATTACGAAGCCCACTATTGGGGAC |
| | | | AGGGGACCACAGTCACCGTGTCCTCC |
| 67 | 5E3 hu | VH | QVQLQQSGPEVKKPGASVKISCKASGYIFTSYYIQWVIHAPGQGLEWIG |
| | | | WIYPGNVNTKYNEKFKGKATLTADKSSSTAYMELSSLTSEDTAVYFCAR |
| | | | MDYEAHYWGQGTTVTVSS |
| 68 | 5E3 rehu | VL | GACATTCAGATGACACAGAGCCCAAGCTCTCTGTCAGCCTCCCTGG |
| | | | GCGACAGAGTGACTCTGACCTGTCGCGCTTCTCAGGAAATCAGTGG |
| | | | CTACCTGACATGGCTGCAGCAGAAACCCGAGAAGGCCATCAAAAGA |
| | | | CTGATCTACGCTGCATCAACTCTGGACTCCGGCGTGCCTAAGAGGTT |
| | | | CAGCGGTTCTCGGAGTGGCTCAGATTACACACTGACTATTAGTTCAC |
| | | | TGCAGCCCGAGGACTTCGCAGATTATAACTGCCTGCAGTACGCCAA |
| | | | TTATCCTCGAACATTTGGCGGAGGGACTAAGCTGGAAATCAAA |
| 69 | 5E3 rehu | VL | DIQMTQSPSSLSASLGDRVILTCRASQEISGYLTANLQQKPEKAIKRLIYA |
| | | | ASTLDSGVPKRFSGSRSGSDYTLTISSLQPEDFADYNCLQYANYPRTFGG |
| | | | GTKLEIK |
| 70 | 5E3 rehu | VH | CAGGTCCAGCTGCAGCAGAGCGGTCCCGAGGTCGTGAAACCCGGA |
| | | | GCATCTGTGAAAATCAGTTGTAAGGCCAGCGGATACATCTTTACCTC |
| | | | TTACTATATCCAGTGGGTCATCCACGCACCTGGTCAGGGACTGGAAT |
| | | | GGATCGGATGGATCTACCCTGGGAACGTGAATACCAAGTATAACGA |
| | | | GAAGTTCAAAGGCAAGGCTACTCTGACCGCAGACAAGTCCAGCTCT |
| | | | ACAGCATACATGGAGCTGAGTTCACTGAGGTCCGAAGACAGCGCCG |
| | | | TGTATTTCTGCGCTCGGATGGATTACGAAGCCCACTATTGGGGACA |
| | | | GGGGACTCTGGTCACCGTGTCCTCC |
| 71 | 5E3 rehu | VH | QVQLQQSGPEVVKPGASVKISCKASGYIFTSYYIQWVIHAPGQGLEWI |
| | | | GWIYPGNVNTKYNEKFKGKATLTADKSSSTAYMELSSLRSEDSAVYFCA |
| | | | RMDYEAHYWGQGTLVTVSS |
| 72 | Mouse 5E3 | VL | gacatccagatgacccagtctccatcctccttatctgcctctctgggagaaagagtcagtct |
| | Codon | | cacttgtcgggcaagtcaggaaattagtggttacttaacctggcttcagcagaaaccaga |
| | Optimized | | tggaactattaaacgcctgatctacgccgcatccactttagattctggtgtcccaaaaagg |
| | | | ttcagtggcagtaggtctgggtcagattattctctcaccatcagcagccttgagtctgaag |
| | | | attttgcagactataactgtctacaatatgctaattatcctcggacgttcggtggaggcacc |
| | | | aagctggaaatcaaac |
| 73 | Mouse 5E3 | VH | caggtccagctgcagcagtctggacctgagctggtgaagcctggggcttcagtgaggata |
| | Codon | | tcctgcaaggcttctggctacatattcacaagctactatatacagtgggtgatacacaggc |
| | Optimized | | ctggacagggacttgagtggattggatggatttatcctggaaatgttaatactaagtacaa |
| | | | tgagaagttcaagggcaaggccacactgactgcagacaaatcctccagcacagcctaca |
| | | | tgcagctcagcagattgacctctgaggactctgcggtctatttctgtgcaaggatggattac |
| | | | gaggctcactactggggccaaggcaccactctcacagtctcctcag |
| 74 | 5E3 CDR | VL | Gacattcagatgacccagagccctagttcactgagtgcctcagtcggggatcgagtgact |
| | Codon | | atcacctgtcgtgctagtcaggaaatttcaggttacctgacctggtatcagcagaagccag |
| | Optimized | | agaaagcccccaagagcctgatctacgctgcatccaccctggacagcggagtgccatctc |
| | | | gattctccggaagcgggtctggtacagactttacactgactatttccagcctgcagccaga |
| | | | ggatttcgcaacttactattgcctgcagtacgccaactatcccagaacctttggcggaggg |
| | | | acaaaagtggaaatcaagc |
| 75 | 5E3 CDR | VH | caggtgcagctggtccagtcaggcgcagaagtgaaaaaacccggagcatcagtcaaagt |
| | Codon | | ctcttgtaaggctagcggatatacattcacctcttactatatccagtgggtgagacaggctc |
| | Optimized | | caggacagcgcctggaatggatgggctggatctaccccggaaacgtcaatacaaagtat |
| | | | aacgagaagttcaaaggaagggtgactatcacccgggacacatcagcatccactgccta |
| | | | catggagctgtccagcctgagatccgaagacactgccgtgtactattgcgctcgcatggat |
| | | | tacgaagcccactattggggtcagggcactctggtcaccgtgtctagtg |
| 76 | 5E3 hu Codon | VL | gacattcagatgactcagtctcccagctctctgtcagcctccgtcggcgatagagtgacaa |
| | Optimized | | tcacttgtcgcgcttcccaggaaattagcggatacctgacttggctgcagcagaaacccg |
| | | | agggggccatcaagcgactgatctacgctgcatctaccctggacagtggagtgcctaaga |
| | | | ggttcagcggttctcggagtggctcagactactctctgactatcagttcactgcagcccga |
| | | | ggatttcgcaacctataactgcctgcagtacgccaattatcctagaacatttggcggaggg |
| | | | actaaactggaaatcaagc |
| 77 | 5E3 hu Codon | VH | caggtccagctgcagcagagcggtcccgaggtcaaaaaacccggcgcatccgtgaaaat |
| | Optimized | | cagttgtaaagcatccggttatatcttcacctcctactatatccagtgggtcatccacgcac |
| | | | ctggtcagggactggaatggatcggatggatctaccctgggaacgtgaatacaaagtata |
| | | | acgagaagttcaaaggcaaggctacactgactgcagacaagtccagctctactgcatac |
| | | | atggagctgagttcactgactagcgaagacaccgccgtgtatttctgcgctagaatggatt |
| | | | acgaagcccactattggggacaggggaccacagtcaccgtgtcctccg |
| 78 | 5E3 rehu | VL | gacattcagatgacacagagcccaagctctctgtcagcctccctgggcgacagagtgact |
| | Codon | | ctgacctgtcgcgcttctcaggaaatcagtggctacctgacatggctgcagcagaaaccc |
| | Optimized | | gagaaggccatcaaaagactgatctacgctgcatcaactctggactccggcgtgcctaag |
| | | | aggttcagcggttctcggagtggctcagattacacactgactattagttcactgcagcccg |
| | | | aggacttcgcagattataactgcctgcagtacgccaattatcctcgaacatttggcggagg |
| | | | gactaagctggaaatcaaac |
| 79 | 5E3 rehu | VH | caggtccagctgcagcagagcggtcccgaggtcgtgaaacccggagcatctgtgaaaat |
| | Codon | | cagttgtaaggccagcggatacatctttacctcttactatatccagtgggtcatccacgcac |
| | Optimized | | ctggtcagggactggaatggatcggatggatctaccctgggaacgtgaataccaagtata |
| | | | acgagaagttcaaaggcaaggctactctgaccgcagacaagtccagctctacagcatac |
| | | | atggagctgagttcactgaggtccgaagacagcgccgtgtatttctgcgctcggatggatt |
| | | | acgaagcccactattggggacaggggactctggtcaccgtgtcctccg |
| 80 | 5E3 chimeric | VL | gatattcagatgacccagagcccatcatccctgtctgccagtctgggcgagagagtgtctc |
| | Codon | | tgacctgtcgcgcttcccaggaaatcagcggatacctgacctggctgcagcagaaacccg |
| | Optimized | | acgggacaatcaagagactgatctacgctgcatctactctggatagtggagtgcctaaga |
| | | | ggttctcaggttcccggagcggctctgactacagtctgaccattagctctctggagtccga |
| | | | agacttcgcagattataactgcctgcagtacgccaattatccaagaacctttggcggagg |
| | | | gacaaaactggaaatcaagc |
| 81 | 5E3KCDR3-L89V | LCDR3 | GTG CAG TAC GCC AAT TAT CCT AGA ACA |
| 82 | 5E3KCDR3-L89V | LCDR3 | VQYANYPRT |
| 83 | 5E3KCDR3-L89I | LCDR3 | ATC CAG TAC GCC AAT TAT CCT AGA ACA |
| 84 | 5E3KCDR3-L89I | LCDR3 | IQYANYPRT |
| 85 | 5E3KCDR3-Q9ON | LCDR3 | CTG AAC TAC GCC AAT TAT CCT AGA ACA |
| 86 | 5E3KCDR3-Q9ON | LCDR3 | LNYANYPRT |
| 87 | 5E3KCDR3-Q90E | LCDR3 | CTG GAG TAC GCC AAT TAT CCT AGA ACA |
| 88 | 5E3KCDR3-Q90E | LCDR3 | LEYANYPRT |
| 89 | 5E3KCDR3-Y91F | LCDR3 | CTG CAG TTC GCC AAT TAT CCT AGA ACA |
| 90 | 5E3KCDR3-Y91F | LCDR3 | LQFANYPRT |
| 91 | 5E3KCDR3-N93Q | LCDR3 | CTG CAG TAC GCC CAG TAT CCT AGA ACA |
| 92 | 5E3KCDR3-N93Q | LCDR3 | LQYAQYPRT |
| 93 | 5E3KCDR3-N93D | LCDR3 | CTG CAG TAC GCC GAC TAT CCT AGA ACA |
| 94 | 5E3KCDR3-N93D | LCDR3 | LQYADYPRT |
| 95 | 5E3KCDR3-Y94F | LCDR3 | CTG CAG TAC GCC AAT TTC CCT AGA ACA |
| 96 | 5E3KCDR3-Y94F | LCDR3 | LQYANFPRT |
| 97 | 5E3HCDR3- | HCDR3 | gct aga AAG GAT TAC GAA GCC CAC TAT |
| | M206K | | |
| 98 | 5E3HCDR3- | HCDR3 | ARKDYEAHY |
| | M206K | | |
| 99 | 5E3HCDR3- | HCDR3 | gct aga ATC GAT TAC GAA GCC CAC TAT |
| | M206I | | |
| 100 | 5E3HCDR3- | HCDR3 | ARIDYEAHY |
| | M206I | | |
| 101 | 5E3HCDR3- | HCDR3 | gct aga ATG GAG TAC GAA GCC CAC TAT |
| | D207E | | |
| 102 | 5E3HCDR3- | HCDR3 | ARMEYEAHY |
| | D207E | | |
| 103 | 5E3HCDR3- | HCDR3 | gct aga ATG TCC TAC GAA GCC CAC TAT |
| | D207S | | |
| 104 | 5E3HCDR3- | HCDR3 | ARMSYEAHY |
| | D207S | | |
| 105 | 5E3HCDR3- | HCDR3 | gct aga ATG AAC TAC GAA GCC CAC TAT |
| | D207N | | |
| 106 | 5E3HCDR3- | HCDR3 | ARMNYEAHY |
| | D207N | | |
| 107 | 5E3HCDR3- | HCDR3 | gct aga ATG GAT TTC GAA GCC CAC TAT |
| | Y208F | | |
| 108 | 5E3HCDR3- | HCDR3 | ARMDFEAHY |
| | Y208F | | |
| 109 | 5E3HCDR3- | HCDR3 | gct aga ATG GAT TAC CAG GCC CAC TAT |
| | E209Q | | |
| 110 | 5E3HCDR3- | HCDR3 | ARMDYQAHY |
| | E209Q | | |
| 111 | 5E3HCDR3- | HCDR3 | gct aga ATG GAT TAC GAC GCC CAC TAT |
| | E209D | | |
| 112 | 5E3HCDR3- | HCDR3 | ARMDYDAHY |
| | E209D | | |
| 113 | 5E3HCDR3- | HCDR3 | gct aga ATG GAT TAC GAA GGC CAC TAT |
| | A210G | | |
| 114 | 5E3HCDR3- | HCDR3 | ARMDYEGHY |
| | A210G | | |
| 115 | 5E3HCDR3- | HCDR3 | gct aga ATG GAT TAC GAA GTC CAC TAT |
| | A210V | | |
| 116 | 5E3HCDR3- | HCDR3 | ARMDYEVHY |
| | A210V | | |
| 117 | 5E3HCDR3- | HCDR3 | gct aga ATG GAT TAC GAA GCC TTC TAT |
| | H211F | | |
| 118 | 5E3HCDR3- | HCDR3 | ARMDYEAFY |
| | H211F | | |
| 119 | 5E3HCDR3- | HCDR3 | gct aga ATG GAT TAC GAA GCC AAC TAT |
| | H211N | | |
| 120 | 5E3HCDR3- | HCDR3 | ARMDYEANY |
| | H211N | | |
| 121 | 5E3HCDR3- | HCDR3 | gct aga ATG GAT TAC GAA GCC CAC TTT |
| | Y212F | | |
| 122 | 5E3HCDR3- | HCDR3 | ARMDYEAHF |
| | Y212F | | |
| 123 | hu5E3 IHR | VH | caggtccagctgcagcagagcggtcccgaggtcaaaaaacccggcgcatccgtgaaaat |
| | Mutation | | cagttgtaaagcatccggttatatcttcacctcctactatatccagtgggtcatccacaggc |
| | | | ctggtcagggactggaatggatcggatggatctaccctgggaacgtgaatacaaagtata |
| | | | acgagaagttcaaaggcaaggctacactgactgcagacaagtccagctctactgcatac |
| | | | atggagctgagttcactgactagcgaagacaccgccgtgtatttctgcgctagaatggatt |
| | | | acgaagcccactattggggacaggggaccacagtcaccgtgtcctccg |
| 124 | huΔ5E3 IHR | VH | QVQLQQSGPEVKKPGASVKISCKASGYIFTSYYIQWVIHRPGQGLEWIG |
| | Mutation | | WIYPGNVNTKYNEKFKGKATLTADKSSSTAYMELSSLTSEDTAVYFCAR |
| | | | MDYEAHYWGQGTTVTVSS |
| 125 | hu5E3 KHA | VH | caggtccagctgcagcagagcggtcccgaggtcaaaaaacccggcgcatccgtgaaaat |
| | Mutation | | cagttgtaaagcatccggttatatcttcacctcctactatatccagtgggtcaagcacgcac |
| | | | ctggtcagggactggaatggatcggatggatctaccctgggaacgtgaatacaaagtata |
| | | | acgagaagttcaaaggcaaggctacactgactgcagacaagtccagctctactgcatac |
| | | | atggagctgagttcactgactagcgaagacaccgccgtgtatttctgcgctagaatggatt |
| | | | acgaagcccactattggggacaggggaccacagtcaccgtgtcctccg |
| 126 | huΔ5E3 KHA | VH | QVQLQQSGPEVKKPGASVKISCKASGYIFTSYYIQWVKHAPGQGLEWI |
| | Mutation | | GWIYPGNVNTKYNEKFKGKATLTADKSSSTAYMELSSLTSEDTAVYFCA |
| | | | RMDYEAHYWGQGTTVTVSS |
| 127 | hu5E3 KQR | VH | caggtccagctgcagcagagcggtcccgaggtcaaaaaacccggcgcatccgtgaaaat |
| | Mutation | | cagttgtaaagcatccggttatatcttcacctcctactatatccagtgggtcaagcagagg |
| | | | cctggtcagggactggaatggatcggatggatctaccctgggaacgtgaatacaaagtat |
| | | | aacgagaagttcaaaggcaaggctacactgactgcagacaagtccagctctactgcata |
| | | | catggagctgagttcactgactagcgaagacaccgccgtgtatttctgcgctagaatggat |
| | | | tacgaagcccactattggggacaggggaccacagtcaccgtgtcctccg |
| 128 | huΔ5E3 KQR | VH | QVQLQQSGPEVKKPGASVKISCKASGYIFTSYYIQWVKQRPGQGLEWI |
| | Mutation | | GWIYPGNVNTKYNEKFKGKATLTADKSSSTAYMELSSLTSEDTAVYFCA |
| | | | RMDYEAHYWGQGTTVTVSS |
| 129 | hu5E3 IQR | VH | caggtccagctgcagcagagcggtcccgaggtcaaaaaacccggcgcatccgtgaaaat |
| | Mutation | | cagttgtaaagcatccggttatatcttcacctcctactatatccagtgggtcatccagaggc |
| | | | ctggtcagggactggaatggatcggatggatctaccctgggaacgtgaatacaaagtata |
| | | | acgagaagttcaaaggcaaggctacactgactgcagacaagtccagctctactgcatac |
| | | | atggagctgagttcactgactagcgaagacaccgccgtgtatttctgcgctagaatggatt |
| | | | acgaagcccactattggggacaggggaccacagtcaccgtgtcctccg |
| 130 | huΔ5E3 IQR | VH | QVQLQQSGPEVKKPGASVKISCKASGYIFTSYYIQWVIQRPGQGLEWI |
| | Mutation | | GWIYPGNVNTKYNEKFKGKATLTADKSSSTAYMELSSLTSEDTAVYFCA |
| | | | RMDYEAHYWGQGTTVTVSS |
| 131 | hu5E3 KQA | VH | caggtccagctgcagcagagcggtcccgaggtcaaaaaacccggcgcatccgtgaaaat |
| | Mutation | | cagttgtaaagcatccggttatatcttcacctcctactatatccagtgggtcaagcaggca |
| | | | cctggtcagggactggaatggatcggatggatctaccctgggaacgtgaatacaaagtat |
| | | | aacgagaagttcaaaggcaaggctacactgactgcagacaagtccagctctactgcata |
| | | | catggagctgagttcactgactagcgaagacaccgccgtgtatttctgcgctagaatggat |
| | | | tacgaagcccactattggggacaggggaccacagtcaccgtgtcctccg |
| 132 | huA5E3 KQA | VH | QVQLQQSGPEVKKPGASVKISCKASGYIFTSYYIQWVKQAPGQGLEWI |
| | Mutation | | GWIYPGNVNTKYNEKFKGKATLTADKSSSTAYMELSSLTSEDTAVYFCA |
| | | | RMDYEAHYWGQGTTVTVSS |
| 133 | Murine 5E3 IHR | VH | CAGGTCCAGCTGCAGCAGTCTGGACCTGAGCTGGTGAAGCCTGGG |
| | Mutation | | GCTTCAGTGAGGATATCCTGCAAGGCTTCTGGCTACATATTCACAAG |
| | | | CTACTATAtacagtgggtgATCCACCGCcctggacagggacTTGAGTGGATT |
| | | | GGATGGATTTATCCTGGAAATGTTAATACTAAGTACAATGAGAAGT |
| | | | TCAAGGGCAAGGCCACACTGACTGCAGACAAATCCTCCAGCACAGC |
| | | | CTACATGCAGCTCAGCAGATTGACCTCTGAGGACTCTGCGGTCTATT |
| | | | TCTGTGCAAGGATGGATTACGAGGCTCACTACTGGGGCCAAGGCAC |
| | | | CACTCTCACAGTCTCCTCA |
| 134 | Murine 5E3 IHR | VH | QVQLQQSGPELVKPGASVRISCKASGYIFTSYYIQWVIHRPGQGLEWIG |
| | Mutation | | WIYPGNVNTKYNEKFKGKATLTADKSSSTAYMQLSRLTSEDSAVYFCAR |
| | | | MDYEAHYWGQGTTLTVSS |
| 135 | Murine 5E3 | VH | CAGGTCCAGCTGCAGCAGTCTGGACCTGAGCTGGTGAAGCCTGGG |
| | KQA Mutation | | GCTTCAGTGAGGATATCCTGCAAGGCTTCTGGCTACATATTCACAAG |
| | | | CTACTATAtacagtgggtgAAGCAGGCCcctggacagggacTTGAGTGGATT |
| | | | GGATGGATTTATCCTGGAAATGTTAATACTAAGTACAATGAGAAGT |
| | | | TCAAGGGCAAGGCCACACTGACTGCAGACAAATCCTCCAGCACAGC |
| | | | CTACATGCAGCTCAGCAGATTGACCTCTGAGGACTCTGCGGTCTATT |
| | | | TCTGTGCAAGGATGGATTACGAGGCTCACTACTGGGGCCAAGGCAC |
| | | | CACTCTCACAGTCTCCTCA |
| 136 | Murine 5E3 | VH | QVQLQQSGPELVKPGASVRISCKASGYIFTSYYIQWVKQAPGQGLEWI |
| | KQA Mutation | | GWIYPGNVNTKYNEKFKGKATLTADKSSSTAYMQLSRLTSEDSAVYFC |
| | | | ARMDYEAHYWGQGTTLTVSS |
| 137 | Murine 5E3 | VH | CAGGTCCAGCTGCAGCAGTCTGGACCTGAGCTGGTGAAGCCTGGG |
| | KHA Mutation | | GCTTCAGTGAGGATATCCTGCAAGGCTTCTGGCTACATATTCACAAG |
| | | | CTACTATAtacagtgggtgAAGCACGCCcctggacagggacTTGAGTGGATT |
| | | | GGATGGATTTATCCTGGAAATGTTAATACTAAGTACAATGAGAAGT |
| | | | TCAAGGGCAAGGCCACACTGACTGCAGACAAATCCTCCAGCACAGC |
| | | | CTACATGCAGCTCAGCAGATTGACCTCTGAGGACTCTGCGGTCTATT |
| | | | TCTGTGCAAGGATGGATTACGAGGCTCACTACTGGGGCCAAGGCAC |
| | | | CACTCTCACAGTCTCCTCA |
| 138 | Murine 5E3 | VH | QVQLQQSGPELVKPGASVRISCKASGYIFTSYYIQWVKHAPGQGLEWI |
| | KHA Mutation | | GWIYPGNVNTKYNEKFKGKATLTADKSSSTAYMQLSRLTSEDSAVYFC |
| | | | ARMDYEAHYWGQGTTLTVSS |
| 139 | Murine 5E3 | VH | caggtccagctgcagcagtctggacctgagctggtgaagcctggggcttcagtgaggata |
| | KQR Mutation | | tcctgcaaggcttctggctacatattcacaagctactatatacagtgggtgaagcagaggc |
| | | | ctggacagggacttgagtggattggatggatttatcctggaaatgttaatactaagtacaa |
| | | | tgagaagttcaagggcaaggccacactgactgcagacaaatcctccagcacagcctaca |
| | | | tgcagctcagcagattgacctctgaggactctgcggtctatttctgtgcaaggatggattac |
| | | | gaggctcactactggggccaaggcaccactctcacagtctcctcag |
| 140 | Murine 5E3 | VH | QVQLQQSGPELVKPGASVRISCKASGYIFTSYYIQWVKQRPGQGLEWI |
| | KQR Mutation | | GWIYPGNVNTKYNEKFKGKATLTADKSSSTAYMQLSRLTSEDSAVYFC |
| | | | ARMDYEAHYWGQGTTLTVSS |
| 141 | Murine 5E3 IQR | VH | CAGGTCCAGCTGCAGCAGTCTGGACCTGAGCTGGTGAAGCCTGGG |
| | Mutation | | GCTTCAGTGAGGATATCCTGCAAGGCTTCTGGCTACATATTCACAAG |
| | | | CTACTATAtacagtgggtgATCCAGCGCcctggacagggacTTGAGTGGATT |
| | | | GGATGGATTTATCCTGGAAATGTTAATACTAAGTACAATGAGAAGT |
| | | | TCAAGGGCAAGGCCACACTGACTGCAGACAAATCCTCCAGCACAGC |
| | | | CTACATGCAGCTCAGCAGATTGACCTCTGAGGACTCTGCGGTCTATT |
| | | | TCTGTGCAAGGATGGATTACGAGGCTCACTACTGGGGCCAAGGCAC |
| | | | CACTCTCACAGTCTCCTCA |
| 142 | Murine 5E3 IQR | VH | QVQLQQSGPELVKPGASVRISCKASGYIFTSYYIQWVIQRPGQGLEWIG |
| | Mutation | | WIYPGNVNTKYNEKFKGKATLTADKSSSTAYMQLSRLTSEDSAVYFCAR |
| | | | MDYEAHYWGQGTTLTVSS |
| 143 | 5E3 CDR IHR | VH | CAGGTGCAGCTGGTCCAGTCAGGCGCAGAAGTGAAAAAACCCGGA |
| | Mutation | | GCATCAGTCAAAGTCTCTTGTAAGGCTAGCGGATATACATTCACCTC |
| | | | TTACTATATCCAGTGGGTGATCCACCGCCCAGGACAGCGCCTGGAA |
| | | | TGGATGGGCTGGATCTACCCCGGAAACGTCAATACAAAGTATAACG |
| | | | AGAAGTTCAAAGGAAGGGTGACTATCACCCGGGACACATCAGCATC |
| | | | CACTGCCTACATGGAGCTGTCCAGCCTGAGATCCGAAGACACTGCC |
| | | | GTGTACTATTGCGCTCGCATGGATTACGAAGCCCACTATTGGGGTC |
| | | | AGGGCACTCTGGTCACCGTGTCTAGT |
| 144 | 5E3 CDR IHR | VH | QVQLVQSGAEVKKPGASVINSCKASGYTFTSYYIQWVIHRPGQRLEW |
| | Mutation | | MGWIYPGNVNTKYNEKFKGRVTITRDTSASTAYMELSSLRSEDTAVYY |
| | | | CARMDYEAHYWGQGTLVTVSS |
| 145 | 5E3 CDR KQA | VH | CAGGTGCAGCTGGTCCAGTCAGGCGCAGAAGTGAAAAAACCCGGA |
| | Mutation | | GCATCAGTCAAAGTCTCTTGTAAGGCTAGCGGATATACATTCACCTC |
| | | | TTACTATATCcagtgggtgAAGCAGGCCccaggacagcgcctgGAATGGAT |
| | | | GGGCTGGATCTACCCCGGAAACGTCAATACAAAGTATAACGAGAAG |
| | | | TTCAAAGGAAGGGTGACTATCACCCGGGACACATCAGCATCCACTG |
| | | | CCTACATGGAGCTGTCCAGCCTGAGATCCGAAGACACTGCCGTGTA |
| | | | CTATTGCGCTCGCATGGATTACGAAGCCCACTATTGGGGTCAGGGC |
| | | | ACTCTGGTCACCGTGTCTAGT |
| 146 | 5E3 CDR KQA | VH | QVQLVQSGAEVKKPGASVINSCKASGYTFTSYYIQWVKQAPGQRLEW |
| | Mutation | | MGWIYPGNVNTKYNEKFKGRVTITRDTSASTAYMELSSLRSEDTAVYY |
| | | | CARMDYEAHYWGQGTLVTVSS |
| 147 | 5E3 CDR KHA | VH | CAGGTGCAGCTGGTCCAGTCAGGCGCAGAAGTGAAAAAACCCGGA |
| | Mutation | | GCATCAGTCAAAGTCTCTTGTAAGGCTAGCGGATATACATTCACCTC |
| | | | TTACTATATCcagtgggtgAAGCACGCCccaggacagcgcctgGAATGGAT |
| | | | GGGCTGGATCTACCCCGGAAACGTCAATACAAAGTATAACGAGAAG |
| | | | TTCAAAGGAAGGGTGACTATCACCCGGGACACATCAGCATCCACTG |
| | | | CCTACATGGAGCTGTCCAGCCTGAGATCCGAAGACACTGCCGTGTA |
| | | | CTATTGCGCTCGCATGGATTACGAAGCCCACTATTGGGGTCAGGGC |
| | | | ACTCTGGTCACCGTGTCTAGT |
| 148 | 5E3 CDR KHA | VH | QVQLVQSGAEVKKPGASVINSCKASGYTFTSYYIQWVKHAPGQRLEW |
| | Mutation | | MGWIYPGNVNTKYNEKFKGRVTITRDTSASTAYMELSSLRSEDTAVYY |
| | | | CARMDYEAHYWGQGTLVTVSS |
| 149 | 5E3 CDR KQR | VH | CAGGTGCAGCTGGTCCAGTCAGGCGCAGAAGTGAAAAAACCCGGA |
| | Mutation | | GCATCAGTCAAAGTCTCTTGTAAGGCTAGCGGATATACATTCACCTC |
| | | | TTACTATATCcagtgggtgAAGCAGCGCccaggacagcgcctgGAATGGAT |
| | | | GGGCTGGATCTACCCCGGAAACGTCAATACAAAGTATAACGAGAAG |
| | | | TTCAAAGGAAGGGTGACTATCACCCGGGACACATCAGCATCCACTG |
| | | | CCTACATGGAGCTGTCCAGCCTGAGATCCGAAGACACTGCCGTGTA |
| | | | CTATTGCGCTCGCATGGATTACGAAGCCCACTATTGGGGTCAGGGC |
| | | | ACTCTGGTCACCGTGTCTAGT |
| 150 | 5E3 CDR KQR | VH | QVQLVQSGAEVKKPGASVINSCKASGYTFTSYYIQWVKQRPGQRLEW |
| | Mutation | | MGWIYPGNVNTKYNEKFKGRVTITRDTSASTAYMELSSLRSEDTAVYY |
| | | | CARMDYEAHYWGQGTLVTVSS |
| 151 | 5E3 CDR IQR | VH | CAGGTGCAGCTGGTCCAGTCAGGCGCAGAAGTGAAAAAACCCGGA |
| | Mutation | | GCATCAGTCAAAGTCTCTTGTAAGGCTAGCGGATATACATTCACCTC |
| | | | TTACTATATCcagtgggtgATCCAGCGCccaggacagcgcctgGAATGGATG |
| | | | GGCTGGATCTACCCCGGAAACGTCAATACAAAGTATAACGAGAAGT |
| | | | TCAAAGGAAGGGTGACTATCACCCGGGACACATCAGCATCCACTGC |
| | | | CTACATGGAGCTGTCCAGCCTGAGATCCGAAGACACTGCCGTGTAC |
| | | | TATTGCGCTCGCATGGATTACGAAGCCCACTATTGGGGTCAGGGCA |
| | | | CTCTGGTCACCGTGTCTAGT |
| 152 | 5E3 CDR IQR | VH | QVQLVQSGAEVKKPGASVINSCKASGYTFTSYYIQWVIQRPGQRLEW |
| | Mutation | | MGWIYPGNVNTKYNEKFKGRVTITRDTSASTAYMELSSLRSEDTAVYY |
| | | | CARMDYEAHYWGQGTLVTVSS |
| 153 | 5E3 rehu IHR | VH | CAGGTCCAGCTGCAGCAGAGCGGTCCCGAGGTCGTGAAACCCGGA |
| | Mutation | | GCATCTGTGAAAATCAGTTGTAAGGCCAGCGGATACATCTTTACCTC |
| | | | TTACTATAtccagtgggtcATCCACCGCcctggtcagggactggaatggatcGGA |
| | | | TGGATCTACCCTGGGAACGTGAATACCAAGTATAACGAGAAGTTCA |
| | | | AAGGCAAGGCTACTCTGACCGCAGACAAGTCCAGCTCTACAGCATA |
| | | | CATGGAGCTGAGTTCACTGAGGTCCGAAGACAGCGCCGTGTATTTC |
| | | | TGCGCTCGGATGGATTACGAAGCCCACTATTGGGGACAGGGGACTC |
| | | | TGGTCACCGTGTCCTCC |
| 154 | 5E3 rehu IHR | VH | QVQLQQSGPEVVKPGASVKISCKASGYIFTSYYIQWVIHRPGQGLEWI |
| | Mutation | | GWIYPGNVNTKYNEKFKGKATLTADKSSSTAYMELSSLRSEDSAVYFCA |
| | | | RMDYEAHYWGQGTLVTVSS |
| 155 | 5E3 rehu KQA | VH | CAGGTCCAGCTGCAGCAGAGCGGTCCCGAGGTCGTGAAACCCGGA |
| | Mutation | | GCATCTGTGAAAATCAGTTGTAAGGCCAGCGGATACATCTTTACCTC |
| | | | TTACTATAtccagtgggtcAAGCAGGCCcctggtcagggactggaatggatcGG |
| | | | ATGGATCTACCCTGGGAACGTGAATACCAAGTATAACGAGAAGTTC |
| | | | AAAGGCAAGGCTACTCTGACCGCAGACAAGTCCAGCTCTACAGCAT |
| | | | ACATGGAGCTGAGTTCACTGAGGTCCGAAGACAGCGCCGTGTATTT |
| | | | CTGCGCTCGGATGGATTACGAAGCCCACTATTGGGGACAGGGGACT |
| | | | CTGGTCACCGTGTCCTCC |
| 156 | 5E3 rehu KQA | VH | QVQLQQSGPEVVKPGASVKISCKASGYIFTSYYIQWVKQAPGQGLEWI |
| | Mutation | | GWIYPGNVNTKYNEKFKGKATLTADKSSSTAYMELSSLRSEDSAVYFCA |
| | | | RMDYEAHYWGQGTLVTVSS |
| 157 | 5E3 rehu KHA | VH | CAGGTCCAGCTGCAGCAGAGCGGTCCCGAGGTCGTGAAACCCGGA |
| | Mutation | | GCATCTGTGAAAATCAGTTGTAAGGCCAGCGGATACATCTTTACCTC |
| | | | TTACTATAtccagtgggtcAAGCACGCCcctggtcagggactggaatggatcGG |
| | | | ATGGATCTACCCTGGGAACGTGAATACCAAGTATAACGAGAAGTTC |
| | | | AAAGGCAAGGCTACTCTGACCGCAGACAAGTCCAGCTCTACAGCAT |
| | | | ACATGGAGCTGAGTTCACTGAGGTCCGAAGACAGCGCCGTGTATTT |
| | | | CTGCGCTCGGATGGATTACGAAGCCCACTATTGGGGACAGGGGACT |
| | | | CTGGTCACCGTGTCCTCC |
| 158 | 5E3 rehu KHA | VH | QVQLQQSGPEVVKPGASVKISCKASGYIFTSYYIQWVKHAPGQGLEWI |
| | Mutation | | GWIYPGNVNTKYNEKFKGKATLTADKSSSTAYMELSSLRSEDSAVYFCA |
| | | | RMDYEAHYWGQGTLVTVSS |
| 159 | 5E3 rehu KQR | VH | CAGGTCCAGCTGCAGCAGAGCGGTCCCGAGGTCGTGAAACCCGGA |
| | Mutation | | GCATCTGTGAAAATCAGTTGTAAGGCCAGCGGATACATCTTTACCTC |
| | | | TTACTATAtccagtgggtcAAGCAGCGCcctggtcagggactggaatggatcGG |
| | | | ATGGATCTACCCTGGGAACGTGAATACCAAGTATAACGAGAAGTTC |
| | | | AAAGGCAAGGCTACTCTGACCGCAGACAAGTCCAGCTCTACAGCAT |
| | | | ACATGGAGCTGAGTTCACTGAGGTCCGAAGACAGCGCCGTGTATTT |
| | | | CTGCGCTCGGATGGATTACGAAGCCCACTATTGGGGACAGGGGACT |
| | | | CTGGTCACCGTGTCCTCC |
| 160 | 5E3 rehu KQR | VH | QVQLQQSGPEVVKPGASVKISCKASGYIFTSYYIQWVKQRPGQGLEWI |
| | Mutation | | GWIYPGNVNTKYNEKFKGKATLTADKSSSTAYMELSSLRSEDSAVYFCA |
| | | | RMDYEAHYWGQGTLVTVSS |
| 161 | 5E3 rehu IQR | VH | CAGGTCCAGCTGCAGCAGAGCGGTCCCGAGGTCGTGAAACCCGGA |
| | Mutation | | GCATCTGTGAAAATCAGTTGTAAGGCCAGCGGATACATCTTTACCTC |
| | | | TTACTATAtccagtgggtcATCCAGCGCcctggtcagggactggaatggatcGGA |
| | | | TGGATCTACCCTGGGAACGTGAATACCAAGTATAACGAGAAGTTCA |
| | | | AAGGCAAGGCTACTCTGACCGCAGACAAGTCCAGCTCTACAGCATA |
| | | | CATGGAGCTGAGTTCACTGAGGTCCGAAGACAGCGCCGTGTATTTC |
| | | | TGCGCTCGGATGGATTACGAAGCCCACTATTGGGGACAGGGGACTC |
| | | | TGGTCACCGTGTCCTCC |
| 162 | 5E3 rehu IQR | VH | QVQLQQSGPEVVKPGASVKISCKASGYIFTSYYIQWVIQRPGQGLEWI |
| | Mutation | | GWIYPGNVNTKYNEKFKGKATLTADKSSSTAYMELSSLRSEDSAVYFCA |
| | | | RMDYEAHYWGQGTLVTVSS |
| 163 | 5E3-R48D-AFF | LCDR1 | GACGCTTCCCAGGAAATTAGCGGATACCTGACT |
| 164 | 5E3-R48D-AFF | LCDR1 | DASQEISGYLT |
| 165 | 5E3-S50D-AFF | LCDR1 | CGCGCGACCCAGGAAATTAGCGGATACCTGACT |
| 166 | 5E3-S50D-AFF | LCDR1 | RATQEISGYLT |
| 167 | 5E3-Q51D-AFF | LCDR1 | GACGAAATTAGCGGATAC |
| 168 | 5E3-Q51D-AFF | LCDR1 | DEISGY |
| 169 | 5E3-E52D-AFF | LCDR1 | CAGGACATTAGCGGATAC |
| 170 | 5E3-E52D-AFF | LCDR1 | QDISGY |
| 171 | 5E3-S54D-AFF | LCDR1 | CAGGAAATTGACGGATAC |
| 172 | 5E3-S54D-AFF | LCDR1 | QEIDGY |
| 173 | 5E3-A74D-AFF | LCDR2 | GACGCATCTACCCTGGACAGTGGA |
| 174 | 5E3-A74D-AFF | LCDR2 | DASTLDSG |
| 175 | 5E3-S76D-AFF | LCDR2 | GCTGCAGACACCCTGGACAGTGGA |
| 176 | 5E3-S76D-AFF | LCDR2 | AADTLDSG |
| 177 | 5E3-T77D-AFF | LCDR2 | GCTGCATCTGACCTGGACAGTGGA |
| 178 | 5E3-T77D-AFF | LCDR2 | AASDLDSG |
| 179 | 5E3-L78D-AFF | LCDR2 | GCTGCATCTACCGACGACAGTGGA |
| 180 | 5E3-L78D-AFF | LCDR2 | AASTDDSG |
| 181 | 5E3-S80D-AFF | LCDR2 | GCTGCATCTACCCTGGACGACGGA |
| 182 | 5E3-S80D-AFF | LCDR2 | AASTLDDG |
| 183 | 5E3-G81D-AFF | LCDR2 | GCTGCATCTACCCTGGACAGTGAC |
| 184 | 5E3-G81D-AFF | LCDR2 | AASTLDSD |
| 185 | 5E3-A116D-AFF | LCDR3 | CTGCAGTACGACAATTATCCTAGAACA |
| 186 | 5E3-A116D-AFF | LCDR3 | QYDNYPRT |
| 187 | 5E3-N117D-AFF | LCDR3 | CTGCAGTACGCCGACTATCCTAGAACA |
| 188 | 5E3-N117D-AFF | LCDR3 | QYADYPRT |
| 189 | 5E3-T183D-AFF | HCDR1 | ggatatatattcGACTCCTACTAT |
| 190 | 5E3-T183D-AFF | HCDR1 | GYIFDSYY |
| 191 | 5E3-S184D-AFF | HCDR1 | ggatatatattcACCGACTACTAT |
| 192 | 5E3-S184D-AFF | HCDR1 | GYIFDTYY |
| 193 | 5E3-G207D-AFF | HCDR2 | ATCTACCCTGACAACGTGAATACA |
| 194 | 5E3-G207D-AFF | HCDR2 | IYPDNVNT |
| 195 | 5E3-N208D-AFF | HCDR2 | ATCTACCCTGGGGACGTGAATACA |
| 196 | 5E3-N208D-AFF | HCDR2 | IYPGDVNT |
| 197 | 5E3-V209D-AFF | HCDR2 | ATCTACCCTGGGAACGACAATACA |
| 198 | 5E3-V209D-AFF | HCDR2 | IYPGNDNT |
| 199 | 5E3-N210D-AFF | HCDR2 | ATCTACCCTGGGAACGTGGACACA |
| 200 | 5E3-N210D-AFF | HCDR2 | IYPGNVDT |
| 201 | 5E3-T211D-AFF | HCDR2 | ATCTACCCTGGGAACGTGAATGAC |
| 202 | 5E3-T211D-AFF | HCDR2 | IYPGNVND |
| 203 | 5E3-K212D-AFF | HCDR2 | TGGATCTACCCTGGGAACGTGAATACAGACTATAACGAGAAGTTCA |
| | | | AA |
| 204 | 5E3-K212D-AFF | HCDR2 | WIYPGNVNTDYNEKFK |
| 205 | 5E3-E255D-AFF | HCDR3 | gct aga ATGGATTACGACGCCCACTAT |
| 206 | 5E3-E255D-AFF | HCDR3 | ARMDYDAHY |
| 207 | HuA5E3-IgG1 | IgG1 hc | MACPGFLWALVISTCLEFSMAQVQLQQSGPEVKKPGASVKISCKASGY |
| | (KHA) | | IFTSYYIQWVKHAPGQGLEWIGWIYPGNVNTKYNEKFKGKATLTADKS |
| | | | SSTAYMELSSLTSEDTAVYFCARMDYEAHYWGQGTTVTVSSASTKGPS |
| | | | VFPLAPSSKSTSGGTAALGCLVKDYFPEPVTVSWNSGALTSGVHTFPAV |
| | | | LQSSGLYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSHEDPE |
| | | | VKFNWYVDGVEVHNAKTKPREEQYNSTYRVVSVLTVLHQDWLNGKE |
| | | | YKCKVSNKALPAPIEKTISKAKGQPREPQVYTLPPSRDELTKNQVSLTCL |
| | | | VKGFYPSDIAVEWESNGQPENNYKTIPPVLDSDGSFFLYSKLTVDKSR |
| | | | WQQGNVFSCSVMHEALHNHYTQKSLSLSPGK |
| 208 | HuA5E3-IgG1 | IgG1 hc | ATGGCATGCCCTGGCTTCCTGTGGGCACTTGTGATCTCCACCTGTCT |
| | (KHA) | | TGAATTTTCCATGGCTCAGGTCCAGCTGCAGCAGAGCGGTCCCGAG |
| | | | GTCAAAAAACCCGGCGCATCCGTGAAAATCAGTTGTAAAGCATCCG |
| | | | GTTATATCTTCACCTCCTACTATATCCAGTGGGTCAAGCACGCACCT |
| | | | GGTCAGGGACTGGAATGGATCGGATGGATCTACCCTGGGAACGTG |
| | | | AATACAAAGTATAACGAGAAGTTCAAAGGCAAGGCTACACTGACTG |
| | | | CAGACAAGTCCAGCTCTACTGCATACATGGAGCTGAGTTCACTGACT |
| | | | AGCGAAGACACCGCCGTGTATTTCTGCGCTAGAATGGATTACGAAG |
| | | | CCCACTATTGGGGACAGGGGACCACAGTCACCGTGTCCTCCGCCAG |
| | | | CACAAAAGGTCCTTCCGTGTTCCCTCTGGCACCATCTAGTAAGTCTA |
| | | | CAAGTGGCGGAACTGCCGCTCTGGGCTGTCTGGTGAAGGATTACTT |
| | | | CCCTGAGCCAGTCACCGTGTCCTGGAACAGCGGTGCACTGACTTCT |
| | | | GGCGTCCATACCTTTCCAGCCGTGCTGCAGTCATCCGGACTGTACTC |
| | | | CCTGAGCTCTGTGGTCACTGTCCCCAGTTCATCCCTGGGGACCCAGA |
| | | | CATATATCTGCAACGTGAATCACAAACCTTCTAATACAAAGGTCGAC |
| | | | AAGAAAGTGGAACCAAAATCCTGTGATAAGACTCATACCTGCCCAC |
| | | | CTTGTCCAGCTCCTGAGCTGCTGGGAGGTCCAAGCGTGTTCCTGTTT |
| | | | CCACCCAAACCCAAGGACACCCTGATGATTAGCCGGACCCCTGAAG |
| | | | TCACATGCGTGGTCGTGGACGTGTCTCACGAGGATCCAGAAGTCAA |
| | | | GTTCAACTGGTACGTGGATGGCGTCGAGGTGCATAATGCCAAAACC |
| | | | AAGCCACGAGAGGAACAGTACAACAGTACATATCGTGTCGTGTCAG |
| | | | TCCTGACTGTGCTGCACCAGGACTGGCTGAACGGAAAGGAGTATAA |
| | | | ATGCAAGGTGTCCAACAAGGCCCTGCCAGCCCCCATCGAGAAGACC |
| | | | ATTAGCAAAGCTAAGGGGCAGCCCAGGGAACCTCAGGTGTACACA |
| | | | CTGCCTCCAAGTCGGGACGAGCTGACTAAAAACCAGGTCAGCCTGA |
| | | | CCTGTCTGGTGAAGGGTTTTTATCCAAGCGATATCGCAGTGGAGTG |
| | | | GGAATCTAATGGCCAGCCCGAGAACAATTACAAGACTACCCCCCCT |
| | | | GTGCTGGACTCTGATGGTAGTTTCTTTCTGTATTCTAAACTGACCGT |
| | | | GGATAAGAGTAGGTGGCAGCAGGGCAACGTCTTCTCATGCTCCGTG |
| | | | ATGCATGAAGCTCTGCACAATCATTACACCCAGAAAAGCCTGTCTCT |
| | | | GAGTCCTGGAAAGTGATAA |
| 209 | HuA5E3-IgG1 | IgG1 hc | MACPGFLWALVISTCLEFSMAQVQLQQSGPEVKKPGASVKISCKASGY |
| | (IHR) | | IFTSYYIQWVIHRPGQGLEWIGWIYPGNVNTKYNEKFKGKATLTADKSS |
| | | | STAYMELSSLTSEDTAVYFCARMDYEAHYWGQGTTVTVSSASTKGPSV |
| | | | FPLAPSSKSTSGGTAALGCLVKDYFPEPVTVSWNSGALTSGVHTFPAVL |
| | | | QSSGLYSLSSVVTVPSSSLGTQTYICNVNHKPSNTINDKINEPKSCDKTH |
| | | | TCPPCPAPELLGGPSVFLFPPKPKDTLMISRTPEVTCVVVDVSHEDPEVK |
| | | | FNWYVDGVEVHNAKTKPREEQYNSTYRVVSVLTVLHQDWLNGKEYK |
| | | | CINSNKALPAPIEKTISKAKGQPREPQVYTLPPSRDELTKNQVSLTCLVK |
| | | | GFYPSDIAVEWESNGQPENNYKTTPPVLDSDGSFFLYSKLTVDKSRWQ |
| | | | QGNVFSCSVMHEALHNHYTQKSLSLSPGK.. |
| 210 | HuA5E3-IgG1 | IgG1 hc | ATGGCATGCCCTGGCTTCCTGTGGGCACTTGTGATCTCCACCTGTCTTGAAT |
| | (IHR) | | TTTCCATGGCTCAGGTCCAGCTGCAGCAGAGCGGTCCCGAGGTCAAAAAAC |
| | | | CCGGCGCATCCGTGAAAATCAGTTGTAAAGCATCCGGTTATATCTTCACCTC |
| | | | CTACTATATCCAGTGGGTCATCCACAGGCCTGGTCAGGGACTGGAATGGAT |
| | | | CGGATGGATCTACCCTGGGAACGTGAATACAAAGTATAACGAGAAGTTCA |
| | | | AAGGCAAGGCTACACTGACTGCAGACAAGTCCAGCTCTACTGCATACATGG |
| | | | AGCTGAGTTCACTGACTAGCGAAGACACCGCCGTGTATTTCTGCGCTAGAA |
| | | | TGGATTACGAAGCCCACTATTGGGGACAGGGGACCACAGTCACCGTGTCCT |
| | | | CCGCCAGCACAAAAGGTCCTTCCGTGTTCCCTCTGGCACCATCTAGTAAGTC |
| | | | TACAAGTGGCGGAACTGCCGCTCTGGGCTGTCTGGTGAAGGATTACTTCCC |
| | | | TGAGCCAGTCACCGTGTCCTGGAACAGCGGTGCACTGACTTCTGGCGTCCA |
| | | | TACCTTTCCAGCCGTGCTGCAGTCATCCGGACTGTACTCCCTGAGCTCTGTG |
| | | | GTCACTGTCCCCAGTTCATCCCTGGGGACCCAGACATATATCTGCAACGTGA |
| | | | ATCACAAACCTTCTAATACAAAGGTCGACAAGAAAGTGGAACCAAAATCCT |
| | | | GTGATAAGACTCATACCTGCCCACCTTGTCCAGCTCCTGAGCTGCTGGGAG |
| | | | GTCCAAGCGTGTTCCTGTTTCCACCCAAACCCAAGGACACCCTGATGATTAG |
| | | | CCGGACCCCTGAAGTCACATGCGTGGTCGTGGACGTGTCTCACGAGGATCC |
| | | | AGAAGTCAAGTTCAACTGGTACGTGGATGGCGTCGAGGTGCATAATGCCA |
| | | | AAACCAAGCCACGAGAGGAACAGTACAACAGTACATATCGTGTCGTGTCA |
| | | | GTCCTGACTGTGCTGCACCAGGACTGGCTGAACGGAAAGGAGTATAAATG |
| | | | CAAGGTGTCCAACAAGGCCCTGCCAGCCCCCATCGAGAAGACCATTAGCAA |
| | | | AGCTAAGGGGCAGCCCAGGGAACCTCAGGTGTACACACTGCCTCCAAGTC |
| | | | GGGACGAGCTGACTAAAAACCAGGTCAGCCTGACCTGTCTGGTGAAGGGT |
| | | | TTTTATCCAAGCGATATCGCAGTGGAGTGGGAATCTAATGGCCAGCCCGAG |
| | | | AACAATTACAAGACTACCCCCCCTGTGCTGGACTCTGATGGTAGTTTCTTTC |
| | | | TGTATTCTAAACTGACCGTGGATAAGAGTAGGTGGCAGCAGGGCAACGTC |
| | | | TTCTCATGCTCCGTGATGCATGAAGCTCTGCACAATCATTACACCCAGAAAA |
| | | | GCCTGTCTCTGAGTCCTGGAAAGTGATAA |
| 211 | HuA5E3-IgG1 | IgG1 hc | MACPGFLWALVISTCLEFSMAQVQLQQSGPEVKKPGASVKISCKASGY |
| | (KQA) | | IFTSYYIQWVKQAPGQGLEWIGWIYPGNVNTKYNEKFKGKATLTADKS |
| | | | SSTAYMELSSLTSEDTAVYFCARMDYEAHYWGQGTTVTVSSASTKGPS |
| | | | VFPLAPSSKSTSGGTAALGCLVKDYFPEPVTVSWNSGALTSGVHTFPAV |
| | | | LQSSGLYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSHEDPE |
| | | | VKFNWYVDGVEVHNAKTKPREEQYNSTYRVVSVLTVLHQDWLNGKE |
| | | | YKCKVSNKALPAPIEKTISKAKGQPREPQVYTLPPSRDELTKNQVSLTCL |
| | | | VKGFYPSDIAVEWESNGQPENNYKTIPPVLDSDGSFFLYSKLTVDKSR |
| | | | WQQGNVFSCSVMHEALHNHYTQKSLSLSPGK |
| 212 | HuA5E3-IgG1 | IgG1 hc | ATGGCATGCCCTGGCTTCCTGTGGGCACTTGTGATCTCCACCTGTCTTGAAT |
| | (KQA) | | TTTCCATGGCTCAGGTCCAGCTGCAGCAGAGCGGTCCCGAGGTCAAAAAAC |
| | | | CCGGCGCATCCGTGAAAATCAGTTGTAAAGCATCCGGTTATATCTTCACCTC |
| | | | CTACTATATCCAGTGGGTCAAGCAGGCACCTGGTCAGGGACTGGAATGGA |
| | | | TCGGATGGATCTACCCTGGGAACGTGAATACAAAGTATAACGAGAAGTTCA |
| | | | AAGGCAAGGCTACACTGACTGCAGACAAGTCCAGCTCTACTGCATACATGG |
| | | | AGCTGAGTTCACTGACTAGCGAAGACACCGCCGTGTATTTCTGCGCTAGAA |
| | | | TGGATTACGAAGCCCACTATTGGGGACAGGGGACCACAGTCACCGTGTCCT |
| | | | CCGCCAGCACAAAAGGTCCTTCCGTGTTCCCTCTGGCACCATCTAGTAAGTC |
| | | | TACAAGTGGCGGAACTGCCGCTCTGGGCTGTCTGGTGAAGGATTACTTCCC |
| | | | TGAGCCAGTCACCGTGTCCTGGAACAGCGGTGCACTGACTTCTGGCGTCCA |
| | | | TACCTTTCCAGCCGTGCTGCAGTCATCCGGACTGTACTCCCTGAGCTCTGTG |
| | | | GTCACTGTCCCCAGTTCATCCCTGGGGACCCAGACATATATCTGCAACGTGA |
| | | | ATCACAAACCTTCTAATACAAAGGTCGACAAGAAAGTGGAACCAAAATCCT |
| | | | GTGATAAGACTCATACCTGCCCACCTTGTCCAGCTCCTGAGCTGCTGGGAG |
| | | | GTCCAAGCGTGTTCCTGTTTCCACCCAAACCCAAGGACACCCTGATGATTAG |
| | | | CCGGACCCCTGAAGTCACATGCGTGGTCGTGGACGTGTCTCACGAGGATCC |
| | | | AGAAGTCAAGTTCAACTGGTACGTGGATGGCGTCGAGGTGCATAATGCCA |
| | | | AAACCAAGCCACGAGAGGAACAGTACAACAGTACATATCGTGTCGTGTCA |
| | | | GTCCTGACTGTGCTGCACCAGGACTGGCTGAACGGAAAGGAGTATAAATG |
| | | | CAAGGTGTCCAACAAGGCCCTGCCAGCCCCCATCGAGAAGACCATTAGCAA |
| | | | AGCTAAGGGGCAGCCCAGGGAACCTCAGGTGTACACACTGCCTCCAAGTC |
| | | | GGGACGAGCTGACTAAAAACCAGGTCAGCCTGACCTGTCTGGTGAAGGGT |
| | | | TTTTATCCAAGCGATATCGCAGTGGAGTGGGAATCTAATGGCCAGCCCGAG |
| | | | AACAATTACAAGACTACCCCCCCTGTGCTGGACTCTGATGGTAGTTTCTTTC |
| | | | TGTATTCTAAACTGACCGTGGATAAGAGTAGGTGGCAGCAGGGCAACGTC |
| | | | TTCTCATGCTCCGTGATGCATGAAGCTCTGCACAATCATTACACCCAGAAAA |
| | | | GCCTGTCTCTGAGTCCTGGAAAGTGATAA |
| 213 | HuA5E3-IgG1 | IgG1 hc | MACPGFLWALVISTCLEFSMAQVQLQQSGPEVKKPGASVKISCKASGY |
| | (IQR) | | IFTSYYIQWVIQRPGQGLEWIGWIYPGNVNTKYNEKFKGKATLTADKSS |
| | | | STAYMELSSLTSEDTAVYFCARMDYEAHYWGQGTIVIVSSASTKGPSV |
| | | | FPLAPSSKSTSGGTAALGCLVKDYFPEPVTVSWNSGALTSGVHTFPAVL |
| | | | QSSGLYSLSSVVIVPSSSLGTQTYICNVNHKPSNTINDKINEPKSCDKTH |
| | | | TCPPCPAPELLGGPSVFLFPPKPKDTLMISRTPEVTCVVVDVSHEDPEVK |
| | | | FNWYVDGVEVHNAKTKPREEQYNSTYRVVSVLTVLHQDWLNGKEYK |
| | | | CINSNKALPAPIEKTISKAKGQPREPQVYTLPPSRDELTKNQVSLTCLVK |
| | | | GFYPSDIAVEWESNGQPENNYKTIPPVLDSDGSFFLYSKLTVDKSRWQ |
| | | | QGNVFSCSVMHEALHNHYTQKSLSLSPGK |
| 214 | HuA5E3-IgG1 | IgG1 hc | ATGGCATGCCCTGGCTTCCTGTGGGCACTTGTGATCTCCACCTGTCTTGAAT |
| | (IQR) | | TTTCCATGGCTCAGGTCCAGCTGCAGCAGAGCGGTCCCGAGGTCAAAAAAC |
| | | | CCGGCGCATCCGTGAAAATCAGTTGTAAAGCATCCGGTTATATCTTCACCTC |
| | | | CTACTATATCCAGTGGGTCATCCAGAGGCCTGGTCAGGGACTGGAATGGAT |
| | | | CGGATGGATCTACCCTGGGAACGTGAATACAAAGTATAACGAGAAGTTCA |
| | | | AAGGCAAGGCTACACTGACTGCAGACAAGTCCAGCTCTACTGCATACATGG |
| | | | AGCTGAGTTCACTGACTAGCGAAGACACCGCCGTGTATTTCTGCGCTAGAA |
| | | | TGGATTACGAAGCCCACTATTGGGGACAGGGGACCACAGTCACCGTGTCCT |
| | | | CCGCCAGCACAAAAGGTCCTTCCGTGTTCCCTCTGGCACCATCTAGTAAGTC |
| | | | TACAAGTGGCGGAACTGCCGCTCTGGGCTGTCTGGTGAAGGATTACTTCCC |
| | | | TGAGCCAGTCACCGTGTCCTGGAACAGCGGTGCACTGACTTCTGGCGTCCA |
| | | | TACCTTTCCAGCCGTGCTGCAGTCATCCGGACTGTACTCCCTGAGCTCTGTG |
| | | | GTCACTGTCCCCAGTTCATCCCTGGGGACCCAGACATATATCTGCAACGTGA |
| | | | ATCACAAACCTTCTAATACAAAGGTCGACAAGAAAGTGGAACCAAAATCCT |
| | | | GTGATAAGACTCATACCTGCCCACCTTGTCCAGCTCCTGAGCTGCTGGGAG |
| | | | GTCCAAGCGTGTTCCTGTTTCCACCCAAACCCAAGGACACCCTGATGATTAG |
| | | | CCGGACCCCTGAAGTCACATGCGTGGTCGTGGACGTGTCTCACGAGGATCC |
| | | | AGAAGTCAAGTTCAACTGGTACGTGGATGGCGTCGAGGTGCATAATGCCA |
| | | | AAACCAAGCCACGAGAGGAACAGTACAACAGTACATATCGTGTCGTGTCA |
| | | | GTCCTGACTGTGCTGCACCAGGACTGGCTGAACGGAAAGGAGTATAAATG |
| | | | CAAGGTGTCCAACAAGGCCCTGCCAGCCCCCATCGAGAAGACCATTAGCAA |
| | | | AGCTAAGGGGCAGCCCAGGGAACCTCAGGTGTACACACTGCCTCCAAGTC |
| | | | GGGACGAGCTGACTAAAAACCAGGTCAGCCTGACCTGTCTGGTGAAGGGT |
| | | | TTTTATCCAAGCGATATCGCAGTGGAGTGGGAATCTAATGGCCAGCCCGAG |
| | | | AACAATTACAAGACTACCCCCCCTGTGCTGGACTCTGATGGTAGTTTCTTTC |
| | | | TGTATTCTAAACTGACCGTGGATAAGAGTAGGTGGCAGCAGGGCAACGTC |
| | | | TTCTCATGCTCCGTGATGCATGAAGCTCTGCACAATCATTACACCCAGAAAA |
| | | | GCCTGTCTCTGAGTCCTGGAAAGTGATAA |
| 215 | HuA5E3-IgG1 | IgG1 hc | MACPGFLWALVISTCLEFSMAQVQLQQSGPEVKKPGASVKISCKASGY |
| | (KQR) | | IFTSYYIQWVKQRPGQGLEWIGWIYPGNVNTKYNEKFKGKATLTADKS |
| | | | SSTAYMELSSLTSEDTAVYFCARMDYEAHYWGQGTIVIVSSASTKGPS |
| | | | VFPLAPSSKSTSGGTAALGCLVKDYFPEPVTVSWNSGALTSGVHTFPAV |
| | | | LQSSGLYSLSSVVTVPSSSLGTQTYICNVNHKPSNTKVDKKVEPKSCDKT |
| | | | HTCPPCPAPELLGGPSVFLFPPKPKDTLMISRTPEVTCVVVDVSHEDPE |
| | | | VKFNWYVDGVEVHNAKTKPREEQYNSTYRVVSVLTVLHQDWLNGKE |
| | | | YKCKVSNKALPAPIEKTISKAKGQPREPQVYTLPPSRDELTKNQVSLTCL |
| | | | VKGFYPSDIAVEWESNGQPENNYKTIPPVLDSDGSFFLYSKLTVDKSR |
| | | | WQQGNVFSCSVMHEALHNHYTQKSLSLSPGK |
| 216 | HuA5E3-IgG1 | IgG1 hc | ATGGCATGCCCTGGCTTCCTGTGGGCACTTGTGATCTCCACCTGTCTTGAAT |
| | (KQR) | | TTTCCATGGCTCAGGTCCAGCTGCAGCAGAGCGGTCCCGAGGTCAAAAAAC |
| | | | CCGGCGCATCCGTGAAAATCAGTTGTAAAGCATCCGGTTATATCTTCACCTC |
| | | | CTACTATATCCAGTGGGTCAAGCAGAGGCCTGGTCAGGGACTGGAATGGA |
| | | | TCGGATGGATCTACCCTGGGAACGTGAATACAAAGTATAACGAGAAGTTCA |
| | | | AAGGCAAGGCTACACTGACTGCAGACAAGTCCAGCTCTACTGCATACATGG |
| | | | AGCTGAGTTCACTGACTAGCGAAGACACCGCCGTGTATTTCTGCGCTAGAA |
| | | | TGGATTACGAAGCCCACTATTGGGGACAGGGGACCACAGTCACCGTGTCCT |
| | | | CCGCCAGCACAAAAGGTCCTTCCGTGTTCCCTCTGGCACCATCTAGTAAGTC |
| | | | TACAAGTGGCGGAACTGCCGCTCTGGGCTGTCTGGTGAAGGATTACTTCCC |
| | | | TGAGCCAGTCACCGTGTCCTGGAACAGCGGTGCACTGACTTCTGGCGTCCA |
| | | | TACCTTTCCAGCCGTGCTGCAGTCATCCGGACTGTACTCCCTGAGCTCTGTG |
| | | | GTCACTGTCCCCAGTTCATCCCTGGGGACCCAGACATATATCTGCAACGTGA |
| | | | ATCACAAACCTTCTAATACAAAGGTCGACAAGAAAGTGGAACCAAAATCCT |
| | | | GTGATAAGACTCATACCTGCCCACCTTGTCCAGCTCCTGAGCTGCTGGGAG |
| | | | GTCCAAGCGTGTTCCTGTTTCCACCCAAACCCAAGGACACCCTGATGATTAG |
| | | | CCGGACCCCTGAAGTCACATGCGTGGTCGTGGACGTGTCTCACGAGGATCC |
| | | | AGAAGTCAAGTTCAACTGGTACGTGGATGGCGTCGAGGTGCATAATGCCA |
| | | | AAACCAAGCCACGAGAGGAACAGTACAACAGTACATATCGTGTCGTGTCA |
| | | | GTCCTGACTGTGCTGCACCAGGACTGGCTGAACGGAAAGGAGTATAAATG |
| | | | CAAGGTGTCCAACAAGGCCCTGCCAGCCCCCATCGAGAAGACCATTAGCAA |
| | | | AGCTAAGGGGCAGCCCAGGGAACCTCAGGTGTACACACTGCCTCCAAGTC |
| | | | GGGACGAGCTGACTAAAAACCAGGTCAGCCTGACCTGTCTGGTGAAGGGT |
| | | | TTTTATCCAAGCGATATCGCAGTGGAGTGGGAATCTAATGGCCAGCCCGAG |
| | | | AACAATTACAAGACTACCCCCCCTGTGCTGGACTCTGATGGTAGTTTCTTTC |
| | | | TGTATTCTAAACTGACCGTGGATAAGAGTAGGTGGCAGCAGGGCAACGTC |
| | | | TTCTCATGCTCCGTGATGCATGAAGCTCTGCACAATCATTACACCCAGAAAA |
| | | | GCCTGTCTCTGAGTCCTGGAAAGTGATAA |
| 217 | HuA5E3-IgG1 | VH | QVQLQQSGPEVKKPGASVKISCKASGYIFTSYYIQWVKHRPGQGLEWI |
| | (KHR) | | GWIYPGNVNTKYNEKFKGKATLTADKSSSTAYMELSSLTSEDTAVYFCA |
| | | | RMDYEAHYWGQGTIVIVSS |
| 218 | HuA5E3-IgG1 | VH | QVQLQQSGPEVKKPGASVKISCKASGYIFTSYYIQWVIHAPGQGLEWIA |
| | (49A) | | WIYPGNVNTKYNEKFKGKATLTADKSSSTAYMELSSLTSEDTAVYFCAR |
| | | | MDYEAHYWGQGTTVTVSS |
| 219 | HuA5E3-IgG1 | VH | QVQLQQSGPEVVKPGASVKISCKASGYIFTSYYIQWVIHAPGQGLEWI |
| | (12V) | | GWIYPGNVNTKYNEKFKGKATLTADKSSSTAYMELSSLTSEDTAVYFCA |
| | | | RMDYEAHYWGQGTIVIVSS |
| 220 | HuA5E3-IgG1 | VH | QVQLQQSGPEVKKPGASVKISCKASGYIFTSYYIQWVIHAPGQGLEWV |
| | (VAY) | | AYIYPGNVNTKYNEKFKGKATLTADKSSSTAYMELSSLTSEDTAVYFCAR |
| | | | MDYEAHYWGQGTTVTVSS |
| 221 | HuA5E3-IgG1 | VH | QVQLQQSGPEVKKPGASVKISCKASGYIFTSYYIQWVIHAPGQGLEWV |
| | (VAW) | | AWIYPGNVNTKYNEKFKGKATLTADKSSSTAYMELSSLTSEDTAVYFCA |
| | | | RMDYEAHYWGQGTIVIVSS |
| 222 | 5R48D-AFF | PRIMER | GTGACAATCACTTGTgacGCTTCCCAGGAAATT |
| 223 | 3R48D-AFF | PRIMER | AATTTCCTGGGAAGCgtcACAAGTGATTGTCAC |
| 224 | 5S50D-AFF | PRIMER | ATCACTTGTCGCGCTgacCAGGAAATTAGCGGA |
| 225 | 3S50D-AFF | PRIMER | TCCGCTAATTTCCTGgtcAGCGCGACAAGTGAT |
| 226 | 5Q51D-AFF | PRIMER | ACTTGTCGCGCTTCCgacGAAATTAGCGGATAC |
| 227 | 3Q51D-AFF | PRIMER | GTATCCGCTAATTTCgtcGGAAGCGCGACAAGT |
| 228 | 5E52D-AFF | PRIMER | TGTCGCGCTTCCCAGgacATTAGCGGATACCTG |
| 229 | 3E52D-AFF | PRIMER | CAGGTATCCGCTAATgtcCTGGGAAGCGCGACA |
| 230 | 5S54D-AFF | PRIMER | GCTTCCCAGGAAATTgacGGATACCTGACTTGG |
| 231 | 3S54D-AFF | PRIMER | CCAAGTCAGGTATCCgtcAATTTCCTGGGAAGC |
| 232 | 5A74D-AFF | PRIMER | AAGCGACTGATCTACgacGCATCTACCCTGGAC |
| 233 | 3A74D-AFF | PRIMER | GTCCAGGGTAGATGCgtcGTAGATCAGTCGCTT |
| 234 | 5S76D-AFF | PRIMER | CTGATCTACGCTGCAgacACCCTGGACAGTGGA |
| 235 | 3S76D-AFF | PRIMER | TCCACTGTCCAGGGTgtcTGCAGCGTAGATCAG |
| 236 | 5T77D-AFF | PRIMER | ATCTACGCTGCATCTgacCTGGACAGTGGAGTG |
| 237 | 3T77D-AFF | PRIMER | CACTCCACTGTCCAGgtcAGATGCAGCGTAGAT |
| 238 | 5L78D-AFF | PRIMER | TACGCTGCATCTACCgacGACAGTGGAGTGCCT |
| 239 | 3L78D-AFF | PRIMER | AGGCACTCCACTGTCgtcGGTAGATGCAGCGTA |
| 240 | 5S80D-AFF | PRIMER | GCATCTACCCTGGACgacGGAGTGCCTAAGAGG |
| 241 | 3S80D-AFF | PRIMER | CCTCTTAGGCACTCCgtcGTCCAGGGTAGATGC |
| 242 | 5G81D-AFF | PRIMER | TCTACCCTGGACAGTgacGTGCCTAAGAGGTTC |
| 243 | 3G81D-AFF | PRIMER | GAACCICTTAGGCACgtcACTGICCAGGGTAGA |
| 244 | 5A116D-AFF | PRIMER | AACTGCCTGCAGTACgacAATTATCCTAGAACA |
| 245 | 3A116D-AFF | PRIMER | TGTTCTAGGATAATTgtcGTACTGCAGGCAGTT |
| 246 | 5N117D-AFF | PRIMER | TGCCTGCAGTACGCCgacTATCCTAGAACATTT |
| 247 | 3N117D-AFF | PRIMER | AAATGTTCTAGGATAgtcGGCGTACTGCAGGCA |
| 248 | 5T183D-AFF | PRIMER | TCCGGTTATATCTTCgacTCCTACTATATCCAG |
| 249 | 3T183D-AFF | PRIMER | CTGGATATAGTAGGAgtcGAAGATATAACCGGA |
| 250 | 5S184D-AFF | PRIMER | GGTTATATCTTCACCgacTACTATATCCAGTGG |
| 251 | 3S184D-AFF | PRIMER | CCACTGGATATAGTAgtcGGTGAAGATATAACC |
| 252 | 5G207D-AFF | PRIMER | GGATGGATCTACCCTgacAACGTGAATACAAAG |
| 253 | 3G207D-AFF | PRIMER | CTTTGTATTCACGTTgtcAGGGTAGATCCATCC |
| 254 | 5N208D-AFF | PRIMER | TGGATCTACCCTGGGgacGTGAATACAAAGTAT |
| 255 | 3N208D-AFF | PRIMER | ATACTTTGTATTCACgtcCCCAGGGTAGATCCA |
| 256 | 5V209D-AFF | PRIMER | ATCTACCCTGGGAACgacAATACAAAGTATAAC |
| 257 | 3V209D-AFF | PRIMER | GTTATACTTTGTATTgtcGTTCCCAGGGTAGAT |
| 258 | 5N210D-AFF | PRIMER | TACCCTGGGAACGTGgacACAAAGTATAACGAG |
| 259 | 3N210D-AFF | PRIMER | CTCGTTATACTTTGTgtcCACGTTCCCAGGGTA |
| 260 | 5T211D-AFF | PRIMER | CCTGGGAACGTGAATgacAAGTATAACGAGAAG |
| 261 | 3T211D-AFF | PRIMER | CTTCTCGTTATACTTgtcATTCACGTTCCCAGG |
| 262 | 5K212D-AFF | PRIMER | GGGAACGTGAATACAgacTATAACGAGAAGTTC |
| 263 | 3K212D-AFF | PRIMER | GAACTTCTCGTTATAgtcTGTATTCACGTTCCC |
| 264 | 5E255D-AFF | PRIMER | GCTAGAATGGATTACgacGCCCACTATTGGGGA |
| 265 | 3E255D-AFF | PRIMER | TCCCCAATAGTGGGCgtcGTAATCCATTCTAGC |
| 266 | 5R48Y-AFF | PRIMER | GTGACAATCACTIGTtacGCTICCCAGGAAATT |
| 267 | 3R48Y-AFF | PRIMER | AATTTCCTGGGAAGCgtaACAAGTGATTGTCAC |
| 268 | 5S50Y-AFF | PRIMER | ATCACTIGTCGCGCTtacCAGGAAATTAGCGGA |
| 269 | 3S50Y-AFF | PRIMER | TCCGCTAATTTCCTGgtaAGCGCGACAAGTGAT |
| 270 | 5Q51Y-AFF | PRIMER | ACTTGTCGCGCTTCCtacGAAATTAGCGGATAC |
| 271 | 3Q51Y-AFF | PRIMER | GTATCCGCTAATTTCgtaGGAAGCGCGACAAGT |
| 272 | 5E52Y-AFF | PRIMER | TGTCGCGCTTCCCAGtacATTAGCGGATACCTG |
| 273 | 3E52Y-AFF | PRIMER | CAGGTATCCGCTAATgtaCTGGGAAGCGCGACA |
| 274 | 5S54Y-AFF | PRIMER | GCTICCCAGGAAATTtacGGATACCTGACTIGG |
| 275 | 3S54Y-AFF | PRIMER | CCAAGTCAGGTATCCgtaAATTTCCTGGGAAGC |
| 276 | 5A74Y-AFF | PRIMER | AAGCGACTGATCTACtacGCATCTACCCTGGAC |
| 277 | 3A74Y-AFF | PRIMER | GTCCAGGGTAGATGCgtaGTAGATCAGTCGCTT |
| 278 | 5S76Y-AFF | PRIMER | CTGATCTACGCTGCAtacACCCTGGACAGTGGA |
| 279 | 3S76Y-AFF | PRIMER | TCCACTGTCCAGGGTgtaTGCAGCGTAGATCAG |
| 280 | 5T77Y-AFF | PRIMER | ATCTACGCTGCATCTtacCIGGACAGIGGAGTG |
| 281 | 3T77Y-AFF | PRIMER | CACTCCACTGTCCAGgtaAGATGCAGCGTAGAT |
| 282 | 5L78Y-AFF | PRIMER | TACGCTGCATCTACCtacGACAGIGGAGTGCCT |
| 283 | 3L78Y-AFF | PRIMER | AGGCACTCCACTGTCgtaGGTAGATGCAGCGTA |
| 284 | 5S80Y-AFF | PRIMER | GCATCTACCCTGGACtacGGAGTGCCTAAGAGG |
| 285 | 3S80Y-AFF | PRIMER | CCTCTTAGGCACTCCgtaGTCCAGGGTAGATGC |
| 286 | 5G81Y-AFF | PRIMER | TCTACCCIGGACAGTtacGTGCCTAAGAGGITC |
| 287 | 3G81Y-AFF | PRIMER | GAACCTCTTAGGCACgtaACTGTCCAGGGTAGA |
| 288 | 5A116Y-AFF | PRIMER | AACTGCCTGCAGTACtacAATTATCCTAGAACA |
| 289 | 3A116Y-AFF | PRIMER | TGTTCTAGGATAATTgtaGTACTGCAGGCAGTT |
| 290 | 5N117Y-AFF | PRIMER | TGCCTGCAGTACGCCtacTATCCTAGAACATTT |
| 291 | 3N117Y-AFF | PRIMER | AAATGTTCTAGGATAgtaGGCGTACTGCAGGCA |
| 292 | 5T183Y-AFF | PRIMER | TCCGGTTATATCTTCtacTCCTACTATATCCAG |
| 293 | 3T183Y-AFF | PRIMER | CTGGATATAGTAGGAgtaGAAGATATAACCGGA |
| 294 | 5S184Y-AFF | PRIMER | GGTTATATCTTCACCtacTACTATATCCAGTGG |
| 295 | 3S184Y-AFF | PRIMER | CCACTGGATATAGTAgtaGGTGAAGATATAACC |
| 296 | 5G207Y-AFF | PRIMER | GGATGGATCTACCCItacAACGTGAATACAAAG |
| 297 | 3G207Y-AFF | PRIMER | CTTTGTATTCACGTTgtaAGGGTAGATCCATCC |
| 298 | 5N208Y-AFF | PRIMER | TGGATCTACCCTGGGtacGTGAATACAAAGTAT |
| 299 | 3N208Y-AFF | PRIMER | ATACTTTGTATTCACgtaCCCAGGGTAGATCCA |
| 300 | 5V209Y-AFF | PRIMER | ATCTACCCTGGGAACtacAATACAAAGTATAAC |
| 301 | 3V209Y-AFF | PRIMER | GTTATACTTTGTATTgtaGTTCCCAGGGTAGAT |
| 302 | 5N210Y-AFF | PRIMER | TACCCTGGGAACGTGtacACAAAGTATAACGAG |
| 303 | 3N210Y-AFF | PRIMER | CTCGTTATACTTTGTgtaCACGTTCCCAGGGTA |
| 304 | 5T211Y-AFF | PRIMER | CCIGGGAACGTGAATtacAAGTATAACGAGAAG |
| 305 | 3T211Y-AFF | PRIMER | CTTCTCGTTATACTTgtaATTCACGTTCCCAGG |
| 306 | 5K212Y-AFF | PRIMER | GGGAACGTGAATACAtacTATAACGAGAAGTTC |
| 307 | 3K212Y-AFF | PRIMER | GAACTTCTCGTTATAgtaTGTATTCACGTTCCC |
| 308 | 5E255Y-AFF | PRIMER | GCTAGAATGGATTACtacGCCCACTATTGGGGA |
| 309 | 3E255Y-AFF | PRIMER | TCCCCAATAGTGGGCgtaGTAATCCATTCTAGC |

The breadth and scope of the present disclosure should not be limited by any of the above-described exemplary embodiments, but should be defined only in accordance with the following claims and their equivalents.

What is claimed is:

1. An isolated binding molecule or antigen-binding fragment thereof comprising a humanized antibody heavy chain variable domain (VH) and a humanized antibody light chain variable domain (VL),
(a) wherein the VH is less than 100% identical to SEQ ID NO: 16 and comprises the amino acid structure HFW1-HCDR1-HFW2-HCDR2-HFW3-HCDR3-HFW4, wherein HFW1 is SEQ ID NO: 22, or SEQ ID NO: 22 with one, two, three, four, or five single amino acid substitutions; HCDR1 is SEQ ID NO: 17, or SEQ ID NO: 17 with one, two, or three single amino acid substitutions; HFW2 is SEQ ID NO: 26, or SEQ ID NO: 26 with one, two, three, four, or five single amino acid substitutions; HCDR2 is SEQ ID NO: 18, or SEQ ID NO: 18 with one, two, or three, single amino acid substitutions; HFW3 is SEQ ID NO: 45, or SEQ ID NO: 45 with one, two, three, four, or five single amino acid substitutions; HCDR3 is SEQ ID NO: 19, or SEQ ID NO: 19 with one, two, or three single amino acid substitutions; and HFW4 is SEQ ID NO: 48, or SEQ ID NO: 48 with one, two, or three single amino acid substitutions;
(b) wherein the VL is less than 100% identical to SEQ ID NO: 11 and comprises the amino acid structure LFW1-LCDR1-LFW2-LCDR2-LFW3-LCDR3-LFW4, wherein LFW1 is SEQ ID NO: 50, or SEQ ID NO: 50 with one, two, three, four, or five single amino acid substitutions; LCDR1 is SEQ ID NO: 12, or SEQ ID NO: 12 with one, two, or three single amino acid substitutions; LFW2 is SEQ ID NO: 52, or SEQ ID NO: 52 with one, two, three, four, or five single amino acid substitutions; LCDR2 is SEQ ID NO: 13, or SEQ ID NO: 13 with one single amino acid substitution; LFW3 is SEQ ID NO: 55, or SEQ ID NO: 55 with one, two, three, four, or five single amino acid substitutions; LCDR3 is SEQ ID NO: 14, or SEQ ID NO: 14 with one, two, or three single amino acid substitutions; and LFW4 is SEQ ID NO: 58, or SEQ ID NO: 58 with one, two, or three single amino acid substitutions; and
(c) wherein the binding molecule or fragment thereof can bind to a cyclic peptide comprising the amino acid sequence SNK, wherein the K (Lysine) is solvent-accessible.

2. The binding molecule or fragment thereof of claim 1, which is an isolated antibody or antigen-binding fragment thereof.

3. The antibody or fragment thereof of claim 2, wherein HFW1 is SEQ ID NO: 22, SEQ ID NO: 23, or SEQ ID NO: 24.

4. The antibody or fragment thereof of claim 2, wherein HCDR1 is SEQ ID NO: 17, SEQ ID NO: 21, SEQ ID NO: 190 or SEQ ID NO: 192.

5. The antibody or fragment thereof of claim 2, wherein HFW2 is SEQ ID NO: 26, SEQ ID NO: 27, SEQ ID NO: 29, SEQ ID NO: 31, SEQ ID NO: 33, SEQ ID NO: 35, SEQ ID NO: 37, SEQ ID NO: 38, SEQ ID NO: 39, SEQ ID NO: 40, SEQ ID NO: 41, SEQ ID NO: 42, SEQ ID NO: 43, or SEQ ID NO: 44.

6. The antibody or fragment thereof of claim 2, wherein HCDR2 is SEQ ID NO: 18, SEQ ID NO: 194, SEQ ID NO: 196, SEQ ID NO: 198, SEQ ID NO: 200, or SEQ ID NO: 202.

7. The antibody or fragment thereof of claim 2, wherein HFW3 is SEQ ID NO: 45, SEQ ID NO: 46, or SEQ ID NO: 47.

8. The antibody or fragment thereof of claim 2, wherein HCDR3 is SEQ ID NO: 19, SEQ ID NO: 98, SEQ ID NO: 100, SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 118, SEQ ID NO: 120, SEQ ID NO: 122, or SEQ ID NO: 206.

9. The antibody or fragment thereof of claim 2, wherein HFW4 is SEQ ID NO: 48 or SEQ ID NO: 49.

10. The antibody or fragment thereof of claim 2, wherein LFW1 is SEQ ID NO: 50 or SEQ ID NO: 51.

11. The antibody or fragment thereof of claim 2, wherein LCDR1 is SEQ ID NO: 12, SEQ ID NO: 168, SEQ ID NO: 170, or SEQ ID NO: 172.

12. The antibody or fragment thereof of claim 2, wherein LFW2 is SEQ ID NO: 52, SEQ ID NO: 53, or SEQ ID NO: 54.

13. The antibody or fragment thereof of claim 2, wherein LCDR2 is SEQ ID NO: 13, SEQ ID NO: 174, SEQ ID NO: 176, SEQ ID NO: 178, SEQ ID NO: 180, SEQ ID NO: 182, or SEQ ID NO: 184.

14. The antibody or fragment thereof of claim 2, wherein LFW3 is SEQ ID NO: 55, SEQ ID NO: 56, or SEQ ID NO: 57.

15. The antibody or fragment thereof of claim 2, wherein LCDR3 is SEQ ID NO: 14, SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 186, or SEQ ID NO: 188.

16. The antibody or fragment thereof of claim 2, wherein LFW4 is SEQ ID NO: 58 or SEQ ID NO: 59.

17. The antibody or fragment thereof of any one of claims 2 to 16, wherein the VH comprises the amino acid sequence SEQ ID NO: 63, SEQ ID NO: 67, SEQ ID NO: 71, SEQ ID NO: 124, SEQ ID NO: 126, SEQ ID NO: 128, SEQ ID NO: 130, SEQ ID NO: 132, SEQ ID NO: 144, SEQ ID NO: 146, SEQ ID NO: 148, SEQ ID NO: 150, SEQ ID NO: 152, SEQ ID NO: 154, SEQ ID NO: 156, SEQ ID NO: 158, SEQ ID NO: 160, SEQ ID NO: 162, SEQ ID NO: 217, SEQ ID NO: 218, SEQ ID NO: 219, SEQ ID NO: 220, or SEQ ID NO: 221.

18. The antibody or fragment thereof of any one of claims 2 to 16, wherein the VL comprises the amino acid sequence SEQ ID NO: 61, SEQ ID NO: 65, or SEQ ID NO: 69.

19. The antibody or fragment thereof of any one of claims 2 to 16, wherein the VH and VL comprise, respectively, the amino acid sequences SEQ ID NO: 63 and SEQ ID NO: 61, SEQ ID NO: 67 and SEQ ID NO: 65, SEQ ID NO: 71 and SEQ ID NO: 69, SEQ ID NO: 124 and SEQ ID NO: 65, SEQ ID NO: 126 and SEQ ID NO: 65, SEQ ID NO: 128 and SEQ ID NO: 65, SEQ ID NO: 130 and SEQ ID NO: 65, SEQ ID NO: 132 and SEQ ID NO: 65, SEQ ID NO: 144 and SEQ ID NO: 65, SEQ ID NO: 146 and SEQ ID NO: 65, SEQ ID NO: 148 and SEQ ID NO: 65, SEQ ID NO: 150 and SEQ ID NO: 65, SEQ ID NO: 152 and SEQ ID NO: 65, SEQ ID NO: 154 and SEQ ID NO: 65, SEQ ID NO: 156 and SEQ ID NO: 65, SEQ ID NO: 158 and SEQ ID NO: 65, SEQ ID NO: 160 and SEQ ID NO: 65, SEQ ID NO: 162 and SEQ ID NO: 65, SEQ ID NO: 217 and SEQ ID NO: 65, SEQ ID NO: 218 and SEQ ID NO: 65, SEQ ID NO: 219 and SEQ ID NO: 65, SEQ ID NO: 220 and SEQ ID NO: 65, or SEQ ID NO: 221 and SEQ ID NO: 65.

20. The antibody or fragment thereof of any one of claims 2 to 19 further comprising a light chain constant region or fragment thereof fused to the C-terminus of the VL.

21. The antibody or fragment thereof of claim 20, wherein the light chain constant region is a human kappa constant region.

22. The antibody or fragment thereof of any one of claims 2 to 21, further comprising a heavy chain constant region or fragment thereof fused to the C-terminus of the VH.

23. The antibody or fragment thereof of claim 22, wherein the heavy chain constant region is a human IgG constant region or a human IgA constant region.

24. The antibody or fragment thereof of claim 23, wherein the heavy chain constant region is a human IgG1 constant region, a human IgG2 constant region, or a human IgG4 constant region.

25. The antibody or fragment thereof of any one of claims 2 to 21, wherein the antigen-binding fragment is an Fv fragment, an Fab fragment, an F(ab′)2 fragment, an Fab′ fragment, a dsFv fragment, an scFv fragment, or an sc(Fv)2 fragment, or any combination thereof.

26. The antibody or fragment thereof of any one of claims 19 to 25, which exhibits enhanced expression in transiently transfected CHO cells as compared to a corresponding antibody or fragment thereof comprising the VH amino acid sequence SEQ ID NO: 67 and the VL amino acid sequence SEQ ID NO: 65.

27. The antibody or fragment thereof of claim 26, wherein the VL comprises the amino acid sequence SEQ ID NO: 65 and the VH comprises the amino acid sequence SEQ ID NO: 124, SEQ ID NO: 126, SEQ ID NO: 128, SEQ ID NO: 130, or SEQ ID NO: 132.

28. The antibody or fragment thereof of claim 26, wherein the VH and VL comprise, respectively, the amino acid sequences SEQ ID NO: 124 and SEQ ID NO: 65, SEQ ID NO: 126 and SEQ ID NO: 65, SEQ ID NO: 128 and SEQ ID NO: 65, SEQ ID NO: 130 and SEQ ID NO: 65, or SEQ ID NO: 132 and SEQ ID NO: 65.

29. The antibody or fragment thereof of any one of claims 2 to 28, wherein the cyclic peptide consists of 3, 4, 5, 6, 7, 8 or 9 amino acids.

30. The antibody or fragment thereof of claim 29, wherein the cyclic peptide comprises the amino acid sequence SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9.

31. The antibody or fragment thereof of any one of claims 2 to 28, wherein the dissociation constant (K_{D}) of the antibody, or antigen-binding fragment thereof, is less than about 1×10⁻⁸ M.

32. The antibody or fragment thereof of any one of claims 2 to 28, which can bind to an oligomeric form of Aβ at a greater affinity than to a non-oligomeric form of Aβ.

33. A composition comprising the binding molecule or fragment thereof of claim 1 or the antibody or fragment thereof of any one of claims 2 to 32, and a pharmaceutically acceptable carrier.

34. An isolated polynucleotide comprising a nucleic acid that encodes the binding molecule or fragment thereof of claim 1 or the antibody or fragment thereof of any one of claims 2 to 32, or a polypeptide subunit thereof.

35. A vector comprising the polynucleotide of claim 34.

36. A cell comprising the polynucleotide of claim 34 or the vector of claim 35.

37. The cell of claim 36, wherein the cell is a bacterial cell.

38. The cell of claim 36, wherein the cell is a eukaryotic cell.

39. The cell of claim 38, wherein the cell is a mammalian cell.

40. The cell of claim 39, wherein the cell is COS-1, COS-7, HEK293, BHK21, CHO, BSC-1, Hep G2, SP2/0, HeLa, myeloma or lymphoma cells.

41. A method of preventing or treating Alzheimer's disease comprising administering to a subject an effective amount of the antibody or fragment thereof of any one of claims 2 to 32.

42. The method of claim 41, wherein the antibody or fragment thereof is administered intravenously, subcutaneously, intramuscularly, intrathecally or transdermally.

43. The method of claim 41 or claim 42, further comprising the step of administering to the subject a second agent.
